(19) United States
(12) Patent Application Publication
(10) Pub. No.: US 2024/0262899 A1
(43) Pub. Date: Aug. 8, 2024

(54) ANTI-CITH3 ANTIBODIES AND USES THEREOF

(71) Applicants: University of Virginia Patent Foundation, Charlottesville, VA (US); HTIC, Inc., Charlottesville, VA (US); The Regents of the University of Michigan, Ann Arbor, MI (US)

(72) Inventors: Jianjie Ma, Keswick, VA (US); Chuanxi Cai, Glen Allen, VA (US); Yongqing Li, Ann Arbor, MI (US); Tao Tan, Columbus, OH (US); Yue Liu, San Mateo, CA (US)

(21) Appl. No.: 18/410,405

(22) Filed: Jan. 11, 2024

Related U.S. Application Data
(60) Provisional application No. 63/438,778, filed on Jan. 12, 2023.

(51) Int. Cl. C07K 16/18; A61K 38/17; A61K 39/395; A61P 9/10; A61P 17/02; A61P 37/06

(52) U.S. Cl. CPC C07K 16/18; A61K 38/1709; A61K 39/3955; A61P 9/10; A61P 17/02; A61P 37/06; C07K 2317/24; C07K 2317/31; C07K 2317/622

(57) ABSTRACT

This disclosure relates to anti-CitH3 (citrullinated histone H3) antibodies, antigen-binding fragments, and the uses thereof.

### CROSS-REFERENCE TO RELATED APPLICATIONS

This application claims priority to U.S. Provisional Application 63/438,778 filed on Jan. 12, 2023, which is incorporated herein by reference in its entirety.

### INCORPORATION OF ELECTRONICALLY FILED MATERIAL

The Instant Application contains a Sequence Listing which has been submitted electronically in XML format and is hereby incorporated by reference in its entirety. Said XML copy, created on Dec. 27, 2023, is named “HNC0002US” and is 21,126 bytes in size.

### TECHNICAL FIELD

This disclosure relates to anti-CitH3 (citrullinated histone H3) antibodies and uses thereof.

### BACKGROUND

Neutrophil extracellular traps (NETs) are networks of extracellular fibers, primarily composed of DNA from neutrophils, which bind pathogens. Neutrophils are the immune system's first line of defense against infection and have conventionally been thought to kill invading pathogens.

NETs can have a deleterious effect on the host, because the extracellular exposure of histone complexes can play a role during the development of autoimmune diseases like systemic lupus erythematosus. NETs can also play a role in inflammatory diseases, as NETs can be identified in preeclampsia, a pregnancy-related inflammatory disorder in which neutrophils are known to be activated.

Excessive NETs have been detected in sepsis and are associated with significant organ injury. Histone citrullination/deimination induced by peptidyl arginine deiminases (PADs) is an important post-translational modification that facilitates chromatin decondensation during NET formation. Moreover, citrullinated histones are found in the extracellular space of neutrophils along with DNA as components of NETs.

Citrullinated histone H3 (CitH3) has been recently shown to be highly involved in the process of NETosis. In clinical studies, the levels of CitH3 were found to be significantly correlated with respiratory sequential organ failure assessment score. Importantly, the released CitH3 triggers formation of more NETs through a positive feedback mechanism in neutrophils. In addition, CitH3 also actives pyroptosis of macrophages to cause further tissue injury and exacerbates immune dysfunction. This phenomenon is called a “vicious circle”, generating more circulating CitH3 and inducing “cytokine storm”.

The elevated levels of CitH3 have also been detected in patients with various cancers, which cause changes in the cancer micro-environment and therefore impact the progression of tumor and the efficacy of cancer treatment. Moreover, CitH3/NET-induced immune dysfunction can also impact the tissue repair and regeneration process, e.g. chronic wounds and multi-organ failures.

Recent clinical and commercial success of antibodies has created great interest in antibody-based therapeutics. There is a need to develop antibodies against CitH3 for treating immune dysfunctions caused by NET activation and release.

### SUMMARY

This disclosure relates to anti-CitH3 antibodies, antigen-binding fragment thereof, and the uses thereof.

In one aspect, the disclosure provides an antibody or antigen-binding fragment thereof that binds to CitH3 (citrullinated histone H3) comprising: a heavy chain variable region (VH) comprising complementarity determining regions (CDRs) 1, 2, and 3, wherein the VH CDR1 region comprises an amino acid sequence that is at least 80% identical to a selected VH CDR1 amino acid sequence, the VH CDR2 region comprises an amino acid sequence that is at least 80% identical to a selected VH CDR2 amino acid sequence, and the VH CDR3 region comprises an amino acid sequence that is at least 80% identical to a selected VH CDR3 amino acid sequence; and a light chain variable region (VL) comprising CDRs 1, 2, and 3, wherein the VL CDR1 region comprises an amino acid sequence that is at least 80% identical to a selected VL CDR1 amino acid sequence, the VL CDR2 region comprises an amino acid sequence that is at least 80% identical to a selected VL CDR2 amino acid sequence, and the VL CDR3 region comprises an amino acid sequence that is at least 80% identical to a selected VL CDR3 amino acid sequence. In some embodiments, the selected VH CDRs 1, 2, and 3 amino acid sequences and the selected VL CDRs, 1, 2, and 3 amino acid sequences are one of the following: (1) the selected VH CDRs 1, 2, 3 amino acid sequences are set forth in SEQ ID NOs: 1, 2, 3, respectively, and the selected VL CDRs 1, 2, 3 amino acid sequences are set forth in SEQ ID NOs: 4, 5, 6, respectively; and (2) the selected VH CDRs 1, 2, 3 amino acid sequences are set forth in SEQ ID NOs: 16, 17, 18, respectively, and the selected VL CDRs 1, 2, 3 amino acid sequences are set forth in SEQ ID NOs: 19, 20, 21, respectively.

In some embodiments, the VH comprises CDRs 1, 2, 3 with the amino acid sequences set forth in SEQ ID NOs: 1, 2, and 3 respectively, and the VL comprises CDRs 1, 2, 3 with the amino acid sequences set forth in SEQ ID NOs: 4, 5, and 6, respectively.

In some embodiments, the VH comprises CDRs 1, 2, 3 with the amino acid sequences set forth in SEQ ID NOs: 16, 17, and 18 respectively, and the VL comprises CDRs 1, 2, 3 with the amino acid sequences set forth in SEQ ID NOs: 19, 20, and 21, respectively.

In some embodiments, the antibody or antigen-binding fragment specifically binds to human CitH3.

In some embodiments, the antibody or antigen-binding fragment is a humanized antibody or antigen-binding fragment thereof.

In some embodiments, the antibody or antigen-binding fragment is a single-chain variable fragment (scFv) or a multi-specific antibody (e.g., a bispecific antibody).

In one aspect, the disclosure provides a nucleic acid comprising a polynucleotide encoding a polypeptide comprising: (1) an immunoglobulin heavy chain or a fragment thereof comprising a heavy chain variable region (VH) comprising complementarity determining regions (CDRs) 1, 2, and 3 comprising the amino acid sequences set forth in SEQ ID NOs: 1, 2, and 3, respectively, and wherein the VH, when paired with a light chain variable region (VL) comprising the amino acid sequence set forth in SEQ ID NO: 11, 12, 13, or 15, binds to CitH3; or (2) an immunoglobulin light chain or a fragment thereof comprising a VL comprising CDRs 1, 2, and 3 comprising the amino acid sequences set forth in SEQ ID NOs: 4, 5, and 6, respectively, and wherein the VL, when paired with a VH comprising the amino acid sequence set forth in SEQ ID NO: 7, 8, 9, 10, or 14, binds to CitH3.

In some embodiments, the nucleic acid comprises a polynucleotide encoding a polypeptide comprising an immunoglobulin heavy chain or a fragment thereof comprising a VH comprising CDRs 1, 2, and 3 comprising the amino acid sequences set forth in SEQ ID NOs: 1, 2, and 3, respectively.

In some embodiments, the nucleic acid comprises a polynucleotide encoding a polypeptide comprising an immunoglobulin light chain or a fragment thereof comprising a VL comprising CDRs 1, 2, and 3 comprising the amino acid sequences set forth in SEQ ID NOs: 4, 5, and 6, respectively.

In some embodiments, the VH when paired with a VL specifically binds to human CitH3, or the VL when paired with a VH specifically binds to human CitH3.

In some embodiments, the immunoglobulin heavy chain or the fragment thereof is a humanized immunoglobulin heavy chain or a fragment thereof, and the immunoglobulin light chain or the fragment thereof is a humanized immunoglobulin light chain or a fragment thereof.

In some embodiments, the nucleic acid encodes a single-chain variable fragment (scFv) or a multi-specific antibody (e.g., a bispecific antibody).

In some embodiments, the nucleic acid is cDNA.

In one aspect, the disclosure provides a vector comprising one or more of the nucleic acids as described herein. In one aspect, the disclosure provides a vector comprising two of the nucleic acids as described herein. In some embodiments, the vector encodes the VL region and the VH region that together bind to CitH3.

In one aspect, the disclosure provides a pair of vectors, wherein each vector comprises one of the nucleic acids as described herein. In some embodiments, the pair of vectors encodes the VL region and the VH region that together bind to CitH3.

In one aspect, the disclosure provides a cell comprising the vector as described herein, or the pair of vectors as described herein.

In some embodiments, the cell is a CHO cell.

In one aspect, the disclosure provides a cell comprising one or more of the nucleic acids as described herein.

In one aspect, the disclosure provides a cell comprising two of the nucleic acids as described herein.

In some embodiments, the two nucleic acids together encode the VL region and the VH region that together bind to CitH3.

In one aspect, the disclosure provides a method of producing an antibody or an antigen-binding fragment thereof, the method comprising
(a) culturing the cell as described herein under conditions sufficient for the cell to produce the antibody or the antigen-binding fragment; and
(b) collecting the antibody or the antigen-binding fragment produced by the cell.

In one aspect, the disclosure provides an antibody or antigen-binding fragment thereof that binds to CitH3 comprising a heavy chain variable region (VH) comprising an amino acid sequence that is at least 90%, 95%, or 100% identical to SEQ ID NO: 7, 8, 9, 10, or 14, and a light chain variable region (VL) comprising an amino acid sequence that is at least 90%, 95%, or 100% identical to SEQ ID NO: 11, 12, 13, or 15.

In some embodiments, the VH comprises the sequence of SEQ ID NO: 7 and the VL comprises the sequence of SEQ ID NO: 11.

In some embodiments, the VH comprises the sequence of SEQ ID NO: 7 and the VL comprises the sequence of SEQ ID NO: 12.

In some embodiments, the VH comprises the sequence of SEQ ID NO: 7 and the VL comprises the sequence of SEQ ID NO: 13.

In some embodiments, the VH comprises the sequence of SEQ ID NO: 8 and the VL comprises the sequence of SEQ ID NO: 11.

In some embodiments, the VH comprises the sequence of SEQ ID NO: 8 and the VL comprises the sequence of SEQ ID NO: 12.

In some embodiments, the VH comprises the sequence of SEQ ID NO: 8 and the VL comprises the sequence of SEQ ID NO: 13.

In some embodiments, the VH comprises the sequence of SEQ ID NO: 9 and the VL comprises the sequence of SEQ ID NO: 11.

In some embodiments, the VH comprises the sequence of SEQ ID NO: 9 and the VL comprises the sequence of SEQ ID NO: 12.

In some embodiments, the VH comprises the sequence of SEQ ID NO: 9 and the VL comprises the sequence of SEQ ID NO: 13.

In some embodiments, the VH comprises the sequence of SEQ ID NO: 10 and the VL comprises the sequence of SEQ ID NO: 11.

In some embodiments, the VH comprises the sequence of SEQ ID NO: 10 and the VL comprises the sequence of SEQ ID NO: 12.

In some embodiments, the VH comprises the sequence of SEQ ID NO: 10 and the VL comprises the sequence of SEQ ID NO: 13.

In some embodiments, the VH comprises the sequence of SEQ ID NO: 14 and the VL comprises the sequence of SEQ ID NO: 15.

In some embodiments, the antibody or antigen-binding fragment specifically binds to human CitH3.

In some embodiments, the antibody or antigen-binding fragment is a humanized antibody or antigen-binding fragment thereof.

In some embodiments, the antibody or antigen-binding fragment is a single-chain variable fragment (scFv) or a multi-specific antibody (e.g., a bispecific antibody).

In one aspect, the disclosure provides an antibody or antigen-binding fragment thereof that binds CitH3 comprising a heavy chain variable region (VH) comprising VH CDR1, VH CDR2, and VH CDR3 that are identical to VH CDR1, VH CDR2, and VH CDR3 of SEQ ID NO: 7, 8, 9, 10, or 14; and a light chain variable region (VL) comprising VL CDR1, VL CDR2, and VL CDR3 that are identical to VL CDR1, VL CDR2, and VL CDR3 of SEQ ID NO: 11, 12, 13, or 15.

In one aspect, the disclosure provides an antibody or antigen-binding fragment thereof that binds to CitH3 comprising a heavy chain variable region (VH) comprising VH CDR1, VH CDR2, and VH CDR3 that are identical to VH CDR1, VH CDR2, and VH CDR3 of a selected antibody or antigen-binding fragment thereof; and a light chain variable region (VL) comprising VL CDR1, VL CDR2, and VL CDR3 that are identical to VL CDR1, VL CDR2, and VL CDR3 of the selected antibody or antigen-binding fragment thereof,

In some embodiments, the selected antibody or antigen-binding fragment thereof is the antibody or antigen-binding fragment thereof as described herein.

In one aspect, the disclosure provides an antibody or antigen-binding fragment thereof that cross-competes with the antibody or antigen-binding fragment thereof as described herein.

In one aspect, the disclosure provides a method of treating a subject having cancer, the method comprising administering a therapeutically effective amount of a composition comprising the antibody or antigen-binding fragment thereof of as described herein to the subject.

In some embodiments, the subject has a solid tumor or hematological malignancy.

In some embodiments, the cancer is associated with CitH3-induced NETosis.

In one aspect, the disclosure provides a method of treating a subject having an immune disorder (e.g., autoimmune disease), the method comprising administering a therapeutically effective amount of a composition comprising the antibody or antigen-binding fragment thereof as described herein to the subject. In some embodiments, the immune disorder is associated with NETosis.

In one aspect, the disclosure provides a method of treating a subject having an infectious disease, the method comprising administering a therapeutically effective amount of a composition comprising the antibody or antigen-binding fragment thereof as described herein to the subject.

In some embodiments, the infectious disease is caused by bacteria and/or viruses, e.g., sepsis.

In one aspect, the infectious disease is influenza.

In one aspect, the disclosure provides a method of inhibiting CitH3-NETosis in a subject, the method comprising administering a therapeutically effective amount of a composition comprising the antibody or antigen-binding fragment thereof as described herein to the subject.

In some embodiments, the CitH3-NETosis is caused by an infectious disease, sepsis, cancer, chronic wounds or an autoimmune disorder.

In one aspect, the disclosure provides a method for treating a subject having an organ injury, such as a cardiac, liver, or brain infarction, the method comprising administering a therapeutically effective amount of a composition comprising the antibody or antigen-binding fragment thereof as described herein to the subject.

In one aspect, the disclosure provides a method for treating a subject having skin ulcers, e.g. diabetic foot ulcer, tissue injury or chronic wound impairment, the method comprising administering a therapeutically effective amount of a composition comprising the antibody or antigen-binding fragment thereof as described herein to the subject.

In one aspect, the disclosure provides a pharmaceutical composition comprising the antibody or antigen-binding fragment thereof as described herein, and a pharmaceutically acceptable carrier.

In one aspect, the disclosure provides an antibody-drug conjugate comprising the antibody or antigen-binding fragment thereof as described herein covalently bound to a therapeutic agent. In some embodiments, the therapeutic agent is a cytotoxic or cytostatic agent.

In one aspect, the disclosure provides a pharmaceutical composition comprising the antibody drug conjugate as described herein, and a pharmaceutically acceptable carrier.

As used herein, the term “antibody” refers to any antigen-binding molecule that contains at least one (e.g., one, two, three, four, five, or six) complementary determining region (CDR) (e.g., any of the three CDRs from an immunoglobulin light chain or any of the three CDRs from an immunoglobulin heavy chain) and is capable of specifically binding to an epitope. Non-limiting examples of antibodies include: monoclonal antibodies, polyclonal antibodies, multi-specific antibodies (e.g., bi-specific antibodies), single-chain antibodies, chimeric antibodies, human antibodies, and humanized antibodies. In some embodiments, an antibody can contain an Fc region of a human antibody. The term antibody also includes derivatives, e.g., bi-specific antibodies, single-chain antibodies, diabodies, linear antibodies, and multi-specific antibodies formed from antibody fragments.

As used herein, the term “antigen-binding fragment” refers to a portion of a full-length antibody, wherein the portion of the antibody is capable of specifically binding to an antigen. In some embodiments, the antigen-binding fragment contains at least one variable domain (e.g., a variable domain of a heavy chain or a variable domain of light chain). Non-limiting examples of antibody fragments include, e.g., Fab, Fab′, F(ab′)₂, and Fv fragments.

As used herein, the term “human antibody” refers to an antibody that is encoded by an endogenous nucleic acid (e.g., rearranged human immunoglobulin heavy or light chain locus) present in a human. In some embodiments, a human antibody is collected from a human or produced in a human cell culture (e.g., human hybridoma cells). In some embodiments, a human antibody is produced in a non-human cell (e.g., a mouse or hamster cell line). In some embodiments, a human antibody is produced in a bacterial or yeast cell. In some embodiments, a human antibody is produced in a transgenic non-human animal (e.g., a bovine) containing an unrearranged or rearranged human immunoglobulin locus (e.g., heavy or light chain human immunoglobulin locus).

As used herein, the term “chimeric antibody” refers to an antibody that contains a sequence present in at least two different antibodies (e.g., antibodies from two different mammalian species such as a human and a mouse antibody). A non-limiting example of a chimeric antibody is an antibody containing the variable domain sequences (e.g., all or part of a light chain and/or heavy chain variable domain sequence) of a non-human (e.g., mouse) antibody and the constant domains of a human antibody. Additional examples of chimeric antibodies are described herein and are known in the art.

As used herein, the term “humanized antibody” refers to a non-human antibody which contains minimal sequence derived from a non-human (e.g., mouse) immunoglobulin and contains sequences derived from a human immunoglobulin. In non-limiting examples, humanized antibodies are human antibodies (recipient antibody) in which hypervariable (e.g., CDR) region residues of the recipient antibody are replaced by hypervariable (e.g., CDR) region residues from a non-human antibody (e.g., a donor antibody), e.g., a mouse, rat, or rabbit antibody, having the desired specificity, affinity, and capacity. In some embodiments, the Fv framework residues of the human immunoglobulin are replaced by corresponding non-human (e.g., mouse) immunoglobulin residues. In some embodiments, humanized antibodies may contain residues which are not found in the recipient antibody or in the donor antibody. These modifications can be made to further refine antibody performance. In some embodiments, the humanized antibody contains substantially all of at least one, and typically two, variable domains, in which all or substantially all of the hypervariable loops (CDRs) correspond to those of a non-human (e.g., mouse) immunoglobulin and all or substantially all of the framework regions are those of a human immunoglobulin. The humanized antibody can also contain at least a portion of an immunoglobulin constant region (Fc), typically, that of a human immunoglobulin. Humanized antibodies can be produced using molecular biology methods known in the art. Non-limiting examples of methods for generating humanized antibodies are described herein.

As used herein, the terms “subject” and “patient” are used interchangeably throughout the specification and describe an animal, human or non-human, to whom treatment according to the methods of the present invention is provided. Veterinary and non-veterinary applications are contemplated by the present invention. Human patients can be adult humans or juvenile humans (e.g., humans below the age of 18 years old). In addition to humans, patients include but are not limited to mice, rats, hamsters, guinea-pigs, rabbits, ferrets, cats, dogs, and primates. Included are, for example, non-human primates (e.g., monkey, chimpanzee, gorilla, and the like), rodents (e.g., rats, mice, gerbils, hamsters, ferrets, rabbits), lagomorphs, swine (e.g., pig, miniature pig), equine, canine, feline, bovine, and other domestic, farm, and zoo animals.

As used herein, when referring to an antibody, the phrases “specifically binding” and “specifically binds” mean that the antibody interacts with its target molecule (e.g., CitH3) preferably to other molecules, because the interaction is dependent upon the presence of a particular structure (i.e., the antigenic determinant or epitope) on the target molecule; in other words, the reagent is recognizing and binding to molecules that include a specific structure rather than to all molecules in general. An antibody that specifically binds to the target molecule may be referred to as a target-specific antibody. For example, an antibody that specifically binds to a CitH3 molecule may be referred to as a CitH3-specific antibody or an anti-CitH3 antibody.

As used herein, the terms “polypeptide,” “peptide,” and “protein” are used interchangeably to refer to polymers of amino acids of any length of at least two amino acids.

As used herein, the terms “polynucleotide,” “nucleic acid molecule,” and “nucleic acid sequence” are used interchangeably herein to refer to polymers of nucleotides of any length of at least two nucleotides, and include, without limitation, DNA, RNA, DNA/RNA hybrids, and modifications thereof.

Unless otherwise defined, all technical and scientific terms used herein have the same meaning as commonly understood by one of ordinary skill in the art to which this invention belongs. Methods and materials are described herein for use in the present invention; other, suitable methods and materials known in the art can also be used. The materials, methods, and examples are illustrative only and not intended to be limiting. All publications, patent applications, patents, sequences, database entries, and other references mentioned herein are incorporated by reference in their entirety. In case of conflict, the present specification, including definitions, will control.

Other features and advantages of the invention will be apparent from the following detailed description and figures, and from the claims.

### DESCRIPTION OF DRAWINGS

FIG. 1 shows initial ELISA screening results of humanized antibodies binding to CitH3.

FIGS. 2A-2E show ELISA binding results of humanized antibodies untreated or under heat treatment. The chimeric antibody VH/VL was used as a control.

FIG. 3 shows non-specific baculovirus binding results of humanized antibodies by ELISA. 7A4E11 and Rituxan were used as negative controls.

FIG. 4 shows verified ELISA binding results of humanized antibodies binding to CitH3. 7A4E11 was used as a positive control.

FIG. 5 shows a non-reduced SDS-PAGE gel of the chimeric antibody VH/VL (lane 1), and three humanized antibodies VH2/VL2 (lane 2), VH2/VL3 (lane 3), and VH3/VL3 (lane 4). MW is a protein marker.

FIG. 6A shows the reducing-CE-SDS result of standard IgG.

FIG. 6B shows the reducing-CE-SDS result of humanized antibody VH3/VL3.

FIG. 6C shows the non-reducing-CE-SDS result of standard IgG.

FIG. 6D shows the non-reducing-CE-SDS result of humanized antibody VH3/VL3.

FIGS. 7A-B show comparative analysis of Cayman CitH3-mAb and hCitH3-mAb binding affinity. Four distinct peptides—histone H3 (H3), citrullinated histone H3 (CitH3; 4 Cit), acetylated histone H3 (AceH3), and methylated histone H3 (MetH3) (New England Peptide™, Inc., Gardner, MA)—at a concentration of 0.5 microgram each, were run in duplicate through SDS-polyacrylamide gel electrophoresis and then transferred to a nitrocellulose membrane. This membrane was split into two sections for parallel probing with an equal quantity of Cayman CitH3-mAb (Cayman Chemicals, Ann Arbor, MI) or hCitH3-mAb (2 μg/ml) overnight. For convenience, hCitH3-mAb is the same as VH3/VL3. Post three extensive washes, the sections were incubated with HRP-conjugated secondary antibodies specific to mouse IgG and human IgG, respectively, for 2 hours (7A). For concurrent signal development, both membrane sections were exposed simultaneously for the same duration. Quantitative analysis of the signal intensity was performed using Image Studio Lite, and the results are displayed as mean±SEM for three independent experiments (n=3/group). **: P<0.01 (7B).

FIGS. 8A-B show a schematic of an assay (8A) and results (8B) showing that hCitH3-mAb improves survival mice subjected to LPS-induced septic shock. C57BL6/J mice were exposed to a lethal LPS dose (25 mg/kg, i.p) that caused complete mortality within 24 hours (with IgG as a control). Tail vein administration of hCitH3-mAb (20 mg/kg, tail vein) significantly improved survival.

FIGS. 9A-C show developed multiplex platform using “pre-equilibrium digital ELISA (PEd-ELISA)” technology (9A and 9B) and results (9C) showing early detection of CitH3 elevation following endotoxic shock.

FIGS. 10A-B show a schematic of assay (10A) and results (10B) indicating that hCitH3-mAb (H3L3) improves survival of mice subjected to Pseudomonas aeruginosa (PA)-induced sepsis. C57BL6/J mice (10-12 weeks, male) subjected to PA-induced septic shock (2.5×10⁶ CFU, intranasal). hCitH3-mAb (20 mg/kg) were delivered to mice via tail vein injection at 30 min post PA-administration. As control, mice received similar treatment of human IgG (purchased from Sigma). Mice receiving the human IgG all died within 3 days (n=6), whereas ˜80% of those receiving hCitH3-mAb survived (n=5).

FIGS. 11A-B show results for histological evaluation (11A) of PA-induced ALI, separate experiments were conducted with mice subjected to PA-septic shock and sacrificed at 24 hours post hCitH3-mAb administration (n=5 for hCitH3-mAb, n=5 for IgG). Score of acute lung injury (ALI) was evaluated using mouse lung sections by a board-certified pathologist who was blinded to the animal treatment (11B).

FIGS. 12A-B show a schematic (12A) and survival curve results (12B) for C57BL/6 mice (12 weeks of age) treated with or without hCitH3-mAb in a mouse model of the cecal ligation and puncture (CLP) induced lethal sepsis. C57BL/6 mice were administered a single dose of hCitH3-mAb (20 mg/kg of body weight) or human IgG (20 mg/kg of body weight) 0.5 hour before CLP. Survival rates were monitored for 10 days (n=7 per group). The results indicate that hCitH3-mAb significantly improved mouse survival compared to the IgG group.

FIGS. 13A-C show results of evaluating hCitH3-mAb's role in bacterial clearance during septic shock induced by Pseudomonas aeruginosa. Mice were intranasally inoculated with 2.5×10⁶ Pseudomonas aeruginosa cells and then administered a tail vein injection of hCitH3-mAb (20 mg/kg) or human IgG (20 mg/kg). Post-infection, lung and spleen tissues were harvested and homogenized for bacterial load assessment by culturing on LB agar plates (13B—lung, 13C—spleen). Image of agar plates (13A).

FIG. 14 shows synergy of hCitH3-mAb and rhMG53 in protection against CLP-sepsis in aged mice. Aging C57BL/6J mice (male, 24-26 moths) were obtained from National Institute of Aging. Mice were established under anesthesia with isoflurane according to protocols approved by IACUC at University of Virginia. Mouse sepsis were performed with Cecal Ligation and Puncture (CLP) model. Mice cecum were isolated out of the peritoneal cavity with sterile tweezers, ligated with 4-0 silk sutures, and punctured using a 20 G needle at the midpoint between the tail end and the ligation set. Mice were divided into four groups: CLP, CLP treated with rhMG53, CLP treated with hCitH3-mAb, and CLP treated with rhMG53+hCitH3-mAb. hCitH3-mAb were administrated via tail vein injection with a dose of 20 mg/kg and recombinant human MG53 (rhMG53) protein was administered via intraperitoneal injection with a dose of 5 mg/kg after CLP.

FIG. 15 shows that compared with sham control, CLP induced elevation of IL-6 in aged mice. Administration of rhMG53 (5 mg/kg, intraperitoneal injection) or hCitH3-mAb (20 mg/kg, tail vein injection) mitigated the elevation of IL-6. Co-administration of rhMG53 and hCitH3-mAb nearly completely abolished the elevation of IL-6.

FIGS. 16A and 16B show the changes in body weight (% of initial body weight) plotted animals infected with flu virus and treated with rhMG53 (16A) or hCitH3-mAb (16B) versus animals treated with saline as control group.

FIGS. 17A-17D show representative microscope images for lung tissue section with H&E staining for mouse infected with flu virus and treated with saline (17A), rhMG53 (17B), or hCitH3-mAb (17C). The quantitative analysis of cellulalization in the tissue section (17D) were conducted using the ImageJ software and decreased cell infiltration was observed in the lung tissue collected from animals at 9 days after flu virus infection and treated with rhMG53 (or hCitH3-mAb compared to animals treated with the saline group. (n=3 per group, p values calculated using one-way ANOVA with Tukey's multiple-comparison test).

FIGS. 18A-18B show results of ELISA quantification of IFNβ (18A) and IL-6 (18B) in the lung tissue collected from mice without flu virus infection, or mice with 9-day flu virus infection and treated with saline, rhMG53 or hhCitH3-mAb (n=3 per group, p values calculated using one-way ANOVA with Tukey's multiple-comparison test).

FIGS. 19A-19C present results of multiplex assay for mouse cytokines showing increased plasma level of inflammatory markers, including GM-CSF (19B), IFNγ (19C), and LIX (19A), in mice with flu virus infection compared to uninfected control animals, which were significantly decreased in animals treated with rhMG53 or hCitH3-mAb (n=3 per group, p values calculated using one-way ANOVA with Tukey's multiple-comparison test).

FIG. 20 shows western blots showed that treatment with rhMG53 or hCitH3-mAb suppressed the inflammation as indicated by the decreased level of NLRP3, MDA-5, IFITM3 following 9-day influenza virus.

FIG. 21 shows western blots showed that treatment with rhMG53 or hCitH3-mAb suppressed the apoptosis as indicated by the decreased level of cleaved caspase 2/3/11 and PARP following 9-day influenza virus infection.

FIG. 22 shows western blots showed that treatment with rhMG53 or hCitH3-mAb suppressed the expression of full-length Gasdermin D and decreased the level of cleaved gasdermin D following 9 days of influenza virus infection.

FIG. 23 shows western blot analysis showed that the expression of MPO, a neutrophil marker, was observed in ischemia-reperfusion induced liver injury with upregulation detected from 6 hours up to 72 hours after reperfusion.

FIG. 24 shows western blot analysis showing expression of CitH3 increased in ischemia-reperfusion induced liver injury in mice at 24 hours after reperfusion and continued to increase up to 72 hours.

FIGS. 25A and 25B show that hCitH3-mAb treatment eliminated CitH3 level in serum by western blot analysis showed. (25A) Mice that underwent liver ischemia and reperfusion were injected with human IgG antibody or hCitH3-mAb via i.v. immediately after reperfusion. CitH3 levels were determined in serum samples collected before surgery and on days 1, 3, and 7 after surgery. (25B) shows the Ponceau S staining for the loading control.

FIG. 26 shows results of neutralization assay of CitH3 with hCitH3-mAb and restoration of cardiac function following liver ischemia and reperfusion.

FIGS. 27A and 27B shows images of whole-brain tissue from mice treated with CitH3-mAb vs. control. (27A) shows coronal section of harvested normal vs infarcted brain tissue samples from treated and control mice 24 h after reperfusion. (27B) The extent of ischemic infarction was traced and integrated volume was calculated and is shown in the bar graph.

FIG. 28 shows images of wound area of hCit-mAb treated wounds and untreated wounds in 14 week-old B6.BKS(D)-Lepr^{db}/J (db/db) mice. 14 week-old B6.BKS(D)-Lepr^{db}/J (db/db) mice were purchased from the Jackson Laboratory. After anesthesia using 1.5% isoflurane, the dorsal hair of mice was removed using a depilatory cream (Nair), and a 5 mm round full-thickness skin excision wound was made at midline of the back 2-cm caudal ward from the skull of each mouse. hCitH3-mAb (20 mg/kg) and human IgG (Sigma-Aldrich, I4506; 20 mg/kg) was subcutaneously injected from post-injury day 0 and day 3. The wounds were covered with a hydrocolloid dressing (Tegaderm; 3M Health Care) to maintain a moist environment and changed daily. Wound area was measured at post-injury day 2, 4, 7, 10, and 12. The data revealed benefits of hCitH3-mAb to improve healing of dermal wounds in the db/db mice (n=3 each).

FIGS. 29A-29C shows cotreatment of hCitH3-mAb and hydrogel formulation of recombinant human mitsugumin 53 (rhMG53) protein have synergy to control inflammation and improve healing of diabetic ulcer. rhMG53 is a TRIM (tripartite motif-containing) family protein.

### DETAILED DESCRIPTION

The present disclosure provides examples of antibodies, antigen-binding fragments thereof, that bind to CitH3 (citrullinated histone H3). In some embodiments, the antibodies or antigen-binding fragments thereof described herein can be used to treat immuno-dysfunction caused by multi-organ injuries and/or tumor growth that are associated with NETosis. In some embodiments, such conditions include infectious disease and sepsis caused by bacteria and/or viruses. In other embodiments, the conditions include cancer progression and/or autoimmune disorders, as well as chronic wounds and organ failure.

Histones bind and package nuclear DNA into nucleosomes, which can be released into the blood stream upon cell activation or damage. As such, several clinical studies indicate that circulating cell free DNA (cfDNA) and nucleosomes serve as potential blood markers in a variety of malignancies, but investigations of the diagnostic or prognostic relevance of circulating histones are fewer and focused mainly on sepsis and trauma patients. Once released extracellularly, histones can mediate injurious effects on the host, suggesting their potential as both prognostic markers and therapeutic targets.

Posttranslational histone modifications can have profound effects on their structure and function and have been linked to various diseases. Citrullinated histone H3 (CitH3) is the product of the posttranslational conversion of peptidylarginine to citrulline on the N-terminal of histone H3. The subsequent decrease in positive charge of the histone residue results in a weaker binding to the negatively charged DNA, leading to chromatin decondensation. Histone citrullination is catalyzed by the enzymes peptidylarginine deiminase 4 (PAD4) and PAD2, primarily located in the cytoplasm of immune cells and the only PAD isozymes able to translocate to the nucleus upon cell activation. This crucial role of CitH3 in immune cell chromatin decondensation has rendered CitH3 a central marker of the recently described neutrophil release of decondensed and web-like nuclear chromatin, termed neutrophil extracellular traps (NETs). CitH3 can be detected in the nucleus of neutrophils upon stimulation but also released into the bloodstream upon NETosis. Importantly, the released CitH3 can induce formation of more NETs through a positive feedback mechanism. Furthermore, PAD2 and PAD4 have been shown to be overexpressed in various types of tumors, suggesting that CitH3 could be released into the bloodstream upon cancer cell death. Tumor cells may thus be an additional and unrecognized source of CitH3 in cancer.

This disclosure provides several anti-CitH3 antibodies and humanized anti-CitH3 antibodies that can effectively inhibit NET activation and release and can be used to treat various diseases.

### Histone Citrullination

Citrullination is the Ca²⁺-driven enzymatic conversion of arginine residues to citrulline, catalyzed by the PAD family. Citrullination or deimination indicates modification of the primary ketimine group (═NH) to a ketone group (═O), yielding ammonia as a side-product. Thus, the strongly alkaline and positively charged arginine side chains are hydrolyzed to form a neutral urea. Charge transfer affects protein-protein interactions, hydrogen bond formation and protein structure, which may cause denaturation in some cases. Various proteins, such as cytoplasmic, nuclear, membrane and mitochondrial proteins, can be citrullinated. Citrullinated proteins can be detected using antibody-based detection systems, e.g., mass spectrometry.

Citrullinated histones account for approximately 10% of all histone molecules in HL-60 granulocytes, highlighting the significance of PTM in many nucleus-related processes. Various tumors are associated with the hyperexpression of PAD and increased citrullination. The challenge is identifying specific citrullinated proteins involved in various autoimmune diseases. Additionally, protein identification is the basis for further study of the mechanism of citrullination mechanisms. Specifically, histone citrullination affects tumorigenesis in various ways, such as regulating gene transcription, cell differentiation and apoptosis. The discovery of histone citrullination, a reaction in which histones are substrates for PAD, may be a breakthrough in tumor research.

Regarding tumor immunity, new epitopes induced by PTMs may provide new targets for tumor-specific immunotherapy. Nutrient deficiency, hypoxia, redox stress, DNA damage and other tumor microenvironmental conditions can increase the activity of PAD and expression of citrullinated protein. Recently, the average CitH3 concentration in late malignant tumor patient serum increased three-fold compared with that in healthy people, indicating that citrullinated proteins may be cancer biomarkers. In one group of cancer patients, the mean serum level of CitH3 in invasive tumors was higher than that in localized tumors, consistent with previous reports of PAD-mediated citrullination and metastasis. Notably, the level of CitH3 in the plasma of cancer patients was associated with higher levels of cell-free DNA and neutrophil activation. Remarkably, researchers found that a high plasma level of CitH3 (>29.8 ng/mL, more than 75%) is strongly associated with the risk of short-term death. The increased expression of CitH3 is considered a novel prognostic blood marker in patients with advanced cancer. The proportion of CitH3-positive neutrophils is increased in patients with more severe disease. The level of CitH3 in serum is closely related to neutrophil activation markers such as elastase, myeloperoxidase, IL-6 and IL-8. Therefore, CitH3 is considered a useful biomarker for evaluating the inflammatory response and prognosis of patients with advanced cancer.

That histone citrullination may also play a role in cancer therapy has also been reported. Venous thromboembolism (VTE) often occurs during cancer treatment and can be life-threatening. Studies have shown that CitH3 is independently associated with VTE in cancer patients and plays an important role in predicting VTE occurrence in cancer treatment. Additionally, inhibition of PAD2-mediated H3Cit26 decreases the expression of IL-6 in BMMSCs, mediating malignant plasma cell resistance to chemotherapeutic agents. One group found that an effective inhibitor of PAD can enhance antitumor activity by inhibiting CitH3 in an HCT-116 xenograft mouse model. Moreover, PAD2-H3Cit26 is considered a novel therapeutic target in castration-resistant prostate cancer. Furthermore, cross-talk between histones undergoing deacetylation and those undergoing citrullination is associated with cancer cell growth, indicating a combination of PAD and histone deacetylase inhibitors as a strategy for cancer treatment. In MCF-7 cells, C1-amidine regulates the expression of the tumor suppressor protein OKL38 by decreasing histone citrullination at the OKL38 promoter. Finally, ubiquitous compounds used in herbal medicine inhibit hematogenous metastasis of certain tumors by targeting CitH3 and NETs.

The present disclosure provides several anti-CitH3 antibodies, antigen-binding fragments thereof, and methods of using these anti-CitH3 antibodies and antigen-binding fragments to treat various diseases.

### Anti-CitH3 Antibodies and Antigen-Binding Fragments

The disclosure provides antibodies and antigen-binding fragments thereof that specifically bind to CitH3. The antibodies and antigen-binding fragments described herein are capable of specifically binding to CitH3, with a high purity and thermostability. The disclosure provides e.g., mouse anti-CitH3 antibodies Y1128, and chimeric antibodies, the humanized antibodies thereof.

The CDR sequences for Y1128, and Y1128 derived antibodies (e.g., humanized antibodies) include CDRs of the heavy chain variable domain, SEQ ID NOs: 1-3, and CDRs of the light chain variable domain, SEQ ID NOs: 4-6 as defined by Kabat numbering. The CDRs can also be defined by Chothia system. Under the Chothia numbering, the CDR sequences of the heavy chain variable domain are set forth in SEQ ID NOs: 16-18, and CDR sequences of the light chain variable domain are set forth in SEQ ID NOs: 19-21.

The amino acid sequence for heavy chain variable region and light variable region of humanized antibodies are also provided. As there are different ways to humanize the mouse antibody (e.g., sequence can be substituted by different amino acids), the heavy chain and the light chain of an antibody can have more than one version of humanized sequences. The amino acid sequences for the heavy chain variable region of humanized Y1128 antibody are set forth in SEQ ID NOs: 7-10. The amino acid sequences for the light chain variable region of humanized 9H3 antibody are set forth in SEQ ID NOs: 11-13. Any of these heavy chain variable region sequences (SEQ ID NO: 7-10) can be paired with any of these light chain variable region sequences (SEQ ID NO: 11-13).

As shown in Table 1, humanization percentage means the percentage identity of the heavy chain or light chain variable region sequence as compared to human antibody sequences in International Immunogenetics Information System (IMGT) database. The top hit means that the heavy chain or light chain variable region sequence is closer to a particular species than to other species. For example, top hit to human means that the sequence is closer to human than to other species. Top hit to mouse means that the sequence is closer to mouse than to other species, and these percentages identities are highest as compared to the sequences of other species. In some embodiments, humanization percentage is greater than 80%, 81%, 82%, 83%, 84%, 85%, 86%, 87%, 88%, 89%, 90%, 91%, 92%, 93%, 94%, or 95%. A detailed description regarding how to determine humanization percentage and how to determine top hits is known in the art. A high humanization percentage often has various advantages, e.g., more safe and more effective in humans, more likely to be tolerated by a human subject, and/or less likely to have side effects.

Furthermore, in some embodiments, the antibodies or antigen-binding fragments thereof described herein can also contain one, two, or three heavy chain variable region CDRs selected from the group of SEQ ID NOs: 1-3, and SEQ ID NOs: 16-18; and/or one, two, or three light chain variable region CDRs selected from the group of SEQ ID NOs: 4-6, and SEQ ID NOS 19-21.

TABLE 1

| Humanized | | | | |
|---|---|---|---|---|
| antibody | | | | SEQ |
| variable | | | | ID |
| domains | Top Hit | Description | AMINO ACID SEQUENCE | NO: |
| Humanized | Human | hu VHv1: | EVQLVQSGAEVKKPGATVKISCKASGFNIK | 7 |
| heavy chain | | humanization | DYYMHWMQQRPGKGLEWIGWIDPENGNTIY | |
| variable domain | | percentage | DPKFQGRVTITSDTSTDTAYLELSSLRSED | |
| (VH1) | | 83.5% | TAVYYCAPFGNYVWFAYWGQGTLVTVSS | |
| Humanized | Mouse | hu VHv2: | EVQVVQSGAEVKKPGATVKISCKASGFNIK | 8 |
| heavy chain | | humanization | DYYMHWMKQRPEQGLEWIGWIDPENGNTIY | |
| variable domain | | percentage | DPKFQGRVTITSDTSTDTAYLELSSLRSED | |
| (VH2) | | 79.4% | TAVYYCAPFGNYVWFAYWGQGTLVTVSS | |
| Humanized | Mouse | hu VHv3: | EVQVVQSGAEVKKPGATVKISCKASGFNIK | 9 |
| heavy chain | | humanization | DYYMHWMKQRPEQGLEWIGWIDPENGNTIY | |
| variable domain | | percentage | DPKFQGRATITSDTSTDTAYLELSSLRSED | |
| (VH3) | | 78.4% | TAVYYCAPFGNYVWFAYWGQGTLVTVSS | |
| Humanized | Mouse | hu VHv4: | EVQVQQSGAEVKKPGATVKLSCKASGFNIK | 10 |
| heavy chain | | humanization | DYYMHWMKQRPEQGLEWIGWIDPENGNTIY | |
| variable domain | | percentage | DPKFQGRATITSDTSTDTAYLELSSLRSED | |
| (VH4) | | 76.3% | TAVYYCAPFGNYVWFAYWGQGTLVTVSS | |
| Humanized | Human | hu VLv1: | DVVMTQSPLSLPVTLGQPASISCRSSQSIVH | 11 |
| light chain | | humanization | SNGITYLEWFQQRPGQSPRLLIYKVSNRFSG | |
| variable domain | | percentage | VPDRFSGSGSGTDFTLKISRVEAEDVGVYYC | |
| (VL1) | | 91% | FQGSHVPFTFGQGTKLEIKR | |
| Humanized | Mouse | hu VLv2: | DVVMTQSPLSLPVTLGQPASISCRSSQSIVH | 12 |
| light chain | | humanization | SNGITYLEWYLQRPGQSPKLLIYKVSNRFSG | |
| variable domain | | percentage | VPDRFSGSGSGTDFTLKISRVEAEDVGLYYC | |
| (VL2) | | 87% | FQGSHVPFTFGQGTKLEIKR | |
| Humanized | Mouse | hu VLv3: | DVLMTQSPLSLPVTLGQPASISCRSSQSIVH | 13 |
| light chain | | humanization | SNGITYLEWYLQRPGQSPKLLIYKVSNRFSG | |
| variable domain | | percentage | VPDRFSGSGSGTDFTLKISRVEAEDVGLYYC | |
| (VL3) | | 86% | FQGSHVPFTFGQGTKLEIKR | |

In some embodiments, the antibodies can have a heavy chain variable region (VH) comprising complementarity determining regions (CDRs) 1, 2, 3, wherein the CDR1 region comprises or consists of an amino acid sequence that is at least 80%, 85%, 90%, or 95% identical to a selected VH CDR1 amino acid sequence, the CDR2 region comprises or consists of an amino acid sequence that is at least 80%, 85%, 90%, or 95% identical to a selected VH CDR2 amino acid sequence, and the CDR3 region comprises or consists of an amino acid sequence that is at least 80%, 85%, 90%, or 95% identical to a selected VH CDR3 amino acid sequence, and a light chain variable region (VL) comprising CDRs 1, 2, 3, wherein the CDR1 region comprises or consists of an amino acid sequence that is at least 80%, 85%, 90%, or 95% identical to a selected VL CDR1 amino acid sequence, the CDR2 region comprises or consists of an amino acid sequence that is at least 80%, 85%, 90%, or 95% identical to a selected VL CDR2 amino acid sequence, and the CDR3 region comprises or consists of an amino acid sequence that is at least 80%, 85%, 90%, or 95% identical to a selected VL CDR3 amino acid sequence. The selected VH CDRs 1, 2, 3 amino acid sequences and the selected VL CDRs, 1, 2, 3 amino acid sequences are shown in Table 2 (Kabat CDR) and Table 3 (Chothia CDR).

TABLE 2
KABAT CDR

| VH | SEQ | VH | SEQ | VH | SEQ |
|---|---|---|---|---|---|
| CDR1 | ID: | CDR2 | ID: | CDR3 | ID: |
| DYYMH | 1 | WIDPENGNTIYDPKFQG | 2 | FGNYVWFAY | 3 |

| VL | SEQ | VL | SEQ | VL | SEQ |
|---|---|---|---|---|---|
| CDR1 | ID: | CDR2 | ID: | CDR3 | ID: |
| RSSQSIVHSNGITYLE | 4 | KVSNRFS | 5 | FQGSHVPFT | 6 |

TABLE 3
CHOTHIA CDR

| VH | SEQ | VH | SEQ | VH | SEQ | VL | SEQ | VL | SEQ | VL | SEQ |
|---|---|---|---|---|---|---|---|---|---|---|---|
| CDR1 | ID: | CDR2 | ID: | CDR3 | ID: | CDR1 | ID: | CDR2 | ID: | CDR3 | ID: |
| GFNIKDY | 16 | DPENGN | 17 | FGNYVWFAY | 18 | RSSQSIVHSNGITYLE | 19 | KVSNRFS | 20 | FQGSHVPFT | 21 |

In some embodiments, the antibody or an antigen-binding fragment described herein can contain a heavy chain variable domain containing one, two, or three of the CDRs of SEQ ID NO: 1 with zero, one or two amino acid insertions, deletions, or substitutions; SEQ ID NO: 2 with zero, one or two amino acid insertions, deletions, or substitutions; SEQ ID NO: 3 with zero, one or two amino acid insertions, deletions, or substitutions.

In some embodiments, the antibody or an antigen-binding fragment described herein can contain a heavy chain variable domain containing one, two, or three of the CDRs of SEQ ID NO: 16 with zero, one or two amino acid insertions, deletions, or substitutions; SEQ ID NO: 17 with zero, one or two amino acid insertions, deletions, or substitutions; SEQ ID NO: 18 with zero, one or two amino acid insertions, deletions, or substitutions.

In some embodiments, the antibody or an antigen-binding fragment described herein can contain a light chain variable domain containing one, two, or three of the CDRs of SEQ ID NO: 4 with zero, one or two amino acid insertions, deletions, or substitutions; SEQ ID NO: 5 with zero, one or two amino acid insertions, deletions, or substitutions; SEQ ID NO: 6 with zero, one or two amino acid insertions, deletions, or substitutions.

In some embodiments, the antibody or an antigen-binding fragment described herein can contain a light chain variable domain containing one, two, or three of the CDRs of SEQ ID NO: 19 with zero, one or two amino acid insertions, deletions, or substitutions; SEQ ID NO: 20 with zero, one or two amino acid insertions, deletions, or substitutions; SEQ ID NO: 21 with zero, one or two amino acid insertions, deletions, or substitutions.

The insertions, deletions, and substitutions can be within the CDR sequence, or at one or both terminal ends of the CDR sequence.

The disclosure also provides antibodies or antigen-binding fragments thereof that bind to CitH3. The antibodies or antigen-binding fragments thereof contain a heavy chain variable region (VH) comprising or consisting of an amino acid sequence that is at least 80%, 85%, 90%, or 95% identical to a selected VH sequence, and a light chain variable region (VL) comprising or consisting of an amino acid sequence that is at least 80%, 85%, 90%, or 95% identical to a selected VL sequence. In some embodiments, the selected VH sequence is SEQ ID NOs: 7, 8, 9, 10, or 14, and the selected VL sequence is SEQ ID NOs: 11, 12, 13, or 15.

To determine the percent identity of two amino acid sequences, or of two nucleic acid sequences, the sequences are aligned for optimal comparison purposes (e.g., gaps can be introduced in one or both of a first and a second amino acid or nucleic acid sequence for optimal alignment and non-homologous sequences can be disregarded for comparison purposes). The amino acid residues or nucleotides at corresponding amino acid positions or nucleotide positions are then compared. When a position in the first sequence is occupied by the same amino acid residue or nucleotide as the corresponding position in the second sequence, then the molecules are identical at that position. The percent identity between the two sequences is a function of the number of identical positions shared by the sequences, taking into account the number of gaps, and the length of each gap, which need to be introduced for optimal alignment of the two sequences. For purposes of the present disclosure, the comparison of sequences and determination of percent identity between two sequences can be accomplished using a Blossum 62 scoring matrix with a gap penalty of 12, a gap extend penalty of 4, and a frameshift gap penalty of 5.

The disclosure also provides nucleic acid comprising a polynucleotide encoding a polypeptide comprising an immunoglobulin heavy chain or an immunoglobulin heavy chain. The immunoglobulin heavy chain or immunoglobulin light chain comprises CDRs as shown in Table 2 or Table 3, or have sequences as shown in Table 1 or in SEQ ID NOs. 14, 15 and 22. When the polypeptides are paired with corresponding polypeptide (e.g., a corresponding heavy chain variable region or a corresponding light chain variable region), the paired polypeptides bind to CitH3 (e.g., human CitH3).

The anti-CitH3 antibodies and antigen-binding fragments can also be antibody variants (including derivatives and conjugates) of antibodies or antibody fragments and multi-specific (e.g., bi-specific) antibodies or antibody fragments. Additional antibodies provided herein are polyclonal, monoclonal, multi-specific (multimeric, e.g., bi-specific), human antibodies, chimeric antibodies (e.g., human-mouse chimera), single-chain antibodies, intracellularly-made antibodies (i.e., intrabodies), and antigen-binding fragments thereof. The antibodies or antigen-binding fragments thereof can be of any type (e.g., IgG, IgE, IgM, IgD, IgA, and IgY), class (e.g., IgG1, IgG2, lgG3, IgG4, IgA1, and IgA2), or subclass. In some embodiments, the antibody or antigen-binding fragment thereof is an IgG antibody or antigen-binding fragment thereof.

Fragments of antibodies are suitable for use in the methods provided so long as they retain the desired affinity and specificity of the full-length antibody. Thus, a fragment of an antibody that binds to CitH3 will retain an ability to bind to CitH3. An Fv fragment is an antibody fragment which contains a complete antigen recognition and binding site. This region consists of a dimer of one heavy and one light chain variable domain in tight association, which can be covalent in nature, for example in scFv. It is in this configuration that the three CDRs of each variable domain interact to define an antigen binding site on the surface of the VH-VL dimer. Collectively, the six CDRs or a subset thereof confer antigen binding specificity to the antibody. However, even a single variable domain (or half of an Fv comprising only three CDRs specific for an antigen) can have the ability to recognize and bind antigen, although usually at a lower affinity than the entire binding site.

Single-chain Fv or (scFv) antibody fragments comprise the VH and VL domains (or regions) of antibody, wherein these domains are present in a single polypeptide chain. Generally, the scFv polypeptide further comprises a polypeptide linker between the VH and VL domains, which enables the scFv to form the desired structure for antigen binding.

The Fab fragment contains a variable and constant domain of the light chain and a variable domain and the first constant domain (CH1) of the heavy chain. F(ab′)2 antibody fragments comprise a pair of Fab fragments which are generally covalently linked near their carboxy termini by hinge cysteines between them. Other chemical couplings of antibody fragments are also known in the art.

Diabodies are small antibody fragments with two antigen-binding sites, which fragments comprise a VH connected to a VL in the same polypeptide chain (VH and VL). By using a linker that is too short to allow pairing between the two domains on the same chain, the domains are forced to pair with the complementary domains of another chain and create two antigen-binding sites.

Linear antibodies comprise a pair of tandem Fd segments (VH-CH1-VH-CH1) which, together with complementary light chain polypeptides, form a pair of antigen binding regions. Linear antibodies can be bispecific or monospecific.

Antibodies and antibody fragments of the present disclosure can be modified in the Fc region to provide desired effector functions or serum half-life.

Multimerization of antibodies may be accomplished through natural aggregation of antibodies or through chemical or recombinant linking techniques known in the art. For example, some percentage of purified antibody preparations (e.g., purified IgG₁ molecules) spontaneously form protein aggregates containing antibody homodimers and other higher-order antibody multimers.

Alternatively, antibody homodimers may be formed through chemical linkage techniques known in the art. For example, heterobifunctional crosslinking agents including, but not limited to SMCC (succinimidyl 4-(maleimidomethyl)cyclohexane-1-carboxylate) and SATA (N-succinimidyl S-acethylthio-acetate) can be used to form antibody multimers. Antibody homodimers can be converted to Fab′₂ homodimers through digestion with pepsin. Another way to form antibody homodimers is through the use of the autophilic T15 peptide.

In some embodiments, the multi-specific antibody is a bi-specific antibody. Bi-specific antibodies can be made by engineering the interface between a pair of antibody molecules to maximize the percentage of heterodimers that are recovered from recombinant cell culture. For example, the interface can contain at least a part of the CH3 domain of an antibody constant domain. In this method, one or more small amino acid side chains from the interface of the first antibody molecule are replaced with larger side chains (e.g., tyrosine or tryptophan). Compensatory “cavities” of identical or similar size to the large side chain(s) are created on the interface of the second antibody molecule by replacing large amino acid side chains with smaller ones (e.g., alanine or threonine). This provides a mechanism for increasing the yield of the heterodimer over other unwanted end-products such as homodimers. Methods for preparing bi-specific antibodies are known in the art.

Bi-specific antibodies include cross-linked or “heteroconjugate” antibodies. For example, one of the antibodies in the heteroconjugate can be coupled to avidin and the other to biotin. Heteroconjugate antibodies can also be made using any convenient cross-linking methods. Suitable cross-linking agents and cross-linking techniques are well known in the art.

Methods for generating bi-specific antibodies from antibody fragments are also known in the art. For example, bi-specific antibodies can be prepared using chemical linkage, starting with a procedure where intact antibodies are proteolytically cleaved to generate F(ab′)₂ fragments. These fragments are reduced in the presence of the dithiol complexing agent sodium arsenite to stabilize vicinal dithiols and prevent intermolecular disulfide formation. The Fab′ fragments generated are then converted to thionitrobenzoate (TNB) derivatives. One of the Fab′ TNB derivatives is then reconverted to the Fab′ thiol by reduction with mercaptoethylamine and is mixed with an equimolar amount of another Fab′ TNB derivative to form the bi-specific antibody.

Any of the antibodies or antigen-binding fragments described herein may be conjugated to a stabilizing molecule (e.g., a molecule that increases the half-life of the antibody or antigen-binding fragment thereof in a subject or in solution). Non-limiting examples of stabilizing molecules include: a polymer (e.g., a polyethylene glycol) or a protein (e.g., serum albumin, such as human serum albumin). The conjugation of a stabilizing molecule can increase the half-life or extend the biological activity of an antibody or an antigen-binding fragment in vitro (e.g., in tissue culture or when stored as a pharmaceutical composition) or in vivo (e.g., in a human).

In some embodiments, the antibodies or antigen-binding fragments described herein can be conjugated to a therapeutic agent. The antibody-drug conjugate comprising the antibody or antigen-binding fragment thereof can covalently or non-covalently bind to a therapeutic agent. In some embodiments, the therapeutic agent is a cytotoxic or cytostatic agent (e.g., cytochalasin B, gramicidin D, ethidium bromide, emetine, mitomycin, etoposide, tenoposide, vincristine, vinblastine, colchicin, doxorubicin, daunorubicin, dihydroxy anthracin, maytansinoids such as DM-1 and DM-4, dione, mitoxantrone, mithramycin, actinomycin D, 1-dehydrotestosterone, glucocorticoids, procaine, tetracaine, lidocaine, propranolol, puromycin, epirubicin, and cyclophosphamide and analogs).

### Antibodies and Antigen Binding Fragments

The present disclosure provides anti-CitH3 antibodies and antigen-binding fragments thereof. In general, antibodies (also called immunoglobulins) are made up of two classes of polypeptide chains, light chains and heavy chains. A non-limiting antibody of the present disclosure can be an intact, four immunoglobulin chain antibody comprising two heavy chains and two light chains. The heavy chain of the antibody can be of any isotype including IgM, IgG, IgE, IgA, or IgD or sub-isotype including IgG1, IgG2, IgG2a, IgG2b, IgG3, IgG4, IgE1, IgE2, etc. The light chain can be a kappa light chain or a lambda light chain. An antibody can comprise two identical copies of a light chain and two identical copies of a heavy chain. The heavy chains, which each contain one variable domain (or variable region, V_{H}) and multiple constant domains (or constant regions), bind to one another via disulfide bonding within their constant domains to form the “stem” of the antibody. The light chains, which each contain one variable domain (or variable region, V_{L}) and one constant domain (or constant region), each bind to one heavy chain via disulfide binding. The variable region of each light chain is aligned with the variable region of the heavy chain to which it is bound. The variable regions of both the light chains and heavy chains contain three hypervariable regions sandwiched between more conserved framework regions (FR).

These hypervariable regions, known as the complementary determining regions (CDRs), form loops that comprise the principal antigen binding surface of the antibody. The four framework regions largely adopt a beta-sheet conformation and the CDRs form loops connecting, and in some cases forming part of, the beta-sheet structure. The CDRs in each chain are held in close proximity by the framework regions and, with the CDRs from the other chain, contribute to the formation of the antigen-binding region.

Methods for identifying the CDR regions of an antibody by analyzing the amino acid sequence of the antibody are well known, and a number of definitions of the CDRs are commonly used. The Kabat definition is based on sequence variability, and the Chothia definition is based on the location of the structural loop regions. Unless specifically indicated in the present disclosure, Kabat numbering is used in the present disclosure as a default.

The CDRs are important for recognizing an epitope of an antigen. As used herein, an “epitope” is the smallest portion of a target molecule capable of being specifically bound by the antigen binding domain of an antibody. The minimal size of an epitope may be about three, four, five, six, or seven amino acids, but these amino acids need not be in a consecutive linear sequence of the antigen's primary structure, as the epitope may depend on an antigen's three-dimensional configuration based on the antigen's secondary and tertiary structure.

In some embodiments, the antibody is an intact immunoglobulin molecule (e.g., IgG1, IgG2a, IgG2b, IgG3, IgM, IgD, IgE, IgA). The IgG subclasses (IgG1, IgG2, IgG3, and IgG4) are highly conserved, differing in their constant region, particularly in their hinges and upper CH2 domains. The sequences and differences of the IgG subclasses are known in the art.

The antibody can also be an immunoglobulin molecule that is derived from any species (e.g., human, rodent, mouse, camelid). Antibodies disclosed herein also include, but are not limited to, polyclonal, monoclonal, monospecific, polyspecific antibodies, and chimeric antibodies that include an immunoglobulin binding domain fused to another polypeptide. The term “antigen binding domain” or “antigen binding fragment” is a portion of an antibody that retains specific binding activity of the intact antibody, i.e., any portion of an antibody that is capable of specific binding to an epitope on the intact antibody's target molecule. It includes, e.g., Fab, Fab′, F(ab′)₂, and variants of these fragments. Thus, in some embodiments, an antibody or an antigen binding fragment thereof can be, e.g., a scFv, a Fv, a Fd, a dAb, a bispecific antibody, a bispecific scFv, a diabody, a linear antibody, a single-chain antibody molecule, a multi-specific antibody formed from antibody fragments, and any polypeptide that includes a binding domain which is, or is homologous to, an antibody binding domain. Non-limiting examples of antigen binding domains include, e.g., the heavy chain and/or light chain CDRs of an intact antibody, the heavy and/or light chain variable regions of an intact antibody, full length heavy or light chains of an intact antibody, or an individual CDR from either the heavy chain or the light chain of an intact antibody.

In some embodiments, the antigen binding fragment can form a part of a chimeric antigen receptor (CAR). In some embodiments, the chimeric antigen receptor are fusions of single-chain variable fragments (scFv) as described herein, fused to CD3-zeta transmembrane-and endodomain. In some embodiments, the chimeric antigen receptor also comprises intracellular signaling domains from various costimulatory protein receptors (e.g., CD28, 41BB, ICOS). In some embodiments, the chimeric antigen receptor comprises multiple signaling domains, e.g., CD3z-CD28-41BB or CD3z-CD28-CitH3, to increase potency. Thus, in one aspect, the disclosure further provides cells (e.g., T cells) that express the chimeric antigen receptors as described herein.

In some embodiments, the scFv has one heavy chain variable domain, and one light chain variable domain.

The present disclosure also provides an antibody or antigen-binding fragment thereof that cross-competes with any antibody or antigen-binding fragment as described herein. The cross-competing assay is known in the art. In one aspect, the present disclosure also provides an antibody or antigen-binding fragment thereof that binds to the same epitope or region as any antibody or antigen-binding fragment as described herein. The epitope binding assay is known in the art.

### Antibody Characteristics

In some embodiments, the antibodies or antigen-binding fragments thereof as described herein are CitH3 agonist. In some embodiments, the antibodies or antigen-binding fragments thereof are CitH3 antagonist.

In some implementations, the antibody (or antigen-binding fragments thereof) specifically binds to CitH3 (e.g., human CitH3, monkey CitH3, mouse CitH3, and/or chimeric CitH3) with a dissociation rate (koff) of less than 0.1 s⁻¹, less than 0.01 s⁻¹, less than 0.001 s⁻¹, less than 0.0001 s⁻¹, or less than 0.0001 s⁻¹. In some embodiments, the dissociation rate (koff) is greater than 0.01 s⁻¹, greater than 0.001 s⁻¹, greater than 0.0001 s⁻¹, greater than 0.0001 s⁻¹, or greater than 0.00001 s⁻¹.

In some embodiments, kinetic association rates (kon) is greater than 1×10²/Ms, greater than 1×10³/Ms, greater than 1×10⁴/Ms, greater than 1×10⁵/Ms, or greater than 1×10⁶/Ms. In some embodiments, kinetic association rates (kon) is less than 1×10⁵/Ms, less than 1×10⁶/Ms, or less than 1×10⁷/Ms.

Affinities can be deduced from the quotient of the kinetic rate constants (KD=koff/kon). In some embodiments, KD is less than 1×10⁻⁶ M, less than 1×10⁻⁷ M, less than 1×10⁻⁸ M, less than 1×10⁻⁹ M, or less than 1×10⁻¹⁰ M. In some embodiments, the KD is less than 30 nM, 20 nM, 15 nM, 10 nM, 9 nM, 8 nM, 7 nM, 6 nM, 5 nM, 4 nM, 3 nM, 2 nM, or 1 nM. In some embodiments, KD is greater than 1×10⁻⁷ M, greater than 1×10⁻⁸ M, greater than 1×10⁻⁹ M, greater than 1×10⁻¹⁰ M, greater than 1×10⁻¹¹ M, or greater than 1×10⁻¹² M.

General techniques for measuring the affinity of an antibody for an antigen include, e.g., ELISA, RIA, and surface plasmon resonance (SPR). In some embodiments, the antibody binds to human CitH3 (SEQ ID NO: 22), monkey CitH3, and/or mouse CitH3. In some embodiments, the antibody does not bind to human CitH3, monkey CitH3, and/or mouse CitH3.

In some embodiments, the antibodies or antigen binding fragments thereof as described herein are resistant to heat treatment after incubation at about 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, or 95° C. for at least 1, 2, 3, 4, 5, 6, 7, 8, 9, 10, 20, 30, 40, 50, or 60 minutes. In some embodiments, the heat resistance is determined by measuring the binding between the antibodies or antigen binding fragments and the antigen (e.g., CitH3). For example, loss of CitH3 binding as compared to the untreated samples indicates heat vulnerability of the antibodies or antigen binding fragments.

In some embodiments, the antibodies or antigen binding fragments thereof as described herein have a binding capacity to baculovirus that is comparable (e.g., less than about 150%, about 140%, about 130%, about 120%, about 110%, about 100%, about 90%, about 80%, about 70%, about 60%, or about 50%) as compared to a reference antibody (e.g., 7A4E11 or Rituxan).

In some embodiments, the antibodies or antigen binding fragments thereof as described herein (e.g., humanized anti-CitH3 antibodies) have a binding capacity to CitH3 that is at least 50%, at least 60%, at least 70%, at least 80%, at least 90%, at least 100%, at least 110%, at least 120%, at least 130%, at least 140%, at least 150%, at least 160%, at least 170%, at least 180%, at least 190%, or at least 200% as compared to that of the chimeric anti-CitH3 antibody described herein or a reference antibody (e.g., 7A4E11).

In some embodiments, the antibodies or antigen binding fragments thereof as described herein can be purified with a purity of at least 90%, 91%, 92%, 93%, 94%, 95%, 95.5%, 96%, 96.5%, 97%, 97.5%, 98%, 98.5%, 99%, 99.5%, 99.6%, 99.7%, 99.8%, or 99.9%.

In some embodiments, the thermostability of the antibodies or antigen binding fragments thereof is determined, e.g., by differential scanning fluorimetry (DSF). The antibodies or antigen binding fragments as described herein can have a Tm greater than 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, or 95° C. As IgG can be described as a multi-domain protein, the melting curve sometimes shows multiple transitions, with a first denaturation temperature, Tm1, a second denaturation temperature Tm2, and a third denaturation temperature Tm3. The presence of these three peaks often indicates the denaturation of the CH2 domain (Tm1), CH3 domain (Tm2) and Fab domains (Tm3), respectively. Thus, in some embodiments, the antibodies or antigen binding fragments as described herein has a Tm1 greater than 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, or 95° C. In some embodiments, the antibodies or antigen binding fragments as described herein has a Tm2 greater than 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, or 95° C. In some embodiments, Tm1 and Tm2 are less than 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, or 95° C.

In some embodiments, the thermal aggregation risk of the antibodies or antigen binding fragments thereof is determined, e.g., by static light scattering (SLS). Tagg is the temperature at which SLS starts to detect aggregation. In some embodiments, the Tagg 266 (for detection of smaller aggregation particles) is less than 60, 61, 62, 63, 64, 65, 66, 67, 68, 69, 70, 71, 72, 73, 74, 75, 76, 77, 78, 79, or 80° C. In some embodiments, the Tagg 473 (for detection of larger aggregation particles) is less than 60, 61, 62, 63, 64, 65, 66, 67, 68, 69, 70, 71, 72, 73, 74, 75, 76, 77, 78, 79, or 80° C. In some embodiments, the Tagg 266 and/or Tagg 473 are greater than 60, 61, 62, 63, 64, 65, 66, 67, 68, 69, 70, 71, 72, 73, 74, 75, 76, 77, 78, 79, or 80° C.

In some embodiments, the aggregation of the antibodies or antigen binding fragments is determined, e.g., by dynamic light scattering (DLS). In some embodiments, the polydispersity index (PDI) of the antibodies or antigen binding fragments thereof is no greater than 0.3, no greater than 0.29, no greater than 0.28, no greater than 0.27, no greater than 0.26, no greater than 0.25, no greater than 0.24, no greater than 0.23, no greater than 0.22, no greater than 0.21, no greater than 0.2, or no greater than 0.19. In some embodiments, the mode diameter of the antibodies or antigen binding fragments thereof is less than 400 nm, less than 350 nm, less than 300 nm, less than 250 nm, less than 200 nm, less than 150 nm, less than 100 nm, less than 90 nm, less than 80 nm, less than 70 nm, less than 60 nm, or less than 50 nm.

In some embodiments, the antibody has a tumor growth inhibition percentage (TGI %) that is greater than 10%, 20%, 30%, 40%, 50%, 60%, 70%, 80%, 90%, 100%, 110%, 120%, 130%, 140%, 150%, 160%, 170%, 180%, 190%, or 200%. In some embodiments, the antibody has a tumor growth inhibition percentage that is less than 60%, 70%, 80%, 90%, 100%, 110%, 120%, 130%, 140%, 150%, 160%, 170%, 180%, 190%, or 200%. The TGI % can be determined, e.g., at 3, 4, 5, 6, 7, 8, 9, 10, 11, 12, 13, 14, 15, 16, 17, 18, 19, 20, 21, 22, 23, 24, 25, 26, 27, 28, 29, or 30 days after the treatment starts, or 1, 2, 3, 4, 5, 6, 7, 8, 9, 10, 11, or 12 months after the treatment starts. As used herein, the tumor growth inhibition percentage (TGI %) is calculated using the following formula:

TGI(%)=[1−(Ti−T0)/(Vi−V0)]×100

Ti is the average tumor volume in the treatment group on day i. T0 is the average tumor volume in the treatment group on day zero. Vi is the average tumor volume in the control group on day i. V0 is the average tumor volume in the control group on day zero.

In some embodiments, the antibodies or antigen binding fragments have a functional Fc region. In some embodiments, effector function of a functional Fc region is antibody-dependent cell-mediated cytotoxicity (ADCC). In some embodiments, effector function of a functional Fc region is phagocytosis. In some embodiments, effector function of a functional Fc region is ADCC and phagocytosis. In some embodiments, the Fc region is human IgG1, human IgG2, human IgG3, or human IgG4. In some embodiments, the antibodies or antigen binding fragments do not have a functional Fc region. For example, the antibodies or antigen binding fragments are Fab, Fab′, F(ab′)₂, and Fv fragments.

### Methods of Making Anti-CitH3 Antibodies

An isolated fragment of human CitH3 can be used as an immunogen to generate antibodies using standard techniques for polyclonal and monoclonal antibody preparation. Polyclonal antibodies can be raised in animals by multiple injections (e.g., subcutaneous or intraperitoneal injections) of an antigenic peptide or protein. In some embodiments, the antigenic peptide or protein is injected with at least one adjuvant. In some embodiments, the antigenic peptide or protein can be conjugated to an agent that is immunogenic in the species to be immunized. Animals can be injected with the antigenic peptide or protein more than one time (e.g., twice, three times, or four times).

The full-length polypeptide or protein can be used or, alternatively, antigenic peptide fragments thereof can be used as immunogens. The antigenic peptide of a protein comprises at least 8 (e.g., at least 10, 15, 20, or 30) amino acid residues of the amino acid sequence of CitH3 and encompasses an epitope of the protein such that an antibody raised against the peptide forms a specific immune complex with the protein. As described above, the N-terminal sequence of human CitH3 is known in the art (SEQ ID NO: 22, CitH3 Amino Acid N-terminal Sequence: ACTKQTACKSTGGKAPCKQLATKAACKSAP).

An immunogen typically is used to prepare antibodies by immunizing a suitable subject (e.g., human or transgenic animal expressing at least one human immunoglobulin locus). An appropriate immunogenic preparation can contain, for example, a recombinantly expressed or a chemically-synthesized polypeptide (e.g., a fragment of human CitH3). The preparation can further include an adjuvant, such as Freund's complete or incomplete adjuvant, or a similar immunostimulatory agent.

Polyclonal antibodies can be prepared as described above by immunizing a suitable subject with a CitH3 polypeptide, or an antigenic peptide thereof (e.g., part of CitH3) as an immunogen. The antibody titer in the immunized subject can be monitored over time by standard techniques, such as with an enzyme-linked immunosorbent assay (ELISA) using the immobilized CitH3 polypeptide or peptide. If desired, the antibody molecules can be isolated from the mammal (e.g., from the blood) and further purified by well-known techniques, such as protein A of protein G chromatography to obtain the IgG fraction. At an appropriate time after immunization, e.g., when the specific antibody titers are highest, antibody-producing cells can be obtained from the subject and used to prepare monoclonal antibodies by standard techniques, such as the hybridoma technique or trioma techniques. The technology for producing hybridomas is well known in the art. Hybridoma cells producing a monoclonal antibody arc detected by screening the hybridoma culture supernatants for antibodies that bind the polypeptide or epitope of interest, e.g., using a standard ELISA assay.

Variants of the antibodies or antigen-binding fragments described herein can be prepared by introducing appropriate nucleotide changes into the DNA encoding a human, humanized, or chimeric antibody, or antigen-binding fragment thereof described herein, or by peptide synthesis. Such variants include, for example, deletions, insertions, or substitutions of residues within the amino acid sequences that make-up the antigen-binding site of the antibody or an antigen-binding domain. In a population of such variants, some antibodies or antigen-binding fragments will have increased affinity for the target protein, e.g., CitH3. Any combination of deletions, insertions, and/or combinations can be made to arrive at an antibody or antigen-binding fragment thereof that has increased binding affinity for the target. The amino acid changes introduced into the antibody or antigen-binding fragment can also alter or introduce new post-translational modifications into the antibody or antigen-binding fragment, such as changing (e.g., increasing or decreasing) the number of glycosylation sites, changing the type of glycosylation site (e.g., changing the amino acid sequence such that a different sugar is attached by enzymes present in a cell), or introducing new glycosylation sites.

Antibodies disclosed herein can be derived from any species of animal, including mammals. Non-limiting examples of native antibodies include antibodies derived from humans, primates, e.g., monkeys and apes, cows, pigs, horses, sheep, camelids (e.g., camels and llamas), chicken, goats, and rodents (e.g., rats, mice, hamsters and rabbits), including transgenic rodents genetically engineered to produce human antibodies.

Human and humanized antibodies include antibodies having variable and constant regions derived from (or having the same amino acid sequence as those derived from) human germline immunoglobulin sequences. Human antibodies may include amino acid residues not encoded by human germline immunoglobulin sequences (e.g., mutations introduced by random or site-specific mutagenesis in vitro or by somatic mutation in vivo), for example in the CDRs.

A humanized antibody typically has a human framework (FR) grafted with non-human CDRs. Thus, a humanized antibody has one or more amino acid sequence introduced into it from a source which is non-human. These non-human amino acid residues are often referred to as “import” residues, which are typically taken from an “import” variable domain. Humanization can be essentially performed by e.g., substituting rodent CDRs or CDR sequences for the corresponding sequences of a human antibody. Accordingly, “humanized” antibodies are chimeric antibodies wherein substantially less than an intact human V domain has been substituted by the corresponding sequence from a non-human species. In practice, humanized antibodies are typically mouse antibodies in which some CDR residues and some FR residues are substituted by residues from analogous sites in human antibodies.

The choice of human VH and VL domains to be used in making the humanized antibodies is very important for reducing immunogenicity. According to the so-called “best-fit” method, the sequence of the V domain of a mouse antibody is screened against the entire library of known human-domain sequences. The human sequence which is closest to that of the mouse is then accepted as the human FR for the humanized antibody.

It is further important that antibodies be humanized with retention of high specificity and affinity for the antigen and other favorable biological properties. To achieve this goal, humanized antibodies can be prepared by a process of analysis of the parental sequences and various conceptual humanized products using three-dimensional models of the parental and humanized sequences. Three-dimensional immunoglobulin models are commonly available and are familiar to those skilled in the art. Computer programs are available which illustrate and display probable three-dimensional conformational structures of selected candidate immunoglobulin sequences. Inspection of these displays permits analysis of the likely role of the residues in the functioning of the candidate immunoglobulin sequence, i.e., the analysis of residues that influence the ability of the candidate immunoglobulin to bind its antigen. In this way, FR residues can be selected and combined from the recipient and import sequences so that the desired antibody characteristic, such as increased affinity for the target antigen(s), is achieved.

Ordinarily, amino acid sequence variants of the human, humanized, or chimeric anti-CitH3 antibody will contain an amino acid sequence having at least 75%, 80%, 85%, 90%, 95%, 96%, 97%, 98%, or 99% percent identity with a sequence present in the light or heavy chain of the original antibody.

Identity or homology with respect to an original sequence is usually the percentage of amino acid residues present within the candidate sequence that are identical with a sequence present within the human, humanized, or chimeric anti-CitH3 antibody or fragment, after aligning the sequences and introducing gaps, if necessary, to achieve the maximum percent sequence identity, and not considering any conservative substitutions as part of the sequence identity.

Additional modifications to the anti-CitH3 antibodies or antigen-binding fragments can be made. For example, a cysteine residue(s) can be introduced into the Fc region, thereby allowing interchain disulfide bond formation in this region. The homodimeric antibody thus generated may have any increased half-life in vitro and/or in vivo. Homodimeric antibodies with increased half-life in vitro and/or in vivo can also be prepared using heterobifunctional cross-linkers. Alternatively, an antibody can be engineered which has dual Fc regions.

In some embodiments, a covalent modification can be made to the anti-CitH3 antibody or antigen-binding fragment thereof. These covalent modifications can be made by chemical or enzymatic synthesis, or by enzymatic or chemical cleavage. Other types of covalent modifications of the antibody or antibody fragment are introduced into the molecule by reacting targeted amino acid residues of the antibody or fragment with an organic derivatization agent that is capable of reacting with selected side chains or the N- or C-terminal residues.

In some embodiments, antibody variants are provided having a carbohydrate structure that lacks fucose attached (directly or indirectly) to an Fc region. For example, the amount of fucose in such an antibody may be from 1% to 80%, from 1% to 65%, from 5% to 65% or from 20% to 40%. The amount of fucose is determined by calculating the average amount of fucose within the sugar chain at Asn297, relative to the sum of all glycostructures attached to Asn 297 (e.g. complex, hybrid and high mannose structures) as measured by MALDI-TOF mass spectrometry, for example. Asn297 refers to the asparagine residue located at about position 297 in the Fc region (Eu numbering of Fc region residues; or position 314 in Kabat numbering); however, Asn297 may also be located about ±3 amino acids upstream or downstream of position 297, i.e., between positions 294 and 300, due to minor sequence variations in antibodies. Such fucosylation variants may have improved ADCC function. In some embodiments, to reduce glycan heterogeneity, the Fc region of the antibody can be further engineered to replace the Asparagine at position 297 with Alanine (N297A).

### Recombinant Vectors

The present disclosure also provides recombinant vectors (e.g., an expression vectors) that include an isolated polynucleotide disclosed herein (e.g., a polynucleotide that encodes a polypeptide disclosed herein), host cells into which are introduced the recombinant vectors (i.e., such that the host cells contain the polynucleotide and/or a vector comprising the polynucleotide), and the production of recombinant antibody polypeptides or fragments thereof by recombinant techniques.

As used herein, a “vector” is any construct capable of delivering one or more polynucleotide(s) of interest to a host cell when the vector is introduced to the host cell. An “expression vector” is capable of delivering and expressing the one or more polynucleotide(s) of interest as an encoded polypeptide in a host cell into which the expression vector has been introduced. Thus, in an expression vector, the polynucleotide of interest is positioned for expression in the vector by being operably linked with regulatory elements such as a promoter, enhancer, and/or a poly-A tail, either within the vector or in the genome of the host cell at or near or flanking the integration site of the polynucleotide of interest such that the polynucleotide of interest will be translated in the host cell introduced with the expression vector.

A vector can be introduced into the host cell by methods known in the art, e.g., electroporation, chemical transfection (e.g., DEAE-dextran), transformation, transfection, and infection and/or transduction (e.g., with recombinant virus). Thus, non-limiting examples of vectors include viral vectors (which can be used to generate recombinant virus), naked DNA or RNA, plasmids, cosmids, phage vectors, and DNA or RNA expression vectors associated with cationic condensing agents.

In some implementations, a polynucleotide disclosed herein (e.g., a polynucleotide that encodes a polypeptide disclosed herein) is introduced using a viral expression system (e.g., vaccinia or other pox virus, retrovirus, or adenovirus), which may involve the use of a non-pathogenic (defective), replication competent virus, or may use a replication defective virus. In the latter case, viral propagation generally will occur only in complementing virus packaging cells. Suitable systems are known in the art. Techniques for incorporating DNA into such expression systems are well known to those of ordinary skill in the art. The DNA may also be “naked,” and the uptake of naked DNA may be increased by coating the DNA onto biodegradable beads that are efficiently transported into the cells.

For expression, the DNA insert comprising an antibody-encoding or polypeptide-encoding polynucleotide disclosed herein can be operatively linked to an appropriate promoter (e.g., a heterologous promoter), such as the phage lambda PL promoter, the E. coli lac, trp and tac promoters, the SV40 early and late promoters and promoters of retroviral LTRs, to name a few. Other suitable promoters are known to the skilled artisan. The expression constructs can further contain sites for transcription initiation, termination and, in the transcribed region, a ribosome binding site for translation. The coding portion of the mature transcripts expressed by the constructs may include a translation initiating at the beginning and a termination codon (UAA, UGA, or UAG) appropriately positioned at the end of the polypeptide to be translated.

As indicated, the expression vectors can include at least one selectable marker. Such markers include dihydrofolate reductase or neomycin resistance for eukaryotic cell culture and tetracycline or ampicillin resistance genes for culturing in E. coli and other bacteria. Representative examples of appropriate hosts include, but are not limited to, bacterial cells, such as E. coli, Streptomyces, and Salmonella typhimurium cells; fungal cells, such as yeast cells; insect cells such as Drosophila S2 and Spodoptera Sf9 cells; animal cells such as CHO, COS, Bowes melanoma, and HK 293 cells; and plant cells. Appropriate culture mediums and conditions for the host cells described herein are known in the art.

Non-limiting vectors for use in bacteria include pQE70, pQE60 and pQE-9, available from Qiagen; pBS vectors, Phagescript vectors, Bluescript vectors, pNH8A, pNH16a, pNH18A, pNH46A, available from Stratagene; and ptrc99a, pKK223-3, pKK233-3, pDR540, pRIT5 available from Pharmacia. Non-limiting eukaryotic vectors include pWLNEO, pSV2CAT, pOG44, pXT1 and pSG available from Stratagene; and pSVK3, pBPV, pMSG and pSVL available from Pharmacia. Other suitable vectors will be readily apparent to the skilled artisan.

Non-limiting bacterial promoters suitable for use include the E. coli lacI and lacZ promoters, the T3 and T7 promoters, the gpt promoter, the lambda PR and PL promoters and the trp promoter. Suitable eukaryotic promoters include the CMV immediate early promoter, the HSV thymidine kinase promoter, the early and late SV40 promoters, the promoters of retroviral LTRs, such as those of the Rous sarcoma virus (RSV), and metallothionein promoters, such as the mouse metallothionein-I promoter.

In the yeast Saccharomyces cerevisiae, a number of vectors containing constitutive or inducible promoters such as alpha factor, alcohol oxidase, and PGH may be used.

Introduction of the construct into the host cell can be effected by calcium phosphate transfection, DEAE-dextran mediated transfection, cationic lipid-mediated transfection, electroporation, transduction, infection or other methods. Such methods are described in many standard laboratory manuals and are known in the art.

Transcription of DNA encoding an antibody of the present disclosure by higher eukaryotes may be increased by inserting an enhancer sequence into the vector. Enhancers are cis-acting elements of DNA, usually about from 10 to 300 bp that act to increase transcriptional activity of a promoter in a given host cell-type. Examples of enhancers include the SV40 enhancer, which is located on the late side of the replication origin at base pairs 100 to 270, the cytomegalovirus early promoter enhancer, the polyoma enhancer on the late side of the replication origin, and adenovirus enhancers.

For secretion of the translated protein into the lumen of the endoplasmic reticulum, into the periplasmic space or into the extracellular environment, appropriate secretion signals may be incorporated into the expressed polypeptide. The signals may be endogenous to the polypeptide, or they may be heterologous signals.

The polypeptide (e.g., antibody) can be expressed in a modified form, such as a fusion protein (e.g., a GST-fusion) or with a histidine-tag, and may include not only secretion signals, but also additional heterologous functional regions. For instance, a region of additional amino acids, particularly charged amino acids, may be added to the N-terminus of the polypeptide to improve stability and persistence in the host cell, during purification, or during subsequent handling and storage. Also, peptide moieties can be added to the polypeptide to facilitate purification. Such regions can be removed prior to final preparation of the polypeptide. The addition of peptide moieties to polypeptides to engender secretion or excretion, to improve stability and to facilitate purification, among others, are familiar and routine techniques in the art.

### Methods of Treatment

The antibodies or antibody or antigen-binding fragments thereof of the present disclosure can be used for various therapeutic purposes. In one aspect, the disclosure provides methods for treating immune-dysfunctions in a subject, methods of reducing symptoms of immune-dysfunctions in a subject, or methods of reducing the risk of developing immune-dysfunctions. In some embodiments, the treatment can halt, slow, retard, or inhibit progression of immune-dysfunction. In some embodiments, the treatment can result in the reduction of in the number, severity, and/or duration of one or more symptoms of immune-dysfunction in a subject. In some embodiments, the immune-dysfunction is caused by NET activation and release (NETosis). In some embodiments, the methods described herein can inhibit NETosis.

In one aspect, the disclosure provides methods for treating, preventing, or reducing the risk of developing disorders associated with an abnormal or unwanted immune response, e.g., immune response caused by infection or an autoimmune disorder, whereby the use of the anti-CitH3 antibody can improve immune function or can reduce NETosis/pyroptosis associated tissue injuries.

In some embodiments, these autoimmune disorders include, but are not limited to, Alopecia arcata, lupus, ankylosing spondylitis, Meniere's disease, antiphospholipid syndrome, mixed connective tissue disease, autoimmune Addison's disease, multiple sclerosis, autoimmune hemolytic anemia, myasthenia gravis, autoimmune hepatitis, pemphigus vulgaris, Behcet's disease, pernicious anemia, bullous pemphigoid, polyarthritis nodosa, cardiomyopathy, polychondritis, celiac sprue-dermatitis, polyglandular syndromes, chronic fatigue syndrome (CFIDS), polymyalgia rheumatica, chronic inflammatory demyelinating, polymyositis and dermatomyositis, chronic inflammatory polyneuropathy, primary agammaglobulinemia, Churg-Strauss syndrome, primary biliary cirrhosis, cicatricial pemphigoid, psoriasis, CREST syndrome, Raynaud's phenomenon, cold agglutinin disease, Reiter's syndrome, Crohn's disease, Rheumatic fever, discoid lupus, rheumatoid arthritis, Cryoglobulinemia sarcoidosis, fibromyalgia, scleroderma, Grave's disease, Sjögren's syndrome, Guillain-Barre, stiff-man syndrome, Hashimoto's thyroiditis, Takayasu arteritis, idiopathic pulmonary fibrosis, temporal arteritis/giant cell arteritis, idiopathic thrombocytopenia purpura (ITP), ulcerative colitis, IgA nephropathy, uveitis, diabetes (e.g., Type I), vasculitis, lichen planus, and vitiligo. The anti-CitH3 antibodies or antigen-binding fragments thereof can also be administered to a subject to treat, prevent, or reduce the risk of developing disorders associated with an abnormal or unwanted immune response associated with NETosis. In some embodiments, the subject has Crohn's disease, ulcerative colitis or type 1 diabetes. In some embodiments, the subject has autoimmune thyroid disease, Grave's disease, multiple sclerosis, psoriasis, inflammatory bowel disease (e.g., Crohn's Disease (CD) and ulcerative colitis), rheumatoid arthritis, Sjögren's syndrome, autoimmune nephritis, or systemic lupus erythematosus.

In some embodiments, the infection is caused by bacteria and/or viruses (e.g., COVID-19). In one aspect, the disclosure provides methods for treating, preventing, or reducing the risk of developing infectious diseases, or reducing or improving the symptoms associated with an infectious disease, e.g., sepsis or septic shock, that is caused by bacteria and/or viruses.

The antibodies or antibody or antigen-binding fragments thereof of the present disclosure can be used for various therapeutic purposes. In one aspect, the disclosure provides methods for treating a cancer in a subject, methods of reducing the rate of the increase of volume of a tumor in a subject over time, methods of reducing the risk of developing a metastasis, or methods of reducing the risk of developing an additional metastasis in a subject. In some embodiments, the treatment can halt, slow, retard, or inhibit progression of a cancer. In some embodiments, the treatment can result in the reduction of in the number, severity, and/or duration of one or more symptoms of the cancer in a subject.

As used herein, the term “cancer” refers to cells having the capacity for autonomous growth. Examples of such cells include cells having an abnormal state or condition characterized by rapidly proliferating cell growth. The term is meant to include cancerous growths, e.g., tumors; oncogenic processes, metastatic tissues, and malignantly transformed cells, tissues, or organs, irrespective of histopathologic type or stage of invasiveness. Also included are malignancies of the various organ systems, such as respiratory, cardiovascular, renal, reproductive, hematological, neurological, hepatic, gastrointestinal, and endocrine systems; as well as adenocarcinomas which include malignancies such as most colon cancers, renal-cell carcinoma, prostate cancer and/or testicular tumors, non-small cell carcinoma of the lung, and cancer of the small intestine. Cancer that is “naturally arising” includes any cancer that is not experimentally induced by implantation of cancer cells into a subject, and includes, for example, spontaneously arising cancer, cancer caused by exposure of a patient to a carcinogen(s), cancer resulting from insertion of a transgenic oncogene or knockout of a tumor suppressor gene, and cancer caused by infections, e.g., viral infections. The term “carcinoma” is art recognized and refers to malignancies of epithelial or endocrine tissues. The term also includes carcinosarcomas, which include malignant tumors composed of carcinomatous and sarcomatous tissues. An “adenocarcinoma” refers to a carcinoma derived from glandular tissue or in which the tumor cells form recognizable glandular structures. The term “sarcoma” is art recognized and refers to malignant tumors of mesenchymal derivation. The term “hematopoietic neoplastic disorders” includes diseases involving hyperplastic/neoplastic cells of hematopoietic origin. A hematopoietic neoplastic disorder can arise from myeloid, lymphoid or erythroid lineages, or precursor cells thereof.

In one aspect, the disclosure features methods that include administering a therapeutically effective amount of an antibody or antigen-binding fragment thereof disclosed herein to a subject in need thereof (e.g., a subject having, or identified or diagnosed as having, a cancer), e.g., breast cancer (e.g., triple-negative breast cancer), carcinoid cancer, cervical cancer, endometrial cancer, glioma, head and neck cancer, liver cancer, lung cancer, small cell lung cancer, lymphoma, melanoma, ovarian cancer, pancreatic cancer, prostate cancer, renal cancer, colorectal cancer, gastric cancer, testicular cancer, thyroid cancer, bladder cancer, urethral cancer, or hematologic malignancy. In some embodiments, the cancer is unresectable melanoma or metastatic melanoma, non-small cell lung carcinoma (NSCLC), small cell lung cancer (SCLC), bladder cancer, or metastatic hormone-refractory prostate cancer. In some embodiments, the subject has a solid tumor. In some embodiments, the cancer is squamous cell carcinoma of the head and neck (SCCHN), renal cell carcinoma (RCC), triple-negative breast cancer (TNBC), or colorectal carcinoma.

In one aspect, the disclosure features methods that include administering a therapeutically effective amount of an antibody or antigen-binding fragment thereof disclosed herein to a subject in need thereof (e.g., a subject having, or identified or diagnosed as having, a bacterial or viral infection, or reducing inflammation resulting from a bacterial or viral infection. In some aspects, the compositions and methods disclosed herein can be used for treatment of patients at risk of a viral or bacterial infection. Patients with viral or bacterial infections can be identified using various methods known in the art. In one aspect, the disclosure is related to methods of decreasing adverse effects due to viral or bacterial infection, such as lung injury, reduction in body weight, reduction in levels of inflammatory markers such as cytokines. Methods of treatment include administration of an effective amount of the antibody or antigen-binding fragment thereof disclosed herein, alone or in combination with one or more therapeutic agent or compound effective for such treatments, e.g. rhMG53, and others known in the art.

In some embodiments, the compositions and methods disclosed herein can be used for treatment of patients at risk for a cancer. Patients with cancer can be identified with various methods known in the art.

In one aspect, the disclosure is related to methods of decreasing the rate of tumor growth, including contacting a tumor cell with an effective amount of a composition including the antibody or antigen-binding fragment thereof, or the antibody-drug conjugate described herein. In one aspect, the disclosure is related to methods of killing a tumor cell, including contacting a tumor cell with an effective amount of a composition including the antibody or antigen-binding fragment thereof, or the antibody-drug conjugate described herein.

As used herein, by an “effective amount” is meant an amount or dosage sufficient to effect beneficial or desired results including halting, slowing, retarding, or inhibiting progression of a disease, e.g., an immuno-dysfunction and chronic wounds. An effective amount will vary depending upon, e.g., an age and a body weight of a subject to which the antibody, antigen binding fragment, antibody-encoding polynucleotide, vector comprising the polynucleotide, and/or compositions thereof is to be administered, a severity of symptoms and a route of administration, and thus administration can be determined on an individual basis.

An effective amount can be administered in one or more administrations. By way of example, an effective amount of an antibody or an antigen binding fragment is an amount sufficient to ameliorate, stop, stabilize, reverse, inhibit, slow and/or delay progression of a disease in a patient. As is understood in the art, an effective amount of an antibody or antigen binding fragment may vary, depending on, inter alia, patient history as well as other factors such as the type (and/or dosage) of antibody used.

Effective amounts and schedules for administering the antibodies, antibody-encoding polynucleotides, and/or compositions disclosed herein may be determined empirically, and making such determinations is within the skill in the art. Those skilled in the art will understand that the dosage that must be administered will vary depending on, for example, the mammal that will receive the antibodies, antibody-encoding polynucleotides, and/or compositions disclosed herein, the route of administration, the particular type of antibodies, antibody-encoding polynucleotides, antigen binding fragments, and/or compositions disclosed herein used and other drugs being administered to the mammal. Guidance in selecting appropriate doses for antibody or antigen binding fragment can be found in the literature on therapeutic uses of antibodies and antigen binding fragments.

A typical daily dosage of an effective amount of an antibody is 0.01 mg/kg to 100 mg/kg. In some embodiments, the dosage can be less than 100 mg/kg, 10 mg/kg, 9 mg/kg, 8 mg/kg, 7 mg/kg, 6 mg/kg, 5 mg/kg, 4 mg/kg, 3 mg/kg, 2 mg/kg, 1 mg/kg, 0.5 mg/kg, or 0.1 mg/kg. In some embodiments, the dosage can be greater than 10 mg/kg, 9 mg/kg, 8 mg/kg, 7 mg/kg, 6 mg/kg, 5 mg/kg, 4 mg/kg, 3 mg/kg, 2 mg/kg, 1 mg/kg, 0.5 mg/kg, 0.1 mg/kg, 0.05 mg/kg, or 0.01 mg/kg. In some embodiments, the dosage is about 10 mg/kg, 9 mg/kg, 8 mg/kg, 7 mg/kg, 6 mg/kg, 5 mg/kg, 4 mg/kg, 3 mg/kg, 2 mg/kg, 1 mg/kg, 0.9 mg/kg, 0.8 mg/kg, 0.7 mg/kg, 0.6 mg/kg, 0.5 mg/kg, 0.4 mg/kg, 0.3 mg/kg, 0.2 mg/kg, or 0.1 mg/kg.

In any of the methods described herein, the at least one antibody, antigen-binding fragment thereof, or pharmaceutical composition (e.g., any of the antibodies, antigen-binding fragments, or pharmaceutical compositions described herein) and, optionally, at least one additional therapeutic agent can be administered to the subject at least once a week (e.g., once a week, twice a week, three times a week, four times a week, once a day, twice a day, or three times a day). In some embodiments, at least two different antibodies and/or antigen-binding fragments are administered in the same composition (e.g., a liquid composition). In some embodiments, at least one antibody or antigen-binding fragment and at least one additional therapeutic agent are administered in the same composition (e.g., a liquid composition). In some embodiments, the at least one antibody or antigen-binding fragment and the at least one additional therapeutic agent are administered in two different compositions (e.g., a liquid composition containing at least one antibody or antigen-binding fragment and a solid oral composition containing at least one additional therapeutic agent). In some embodiments, the at least one additional therapeutic agent is administered as a pill, tablet, or capsule. In some embodiments, the at least one additional therapeutic agent is administered in a sustained-release oral formulation.

In some embodiments, the one or more additional therapeutic agents can be administered to the subject prior to, or after administering the at least one antibody, antigen-binding antibody fragment, or pharmaceutical composition (e.g., any of the antibodies, antigen-binding antibody fragments, or pharmaceutical compositions described herein). In some embodiments, the one or more additional therapeutic agents and the at least one antibody, antigen-binding antibody fragment, or pharmaceutical composition (e.g., any of the antibodies, antigen-binding antibody fragments, or pharmaceutical compositions described herein) are administered to the subject such that there is an overlap in the bioactive period of the one or more additional therapeutic agents and the at least one antibody or antigen-binding fragment (e.g., any of the antibodies or antigen-binding fragments described herein) in the subject.

In some embodiments, the subject can be administered the at least one antibody, antigen-binding antibody fragment, or pharmaceutical composition (e.g., any of the antibodies, antigen-binding antibody fragments, or pharmaceutical compositions described herein) over an extended period of time (e.g., over a period of at least 1 week, 2 weeks, 3 weeks, 1 month, 2 months, 3 months, 4 months, 5 months, 6 months, 7 months, 8 months, 9 months, 10 months, 11 months, 12 months, 1 year, 2 years, 3 years, 4 years, or 5 years). A skilled medical professional may determine the length of the treatment period using any of the methods described herein for diagnosing or following the effectiveness of treatment (e.g., the observation of at least one symptom of a disease, e.g., immuno-dysfunction). As described herein, a skilled medical professional can also change the identity and number (e.g., increase or decrease) of antibodies or antigen-binding antibody fragments (and/or one or more additional therapeutic agents) administered to the subject and can also adjust (e.g., increase or decrease) the dosage or frequency of administration of at least one antibody or antigen-binding antibody fragment (and/or one or more additional therapeutic agents) to the subject based on an assessment of the effectiveness of the treatment (e.g., using any of the methods described herein and known in the art).

### Pharmaceutical Compositions and Routes of Administration

Also provided herein are pharmaceutical compositions that contain at least one (e.g., one, two, three, or four) of the antibodies or antigen-binding fragments described herein. Two or more (e.g., two, three, or four) of any of the antibodies or antigen-binding fragments described herein can be present in a pharmaceutical composition in any combination. The pharmaceutical compositions may be formulated in any manner known in the art.

Pharmaceutical compositions are formulated to be compatible with their intended route of administration (e.g., intravenous, intraarterial, intramuscular, intradermal, subcutaneous, or intraperitoneal). The compositions can include a sterile diluent (e.g., sterile water or saline), a fixed oil, polyethylene glycol, glycerine, propylene glycol or other synthetic solvents, antibacterial or antifungal agents, such as benzyl alcohol or methyl parabens, chlorobutanol, phenol, ascorbic acid, thimerosal, and the like, antioxidants, such as ascorbic acid or sodium bisulfite, chelating agents, such as ethylenediaminetetraacetic acid, buffers, such as acetates, citrates, or phosphates, and isotonic agents, such as sugars (e.g., dextrose), polyalcohols (e.g., mannitol or sorbitol), or salts (e.g., sodium chloride), or any combination thereof. Liposomal suspensions can also be used as pharmaceutically acceptable carriers. Preparations of the compositions can be formulated and enclosed in ampules, disposable syringes, or multiple dose vials. Where required (as in, for example, injectable formulations), proper fluidity can be maintained by, for example, the use of a coating, such as lecithin, or a surfactant. Absorption of the antibody or antigen-binding fragment thereof can be prolonged by including an agent that delays absorption (e.g., aluminum monostearate and gelatin). Alternatively, controlled release can be achieved by implants and microencapsulated delivery systems, which can include biodegradable, biocompatible polymers (e.g., ethylene vinyl acetate, polyanhydrides, polyglycolic acid, collagen, polyorthoesters, and polylactic acid; Alza® Corporation and Nova Pharmaceutical, Inc.).

Compositions containing one or more of any of the antibodies or antigen-binding fragments described herein can be formulated for parenteral (e.g., intravenous, intraarterial, intramuscular, intradermal, subcutaneous, or intraperitoneal) administration in dosage unit form (i.e., physically discrete units containing a predetermined quantity of active compound for ease of administration and uniformity of dosage).

Toxicity and therapeutic efficacy of compositions can be determined by standard pharmaceutical procedures in cell cultures or experimental animals (e.g., monkeys). One can, for example, determine the LD50 (the dose lethal to 50% of the population) and the ED50 (the dose therapeutically effective in 50% of the population): the therapeutic index being the ratio of LD50:ED50. Agents that exhibit high therapeutic indices are preferred. Where an agent exhibits an undesirable side effect, care should be taken to minimize potential damage (i.e., reduce unwanted side effects). Toxicity and therapeutic efficacy can be determined by other standard pharmaceutical procedures.

Data obtained from cell culture assays and animal studies can be used in formulating an appropriate dosage of any given agent for use in a subject (e.g., a human). A therapeutically effective amount of the one or more (e.g., one, two, three, or four) antibodies or antigen-binding fragments thereof (e.g., any of the antibodies or antibody fragments described herein) will be an amount that treats the disease in a subject in a subject, or a subject identified as being at risk of developing the disease, decreases the severity, frequency, and/or duration of one or more symptoms of a disease in a subject (e.g., a human). The effectiveness and dosing of any of the antibodies or antigen-binding fragments described herein can be determined by a health care professional or veterinary professional using methods known in the art, as well as by the observation of one or more symptoms of disease in a subject (e.g., a human). Certain factors may influence the dosage and timing required to effectively treat a subject (e.g., the severity of the disease or disorder, previous treatments, the general health and/or age of the subject, and the presence of other diseases).

Exemplary doses include milligram or microgram amounts of any of the antibodies or antigen-binding fragments described herein per kilogram of the subject's weight (e.g., about 1 μg/kg to about 500 mg/kg; about 100 μg/kg to about 500 mg/kg; about 100 μg/kg to about 50 mg/kg; about 10 μg/kg to about 5 mg/kg; about 10 μg/kg to about 0.5 mg/kg; or about 1 μg/kg to about 50 μg/kg). While these doses cover a broad range, one of ordinary skill in the art will understand that therapeutic agents, including antibodies and antigen-binding fragments thereof, vary in their potency, and effective amounts can be determined by methods known in the art. Typically, relatively low doses are administered at first, and the attending health care professional or veterinary professional (in the case of therapeutic application) or a researcher (when still working at the development stage) can subsequently and gradually increase the dose until an appropriate response is obtained. In addition, it is understood that the specific dose level for any particular subject will depend upon a variety of factors including the activity of the specific compound employed, the age, body weight, general health, gender, and diet of the subject, the time of administration, the route of administration, the rate of excretion, and the half-life of the antibody or antibody fragment in vivo.

The pharmaceutical compositions can be included in a container, pack, or dispenser together with instructions for administration. The disclosure also provides methods of manufacturing the antibodies or antigen binding fragments thereof for various uses as described herein.

### EXAMPLES

The invention is further described in the following examples, which do not limit the scope of the invention described in the claims.

### Example 1: Generating Mouse Anti-CitH3 Antibodies

To generate mouse antibodies against human CitH3 (SEQ ID NO: 22), BALB/c mice were immunized with human CitH3 protein. Spleen tissues of the immunized mice were isolated, and spleen cells were fused with myeloma cells to generate hybridomas. Screening of the hybridomas was performed and those producing high-titer mouse antibodies were subject to sequencing. Specifically, a lead mouse anti-CitH3 antibody Y1128 was selected for humanization.

### Example 2: Humanization of the Mouse Antibodies

The starting point for humanization was the mouse antibody Y1128. The amino acid sequences for the heavy chain variable region (VH) and the light chain variable region (VL) of these mouse antibodies were determined as SEQ ID NO: 14

### (EVQVQQSGAELVRPGALVKLSCKASGFNIKDYYMHWMKQRPEQGLEWIGWIDPENGN TIYDPKFQGKATITSDTSSNTAYLQLNSLTSEDTAVYYCAPFGNYVWFAYWGQGTLVTV SA) and SEQ ID NO: 15,

(DVLMTQTPLSLPVSLGDQASISCRSSQSIVHSNGITYLEWYLQKPGQSPKLLIYKVSNRF SGVPDRFSGSGSGTDFILKISRVEAEDLGLYYCFQGSHVPFTFGSGTKLEIKR) respectively. A chimeric antibody with the same VH and VL of Y1128 was constructed, which contains human heavy chain constant regions (e.g., CH1, CH2, and CH3 domains) and light chain constant region (e.g., CL domain).

During the humanization process, mouse CDRs were grafted into the human framework acceptor, and residues in human framework which are different from those in murine framework were studied. Backmutations from human residues to mouse residues were designed.

Four humanized heavy chain variable region variants (SEQ ID NOs: 7-10) and three humanized light chain variable region variants (SEQ ID NOs: 11-13) for Y1128 were constructed, containing different modifications or substitutions.

These humanized heavy chain variable region variants can be combined with any of the light chain variable region variants derived from the same mouse antibody. For example, Y1128-VH1 (SEQ ID NO: 7) can be combined with any humanized light chain variable region variant based on the same mouse antibody Y1128 (e.g., SEQ ID NOs: 11-13), and the antibody will be labeled accordingly. For example, if Y1128-VH1 is combined with Y1128-VL3 (SEQ ID NO: 13), the antibody is labeled as Y1128-VH1/VL3 (or VH1/VL3).

### Example 3: Initial Binding Screening of Humanized Antibody Candidates by Elisa

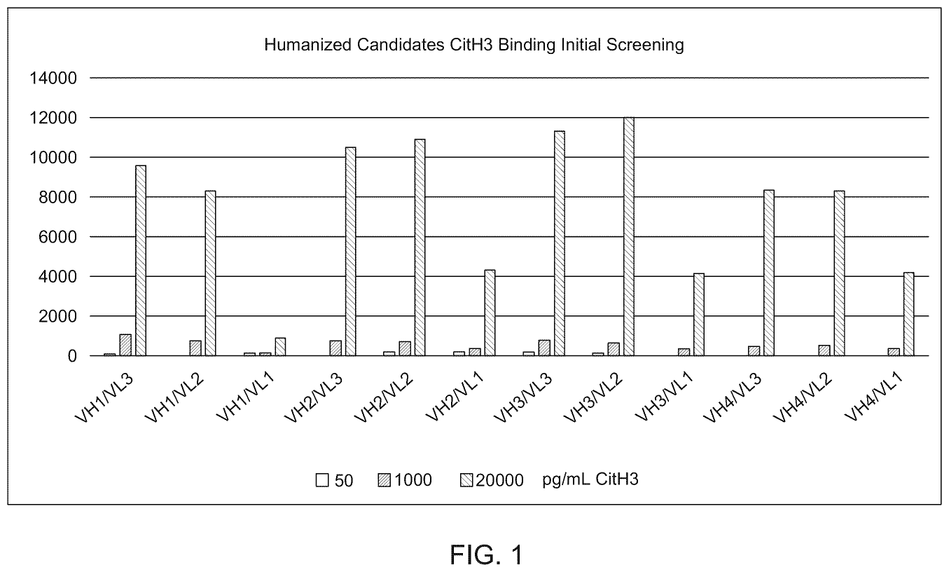

The binding affinity of the humanized anti-CitH3 antibodies against CitH3 was screened by ELISA. Specifically, a high-binding ELISA plate was coated with 100 μL test antibodies at 1 μg/mL in phosphate-buffered saline (PBS). The plate was covered and incubated at 4° C. overnight. On the next day, the plate was washed three times with 1×PBS/0.05% Tween® buffer (0.05% PBST), rotated, and the wash cycle was repeated for a total of 6 times. Each subsequent wash was performed in the same manner. The plate was hit on a towel to dry wells after the last wash. Afterwards, the ELISA plate was blocked with 150 μL/well blocking buffer (1% bovine serum albumin (BSA) in 0.05% PBST) at room temperature (RT) on a plate shaker (covered) for 1 hour. After blocking, the plate was washed six times and 100 μL of each peptide (serially diluted in blocking buffer) was added. The plate was incubated at RT for 1 hour on a plate shaker. After the incubation, the plate was washed six times, and 100 μL/well rabbit anti-Histone H3 (citrulline R2+R8+R17) secondary antibody (Abcam®, Cambridge, MA, Cat #: ab5103) in blocking buffer at 1:3000 dilution ratio was added. The plate was then incubated at RT for 1 hour on a plate shaker. After the incubation, the plate was washed six times, and 100 μL/well goat anti-rabbit IgG (H&L) HRP secondary antibody (Jackson ImmunoResearch, West Grove, PA, Cat #: 111-035-144) in blocking buffer at 1:10000 dilution ratio was added. The plate was incubated at RT for 1 hour on a plate shaker. After the incubation, the plate was washed six times, and 100 μL/well of Amplex™ M Red development substrate was added. The plate was kept in a dark drawer for 15 minutes, and then analyzed in a plate reader with the following parameters: Excitation (Ex) 530, Emission (Em) 590, and Cutoff 570 nm. The results are shown in the table below and FIG. 1.

TABLE 4
ELISA RESULTS (RAW DATA) OF HUMANIZED
ANTIBODIES BINDING TO CITH3

| CitH3 (pg/mL) | VH1/VL3 | VH1/VL2 | VH1/VL1 | VH2/VL3 | VH2/VL2 | VH2/VL1 |
|---|---|---|---|---|---|---|
| 20000 | 12462.001 | 10156.92 | 3697.806 | 13406.188 | 13339.729 | 6860.002 |
| 1000 | 3936.653 | 2601.407 | 2912.86 | 3638.259 | 3154.382 | 2912.843 |
| 50 | 2917.288 | 1869.583 | 2948.313 | 2862.777 | 2591.251 | 2705.122 |
| 0 | 2834.645 | 1821.894 | 2776.461 | 2822.027 | 2394.932 | 2517.558 |
| CitH3 (pg/mL) | VH3/VL3 | VH3/VL2 | VH3/VL1 | VH4/VL3 | VH4/VL2 | VH4/VL1 |
| 20000 | 14253.509 | 15094.391 | 7148.555 | 11395.56 | 11009.893 | 7228.581 |
| 1000 | 3640.412 | 3763.073 | 3391.827 | 3457.085 | 3206.184 | 3341.961 |
| 50 | 3047.626 | 3188.234 | 2725.887 | 2873.642 | 2603.029 | 2876.707 |
| 0 | 2865.457 | 3060.969 | 2982.573 | 2980.21 | 2643.849 | 2967.142 |

As shown in FIG. 1, the initial screening ELISA results showed strong signals from multiple variants when CitH3 was added at 20000 pg/ml. Humanized antibodies VH1/VL3 (VH: SEQ ID NO: 7, VL: SEQ ID NO: 13), VH2/VL3 (VH: SEQ ID NO: 8, VL: SEQ ID NO: 13), VH2/VL2 (VH: SEQ ID NO: 8, VL: SEQ ID NO: 12), VH3/VL3 (VH: SEQ ID NO: 9, VL: SEQ ID NO: 13), and VH3/VL2 (VH: SEQ ID NO: 9, VL: SEQ ID NO: 12) showed the strongest response and were selected for subsequent experiments.

### Example 4: Heat Treatment and Binding of Humanized Antibody Candidates by Elisa

The effect of heat treatment of selected anti-CitH3 antibodies was determined by measuring the binding affinity to CitH3. ELISA assays were performed using the chimeric antibody (VH: SEQ ID NO: 14, VL: SEQ ID NO: 15) or humanized antibodies to assess their thermostability.

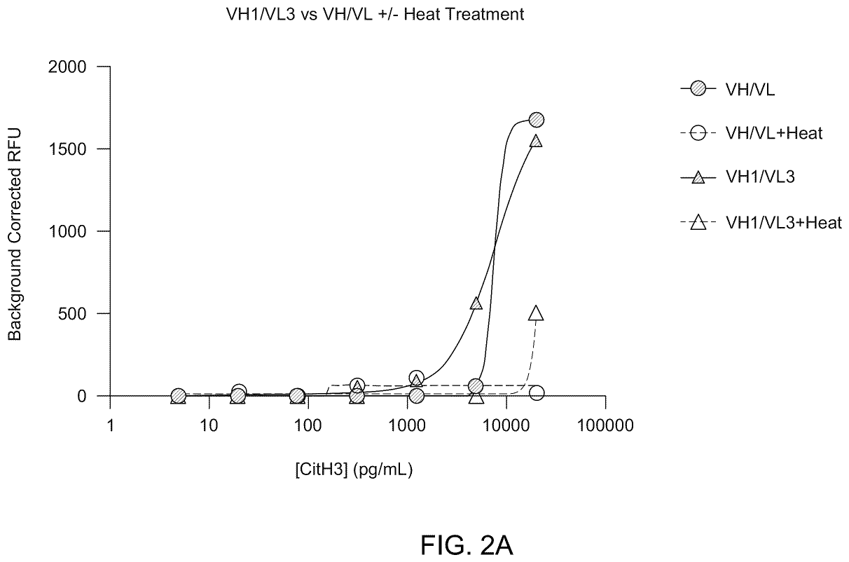

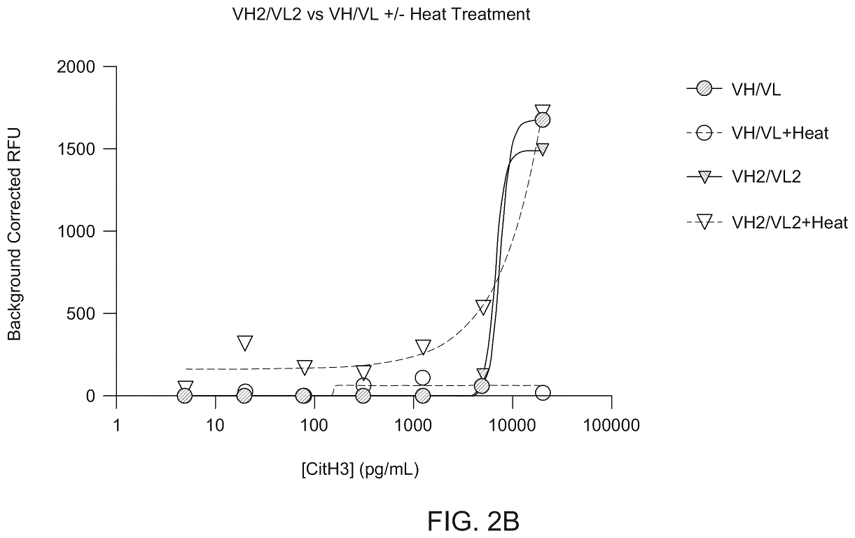

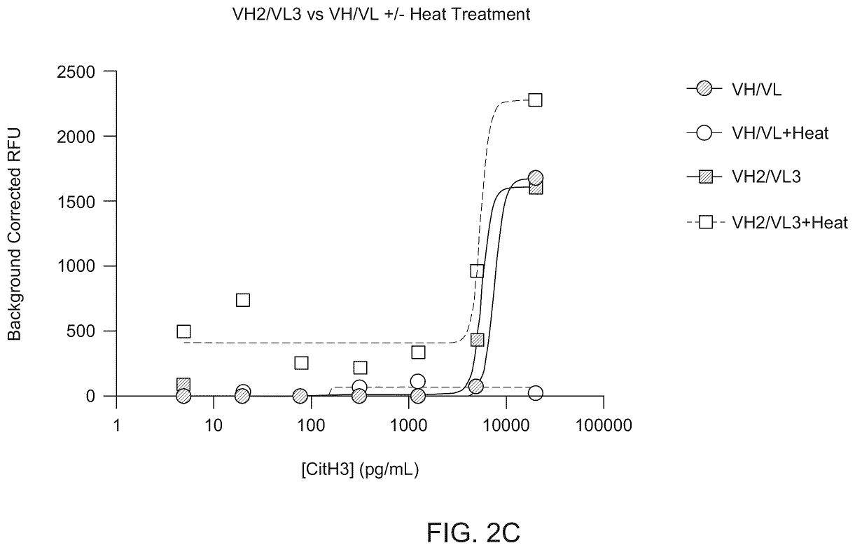

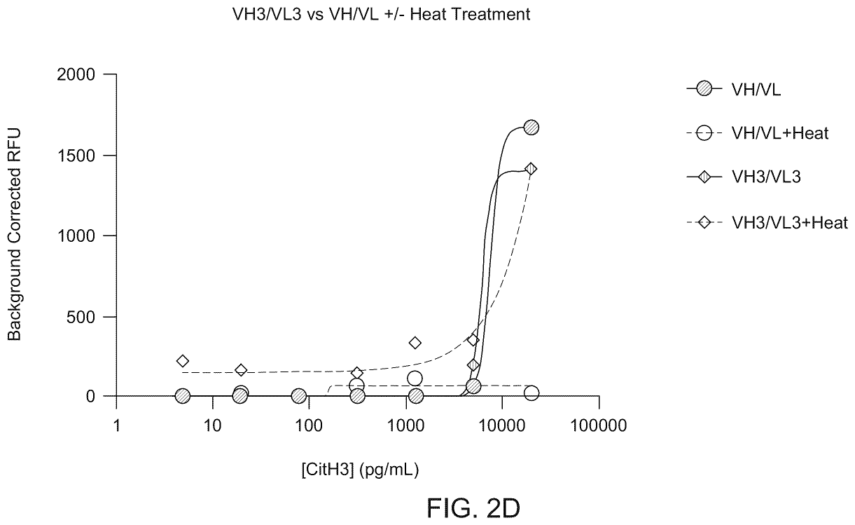

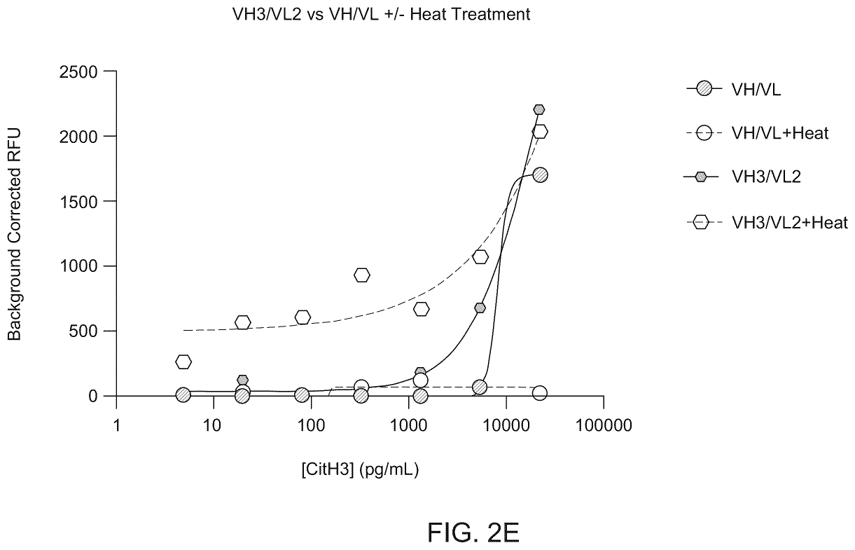

Specifically, a high-binding ELISA plate was coated with 100 μL goat anti-human IgG Fc antibody (Jackson ImmunoResearch, Cat #: 109-005-008) at 5 μg/mL in PBS. The plate was covered and incubated at 4° C. overnight. On the next day, the plate was washed three times with 1×PBS/0.05% Tween® buffer (0.05% PBST), rotated, and the wash cycle was repeated for a total of 6 times. Each subsequent wash was performed in the same manner. The plate was hit on a towel to dry wells after the last wash. Afterwards, the ELISA plate was blocked with 150 μL/well blocking buffer (1% BSA in 0.05% PBST) at RT on a plate shaker (covered) for 1 hour. After blocking, the plate was washed six times and 100 μL of each diluted antibody was added. Prior to loading, 50 μL of the diluted antibody was heated to 70° C. for 5 minutes by a PCR machine. The plate was incubated at RT for 1 hour on a plate shaker. After the incubation, the plate was washed six times, and 100 μL/well of each peptide (serially diluted) was added. The plate was then incubated at RT for 1 hour on a plate shaker. After the incubation, the plate was washed six times, and 100 μL/well rabbit anti-Histone H3 (citrulline R2+R8+R17) secondary antibody (Abcam®, Cat #: ab5103) in blocking buffer at 1:3000 dilution ratio was added. The plate was then incubated at RT for 1 hour on a plate shaker. After the incubation, the plate was washed six times, and 100 μL/well of goat anti-rabbit IgG (H&L) HRP secondary antibody (Jackson ImmunoResearch, Cat #: 111-035-144) in blocking buffer at 1:10000 dilution ratio was added. The plate was incubated at RT for 1 hour on a plate shaker. After the incubation, the plate was washed six times, and 100 μL/well of Amplex™ Red development substrate was added. The plate was kept in a dark drawer for 15 minutes, and then analyzed in a plate reader with the following parameters: Ex 530, Em 590, and Cutoff 570 nm. The results are shown in the table below and FIGS. 2A-2E.

TABLE 5
ELISA RESULTS (RAW DATA) OF HUMANIZED ANTIBODIES
BINDING TO CITH3 (+/−HEAT)

| CitH3 (pg/mL) | VH/VL | VH1/VL3 | VH2/VL3 | VH2/VL2 | VH3/VL3 | VH3/VL2 |
|---|---|---|---|---|---|---|
| | Untreated Ab | | | | | |
| 20000 | 4074.298 | 4727.629 | 4878.615 | 4318.171 | 4947.479 | 6112.12 |
| 5000 | 2459.449 | 3743.141 | 3699.841 | 2955.792 | 3743.034 | 4609.718 |
| 1250 | 2184.526 | 3249.073 | 3179.991 | 2482.853 | 3488.827 | 4115.418 |
| 312.5 | 2095.684 | 3240.281 | 3145.615 | 2495.037 | 3022.376 | 3955.764 |
| 78.13 | 2195.309 | 3203.371 | 3231.719 | 2598.368 | 3011.549 | 3862.2 |
| 19.53 | 1734.972 | 3166.793 | 3211.112 | 2591.676 | 3172.334 | 4057.852 |
| 4.88 | 2180.719 | 3150.939 | 3361.01 | 2681.316 | 3277.679 | 3916.099 |
| 0 | 2401.019 | 3177.64 | 3273.885 | 2828.678 | 3547.347 | 3947.033 |
| | 70° C. Heat Treated Ab | | | | | |
| 20000 | 2466.533 | 7351.222 | 5557.789 | 4149.942 | 5097.863 | 6372.393 |
| 5000 | 2509.039 | 6822.734 | 4239.575 | 2968.877 | 4034.44 | 5419.828 |
| 1250 | 2559.54 | 6941.382 | 3614.754 | 2726.001 | 4011.46 | 5023.378 |
| 312.5 | 2515.975 | 6381.478 | 3494.4 | 2562.178 | 3824.463 | 5282.706 |
| 78.13 | 2426.123 | 6485.267 | 3530.597 | 2592.637 | 3465.838 | 4958.807 |
| 19.53 | 2473.249 | 6568.989 | 4017.14 | 2744.227 | 3847.462 | 4921.273 |
| 4.88 | 2433.712 | 6318.603 | 3779.164 | 2467.683 | 3901.017 | 4622.12 |
| 0 | 2447.802 | 6846.381 | 3282.148 | 2427.33 | 3682.246 | 4371.28 |

As shown in FIGS. 2A-2E, humanized antibodies VH2/VL3, VH2/VL2, and VH3/VL3 showed no CitH3 binding loss after heat treatment, indicating a robust binding and thermostability under heat stress. By contrast, VH1/VL3 and VH3/VL2 showed decreases in CitH3 binding after heat treatment. As a result, these antibodies were not selected for subsequent experiments.

### Example 5: Non-Specific Baculovirus Binding of Humanized Lead Candidates by ELISA

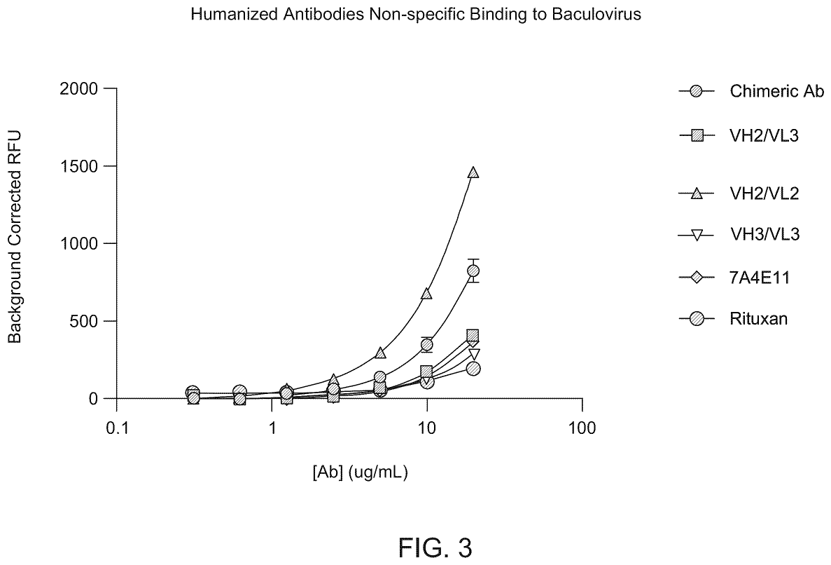

The non-specific binding to baculovirus of selected anti-CitH3 antibodies were determined by ELISA. Specifically, a high-binding ELISA plate was coated with 100 μL baculovirus diluted at 1:500 in PBS. The plate was covered and incubated at 4° C. overnight. On the next day, the plate was washed three times with 1×PBS/0.05% Tween® buffer (0.05% PBST), rotated, and the wash cycle was repeated for a total of 6 times. Each subsequent wash was performed in the same manner. The plate was hit on a towel to dry wells after the last wash. Afterwards, the ELISA plate was blocked with 150 μL/well blocking buffer (1% BSA in 0.05% PBST) at RT on a plate shaker (covered) for 1 hour. After blocking, the plate was washed six times and 100 μL of each diluted antibody was added. The plate was incubated at RT for 1 hour on a plate shaker. After the incubation, the plate was washed six times, and 100 μL/well goat anti-human IgG Fc HRP secondary antibody (Jackson ImmunoResearch, Cat #: 109-035-098), or goat anti-mouse IgG Fc HRP (Jackson ImmunoResearch, Cat #: 115-035-008) in blocking buffer at 1:5000 dilution ratio was added. The plate was then incubated at RT for 1 hour on a plate shaker. After the incubation, the plate was washed six times, and 100 μL/well of Amplex™ Red development substrate was added. The plate was kept in a dark drawer for 15 minutes, and then analyzed in a plate reader with the following parameters: Ex 530, Em 590, and Cutoff 570 nm. The results are shown in the table below and FIG. 3.

TABLE 6
ELISA RESULTS (RAW DATA) OF HUMANIZED ANTIBODIES BINDING TO BACULOVIRUS

| Ab | | | | | | | | | | | | |
|---|---|---|---|---|---|---|---|---|---|---|---|---|
| (μg/mL) | Chimeric Ab | | VH2/VL3 | | VH2/VL2 | | VH3/VL3 | | 7A4E11 | | Rituxan | |
| 20 | 925.31 | 821.52 | 459.77 | 454.1 | 1513.4 | 1516.6 | 329.09 | 350.9 | 402.78 | 415.36 | 234.4 | 250.18 |
| 10 | 430.98 | 362.46 | 216.75 | 222.27 | 714.61 | 753.94 | 176.07 | 168.69 | 189.79 | 196 | 161.82 | 164.83 |
| 5 | 215.79 | 160.57 | 116.76 | 110.65 | 341.55 | 352.48 | 116.15 | 114.8 | 98.613 | 102.33 | 108.64 | 107.67 |
| 2.5 | 117.91 | 91.403 | 69.524 | 71.069 | 172.94 | 187.03 | 74.854 | 81.869 | 58.685 | 65.532 | 93.5 | 104.8 |
| 1.25 | 89.779 | 59.007 | 54.613 | 55.287 | 96.471 | 105.96 | 57.278 | 70.478 | 43.484 | 45.098 | 78.133 | 87.639 |
| 0.625 | 54.844 | 41.468 | 43.694 | 44.426 | 65.598 | 74.295 | 58.286 | 53.715 | 37.033 | 36.647 | 82.181 | 89.279 |
| 0.313 | 54.843 | 44.502 | 78.003 | 44.054 | 51.977 | 55.179 | 45.004 | 57.483 | 31.036 | 35.054 | 80.028 | 82.323 |
| 0 | 54.38 | 39.721 | 43.448 | 41.252 | 43.797 | 43.331 | 46.063 | 53.85 | 27.661 | 28.568 | 71.218 | 85.314 |

As shown in FIG. 3, humanized antibodies VH2/VL3 and VH3/VL3 showed a similar low binding capacity to baculovirus as compared to negative controls (7A4E11 and Rituxan (a clinically approved anti-CD20 monoclonal antibody drug)), indicating that the humanization did not increase their nonspecific binding. However, humanized antibody VH2/VL2 and the chimeric antibody (VH/VL) both showed a higher binding signal as compared to negative controls, indicating an increased capacity for nonspecific binding.

### Example 6: Final Binding Characterization of Humanized Lead Candidates by ELISA

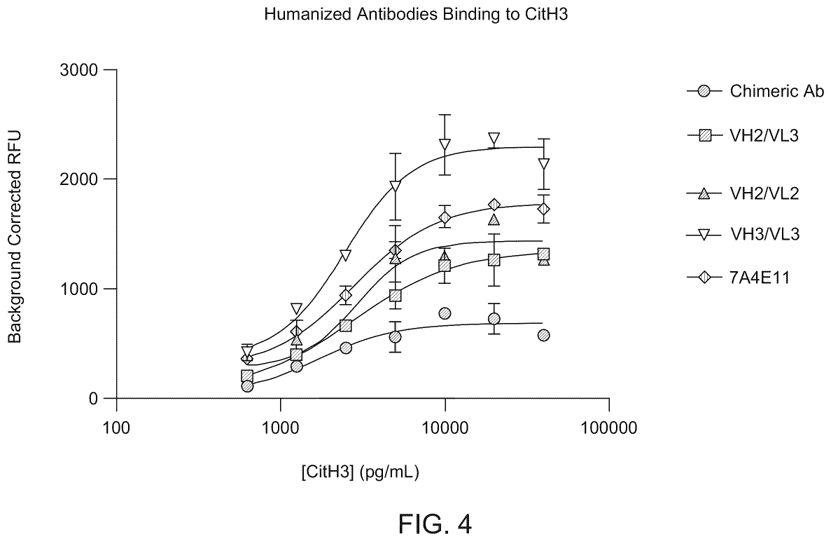

The binding affinity of the humanized lead anti-CitH3 antibodies against CitH3 was verified by ELISA. The same method as described in Example 3 was used. The results are shown in the table below and FIG. 4.

TABLE 7
ELISA RESULTS (RAW DATA) OF HUMANIZED ANTIBODIES BINDING TO CITH3

| CitH3 | | | | | | | | | | |
|---|---|---|---|---|---|---|---|---|---|---|
| (pg/mL) | Chimeric Ab | | VH2/VL3 | | VH2/VL2 | | VH3/VL3 | | 7A4E11 | |
| 40000 | 667.921 | 647.55 | 1394.528 | 1431.75 | 1313.159 | 1390.346 | 2362.083 | 2039.527 | 1859.428 | 1677.059 |
| 20000 | 902.124 | 706.274 | 1191.033 | 1526.711 | 1755.005 | 1686.896 | 2378.437 | 2478.944 | 1837.58 | 1779.523 |
| 10000 | 882.905 | 836.607 | 1416.459 | 1193.042 | 1327.557 | 1430.679 | 2580.198 | 2186.971 | 1772.286 | 1628.393 |
| 5000 | 740.621 | 537.669 | 1121.495 | 946.299 | 1152.718 | 1573.174 | 2207.39 | 1783.014 | 1448.413 | 1337.408 |
| 2500 | 509.598 | 571.924 | 760.835 | 757.724 | 705.639 | 758.116 | 1392.652 | 1331.529 | 1039.699 | 916.858 |
| 1250 | 355.789 | 383.504 | 536.878 | 452.495 | 742.385 | 495.327 | 856.558 | 904.227 | 671.915 | 623.281 |
| 625 | 215.553 | 173.742 | 337.088 | 264.848 | 315.474 | 270.335 | 538.469 | 432.636 | 406.923 | 396.568 |
| 0 | 83.411 | 84.382 | 100.608 | 94.25 | 87.789 | 75.489 | 70.581 | 66.881 | 43.782 | 42.506 |

As shown in FIG. 4, humanized antibody lead VH3/VL3 showed higher binding signal as compared to 7A4E11, while VH2/VL3 and VH2/VL2 showed roughly equivalent binding signal as compared to 7A4E11. In addition, the chimeric Antibody (VH/VL) showed the lowest binding signal of the antibodies tested. Strong signals from the humanized lead antibodies demonstrate that humanization did not significantly impact binding to CitH3.

### Example 7: Determination of Antibody Purity by SDS-PAGE

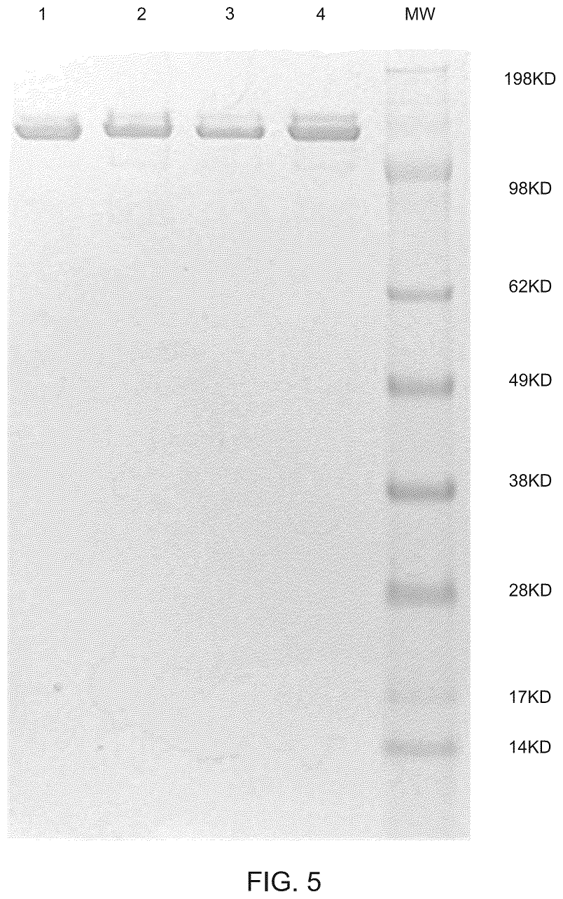

The purified antibodies, including the chimeric antibody (VH/VL) and three humanized antibodies (VH2/VL2, VH2/VL3, and VH3/VL3) were subject to sodium dodecyl sulfate-polyacrylamide gel electrophoresis (SDS-PAGE) analysis. 1 μg of each protein was loaded. As shown in FIG. 5, all the antibodies showed a single major band, indicating a high purity.

### Example 8: Determination of Thermostability by DSF/SLS

Immunoglobulin G (IgG) has a multi-domain structure, and each domain has its own melting temperature (Tm). For example, CH2 domain usually has a Tm of about 70° C. in PBS, and CH3 domain is more stable, with a Tm of about 80° C. The fragment antigen-binding region (Fab region) can have a wide Tm range, e.g., about 50-85° C., due to sequence variations. Therefore, the Tm value measured by various analytical techniques is usually an “apparent” transition temperature, rather than the real Tm for each domain. While this DSF assay can generate more than one Tm value, only Tm1 matters when thermostability is evaluated for antibody therapeutics. Tagg is the temperature at which SLS starts to detect aggregation. Tagg 266 measures SLS at 266 nm, which is more sensitive and suitable to detect smaller aggregation particles. Tagg 473 measures SLS at 473 nm and is better to detect larger aggregation particles. The chimeric antibody (VH/VL) and three humanized antibodies (VH2/VL2, VH2/VL3, and VH3/VL3) were subject to differential scanning fluorimetry (DSF) and static light scattering (SLS) analysis to determine their thermostability. Specifically, each sample was submitted to the UNcle system (Unchained Labs) for analysis. A temperature ramp of 1.3° C./min was performed with monitoring from 25° C. to 95° C. for DSF and SLS. UNcle was used to measure thermos aggregation at 266 nm and 473 nm by SLS. Melting temperature™ and aggregation temperature (Tagg) were calculated and analyzed using UNcle Analysis Software.

TABLE 8
DSF/SLS RESULTS OF SELECTED ANTIBODIES

| | DSF (° C.) | | | | | | SLS (° C.) | | | |
|---|---|---|---|---|---|---|---|---|---|---|
| | | Tm1 | | Tm2 | | Tm3 | Tagg | Tagg | Tagg | Tagg |
| Sample | Tm1 | SD | Tm2 | SD | Tm3 | SD | 266 | 266 SD | 473 | 473 SD |
| Chimeric Ab | 68.6 | 0.34 | N/A | N/A | N/A | N/A | 68.9 | 0.49 | 70.1 | 0.46 |
| VH2/VL2 | 69.9 | 0.35 | 82.2 | 2.69 | N/A | N/A | 71.4 | 0.39 | 72.5 | 0.21 |
| VH2/VL3 | 68.6 | 0.16 | 78.0 | 0.65 | N/A | N/A | 70.8 | 0.13 | 71.4 | 0.01 |
| VH3/VL3 | 68.3 | 0.31 | 83.1 | 0.19 | N/A | N/A | 69.9 | 0.26 | 71.3 | 0.12 |
| 7A4E11 | 68.8 | 1.64 | N/A | N/A | N/A | N/A | 64.8 | 1.50 | 68.8 | 1.31 |

As shown in the summary table above, the DSF and SLS results showed the mean and standard deviation of triplicated sample measurements. The DSF/SLS results indicate that all three humanized antibody candidates have similar melting temperature (Tm1) and thermal aggregation risk as compared to the chimeric antibody.

### Example 9: Determination of Aggregation by DLS

Dynamic light scattering (DLS) was used to detect aggregation in the antibody sample. Specifically, DLS was performed at 25° C. on UNcle (Unchained Labs) immediately prior to DSF/SLS. UNcle Analysis Software was used for data calculation and analysis.

TABLE 9
DLS RESULTS OF SELECTED ANTIBODIES

| | Peak 1 | | | | | Peak 2 | |
|---|---|---|---|---|---|---|---|
| | Mode | Mode | | | | Mode | |
| | Diameter | Diameter SD | Mass | | PDI | Diameter | Mass |
| Sample | (nm) | (nm) | (%) | PDI | SD | (nm) | (%) |
| Chimeric Ab | 10.937 | 1.042 | 99.97% | 0.275 | 0.004 | 67.09 | <0.1% |
| VH2/VL2 | 11.560 | 1.444 | 99.45% | 0.268 | 0.020 | 95.91 | 0.53% |
| VH2/VL3 | 10.067 | 0.456 | 99.97% | 0.215 | 0.031 | 325.7 | <0.1% |
| VH3/VL3 | 10.623 | 0.499 | 99.39% | 0.236 | 0.038 | 62.15 | 0.61% |
| 7A4E11 | 10.067 | 0.456 | 99.98% | 0.185 | 0.084 | 334.8 | <0.1% |

Typically, in DLS, “mode diameter” is protein particle diameter; and “mass percentage” is the amount of each size fraction in percentage. “PDI” is Polydispersity Index. Usually, the higher this index, the more polydispersity the sample is. If PDI is not larger than 0.25, the sample can be considered as monodisperse (Unchained Lab tech note). The DLS results showed the mean and standard deviation (when significant) of these sample measurements in triplicate. Each sample measurement is itself an average of eight sequential acquisitions.

As shown in the summary table above, all five samples contained antibodies of a typical diameter with insignificant to undetectable levels of aggregation at RT. The humanized antibodies VH3/VL3 and VH2/VL3 were monodisperse, whereas VH2/VL2 and the chimeric antibody showed limited polydispersity.

### Example 10: Analysis of Heterogeneity by Capillary Electrophoresis (CE)

The purified sample of VH3/VL3 (with highest expression level), together with the standard IgG were prepared in reducing and non-reducing labeling buffer before being submitted to the CE analysis. “IS” is a 10 kD protein serving as an internal standard. The results of the reducing-CE-SDS and non-reducing-CE-SDS are shown in the tables below.

TABLE 10
SUMMARY OF REDUCING-CE-SDS RESULTS

| | | PK#1 Relative | PK#2 | PK#2 Relative | PK#3 | PK#3 Relative |
|---|---|---|---|---|---|---|
| Sample ID | PK#1 (%) | Migration Time | (%) | Migration Time | (%) | Migration Time |
| IgG Std | 32.220 | 1.2180 | 7.0300 | 1.5310 | 60.750 | 1.5800 |
| VH3/VL3 | 30.340 | 1.2560 | 69.660 | 1.6340 | — | — |

TABLE 11
SUMMARY OF NON-REDUCING-CE-SDS RESULTS

| | | PK#1 | | PK#2 | | PK#3 | | PK#4 |
|---|---|---|---|---|---|---|---|---|
| | | Relative | | Relative | | Relative | | Relative |
| Sample | PK#1 | Migration | PK#2 | Migration | PK#3 | Migration | PK#4 | Migration |
| ID | (%) | Time | (%) | Time | (%) | Time | (%) | Time |
| IgG Std | 0.79000 | 2.0520 | 0.78000 | 2.2080 | 11.070 | 2.2560 | 87.360 | 2.3060 |
| VH3/VL3 | 1.6300 | 2.2640 | 98.370 | 2.3960 | — | — | — | — |

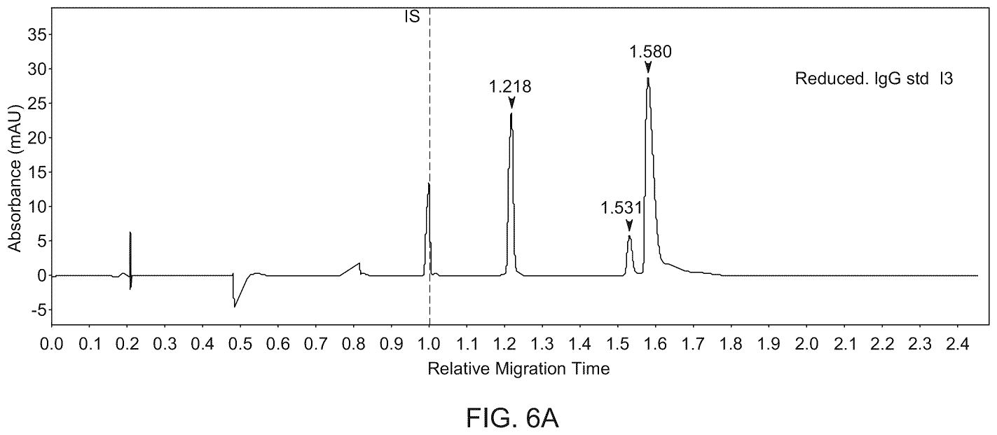

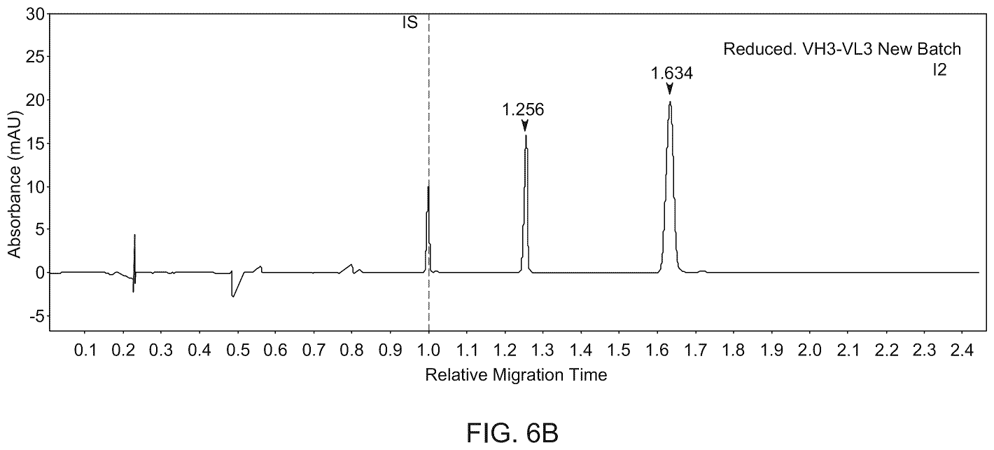

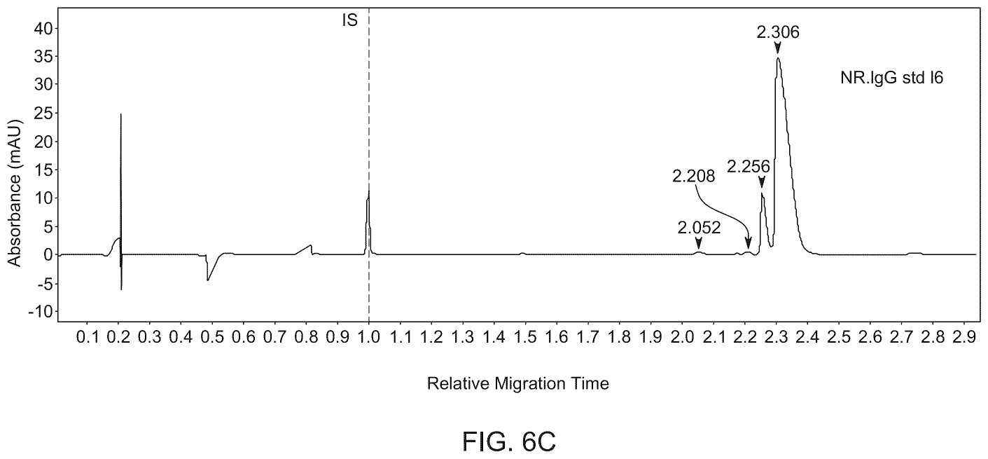

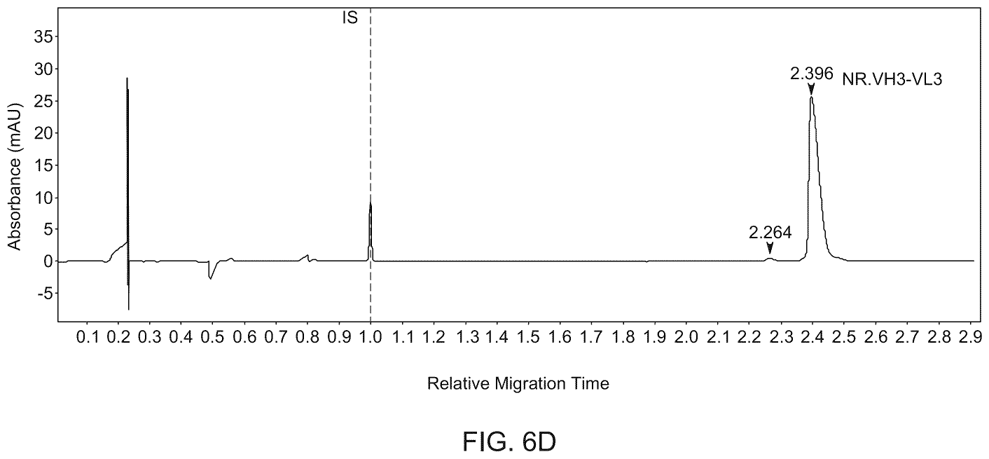

In the electrophoretogram for the reduced VH3/VL3 sample (FIG. 6B), the peak at 1.256 relative migration time (RMT) corresponds to the peak at 1.218 RMT in that of the reduced IgG standard sample (FIG. 6A), which is from IgG light chain. The peak at 1.634 RMT in the electrophoretogram of the reduced VH3/VL3 sample (FIG. 6B) corresponds to the peak at 1.580 RMT in that of the reduced IgG standard sample (FIG. 6A), which is from IgG heavy chain. No other species were observed in the VH3/VL3 electrophoretogram, suggesting high purity of the sample with no molecular weight (MW) variants.

In the electrophoretogram for the non-reduced VH3/VL3 sample (FIG. 6D), the peak observed at 2.396 RMT in the non-reduced VH3/VL3 electrophoretogram is 98.37% of the whole mass, corresponds to the peak at 2.306 in that of the non-reduced IgG standard sample (FIG. 6A), which is from intact IgG, suggesting high purity of the intact VH3/VL3 antibody sample. The very small peak in the non-reduced VH3/VL3 (FIG. 6D) at 2.264 RMT (1.6% total area) corresponds to the peak at 2.208 RMT observed in that of the non-reduced IgG sample (FIG. 6C), which should be from IgG missing one light chain.

As a conclusion, VH3/VL3, hereafter hCitH3-mAb, was selected as the final lead antibody based on its desired characterization and developability analysis results.

### Example 11: Comparative Analysis of Cayman CitH3-mAb and hCitH3-mAb Binding Affinity

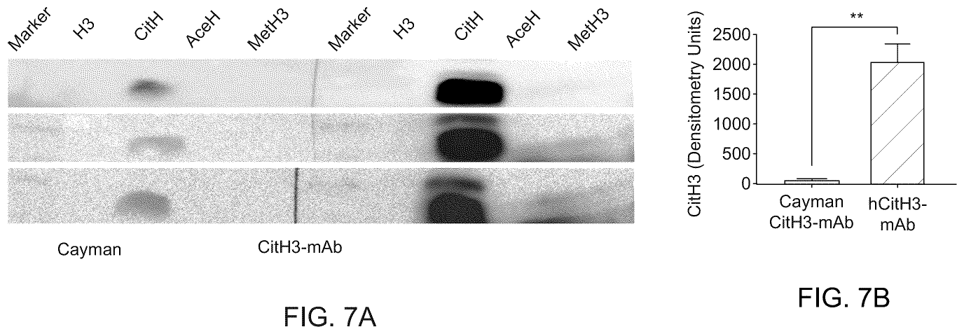

Four distinct peptides—histone H3 (H3), citrullinated histone H3 (CitH3; 4 Cit), acetylated histone H3 (AccH3), and methylated histone H3 (MetH3)—at a concentration of 0.5 microgram each, were run in duplicate through SDS-polyacrylamide gel electrophoresis and then transferred to a nitrocellulose membrane (FIG. 7A). This membrane was split into two sections for parallel probing with an equal quantity of Cayman CitH3-mAb (Ann Arbor, MI) or hCitH3-mAb (2 μg/ml) overnight. Post three extensive washes, the sections were incubated with HRP-conjugated secondary antibodies specific to mouse IgG and human IgG (Jackson ImmunoResearch Labs. Cat #309-035-008), respectively, for 2 hours. For concurrent signal development, both membrane sections were exposed simultaneously for the same duration. Quantitative analysis of the signal intensity was performed using Image Studio Lite, and the results are displayed as mean±SEM for three independent experiments (n=3/group). **: P<0.01 (FIG. 7B).

As expected, hCitH3-mAb possess superior binding affinity to the commercially available Cayman CitH3-mAb (FIG. 7B). As shown in FIG. 7A, hCitH3-mAb maintains high-specificity in recognizing CitH3 as antigen, but not unmodified H3, acetylated H3, or methylated H3. More importantly, hCitH3-mAb contains >100-fold binding capacity over the commercially available Cayman CitH3-mAb (FIGS. 7A-B).

### Example 12: Use of hCitH3-mAb as Therapeutic to Treat Sepsis-Induced Lung Injury and Multi-Organ Failure

### Methods

Mouse Studies—C57BL/6J mice aged 8-12 months were sourced from Jackson Laboratory, Bar Harbor, ME. Under isoflurane anesthesia, mice were intranasally infected with 2.5×10⁶ colony-forming unit (CFU) of Pseudomonas aeruginosa (strain PA 19660) for the septic shock model, adhering to the University of Michigan's Institutional Animal Care and Use Committee protocols. In the LPS-induced endotoxic shock model, LPS was administered intraperitoneally at 25 mg/kg. For the CLP-induced septic shock model, cecal ligation at 75% from the tip and puncture with a 21-gauge needle were performed. Post model induction, mice were treated with hCitH3-mAb or IgG. Survival was monitored over a 10-day period.

Western blots—Cell and tissue lysates were prepared using RIPA buffer (10 mM Tris-HCl, pH 7.2, 150 mM NaCl, 1% NP-40, 0.5% SDS, 0.5% sodium deoxycholate), with added protease inhibitors (Sigma-Aldrich®, St. Louis, MO) and phosphatase inhibitors (Thermo Fisher Scientific, Waltham, MA), for Western blot analysis. Lysates were separated by SDS-PAGE and electro-transferred onto nitrocellulose membranes. Membranes were washed with TBST, blocked with 5% milk in TBST for 1 hour, and probed with specific antibodies as detailed in the figures. Detection was performed using the Pierce™ ECL Plus kit.

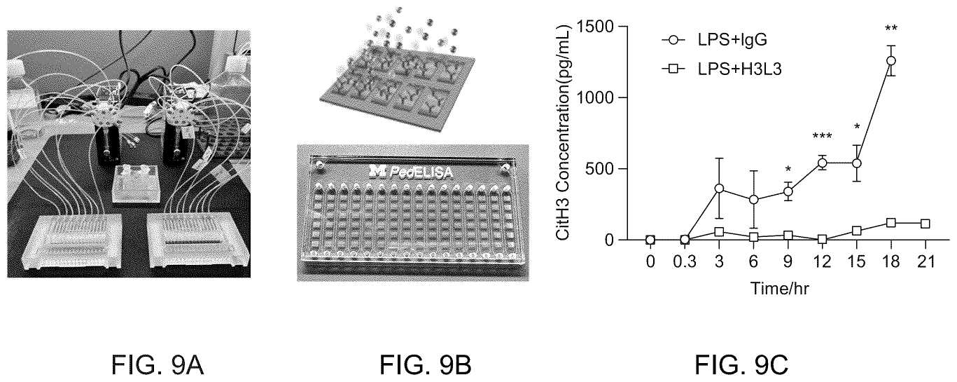

PEdELISA—The ‘pre-equilibrium digital ELISA’ (PEdELISA) allows for rapid quantification of protein biomarkers, requiring only 10 mL of serum with an incubation time between 15 to 300 seconds across a four-log dynamic range. In our study, plasma samples were collected at 3-hour intervals for CitH3 and cytokine levels analysis using PEdELISA (FIGS. 9A-9C). The PEd-ELISA microarray platform combines ultrasensitive single-molecule digital counting and transient biosensing for CitH3 quantification in whole blood. This technology offers rapid (<20 min) and highly sensitive detection of CitH3, allowing for real-time monitoring. Its high multiplexity capacity (up to 16-plex) and machine learning-based data analysis make it an invaluable tool for early diagnosis of microbial infections, timely administration of therapeutic interventions, and the potential to prevent sepsis-ALI progression.

Using this platform, an elevation in CitH3 can be detected as early as 3 hours following endotoxic shock. Most notably, administration of hCitH3-mAb effectively blocks CitH3 elevation and the development of cytokine storms. These findings suggest a potential CitH3 elevation threshold that can serve as a diagnostic biomarker for sepsis onset and a critical time window for administering therapeutic interventions.

Acute lung injury—Histological analysis of lung tissue was performed per our previous study. Briefly, the lung tissue was embedded in paraffin, sliced into 5-μm sections, and stained with hematoxylin and cosin (H&E). A pathologist, blinded to group allocation of the specimen, evaluated the sections.

Bacterial load determination—Homogenized tissues of lung and spleen in PBS (1 mL) and samples were serially diluted by 10-fold in sterile PBS. Ten microliters of each sample were plated on nutrient agar plates and incubated at 37° C. for 15 hours. The numbers of bacterial colonies were counted from the plates.

During systemic microbial infections, activated neutrophils undergo a unique cell death process, releasing DNA and cellular proteins, including elastase and histone H3, leading to the formation of “neutrophil extracellular traps (NETs).” Emerging evidence suggests that uncontrolled excessive NET formation contributes to sepsis-induced acute lung injury (ALI).

Citrullination of histone H3 (CitH3), catalyzed by peptidylarginine deiminases (PAD2 and PAD4), plays a crucial role in initiating NETs-induced immune cell death and tissue injury during severe infections. Sepsis-induced acute lung injury (ALI) and multi-organ failure poses substantial threats to public health, requiring prolonged hospitalization, often in intensive care units, resulting in significant strains in health care systems. The complex nature of ALI/ARDS, along with potential complications of multi-organ failure, makes the management and treatment of ALI patients extremely challenging.

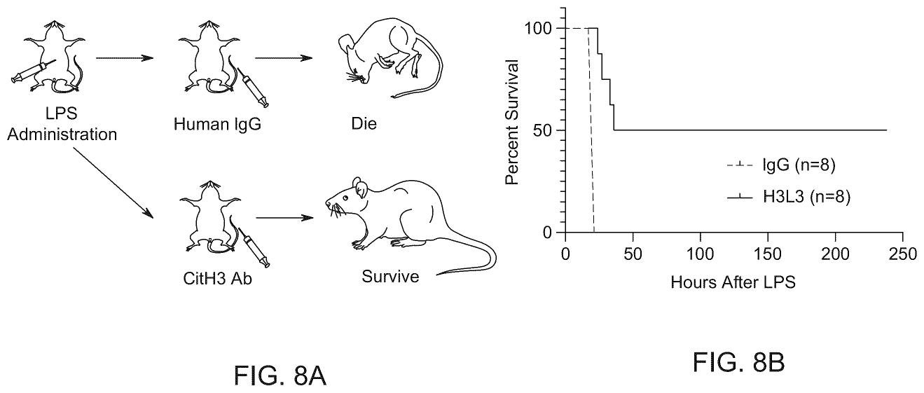

As shown in FIG. 8B, hCitH3-mAb improves survival of mice subjected to LPS-induced septic shock. C57BL6/J mice which were exposed to a lethal LPS dose (25 mg/kg, i.p) that caused complete mortality within 24 hours (with IgG as a control). Tail vein administration of hCitH3-mAb (20 mg/kg, tail vein) significantly improved survival.

Using the PEd-ELISA microarray platform (FIG. 9A-9B), an elevation in CitH3 can be detected as early as 3 hours following endotoxic shock (FIG. 9C). Most notably, administration of hCitH3-mAb effectively blocks CitH3 elevation and the development of cytokine storms. These findings suggest a potential CitH3 elevation threshold that can serve as a diagnostic biomarker for sepsis onset and a critical time window for administering therapeutic interventions.

### Example 13: hCitH3-mAb (H3L3) Improves Survival of Mice Subjected to Pseudomonas Aeruginosa (PA)-Induced Sepsis

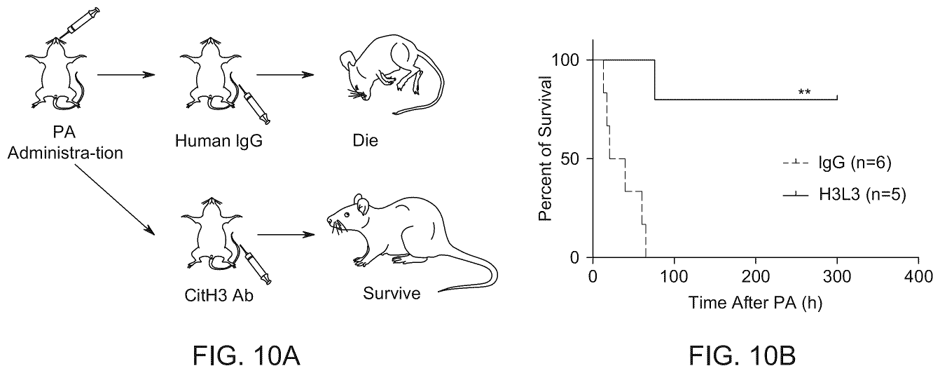

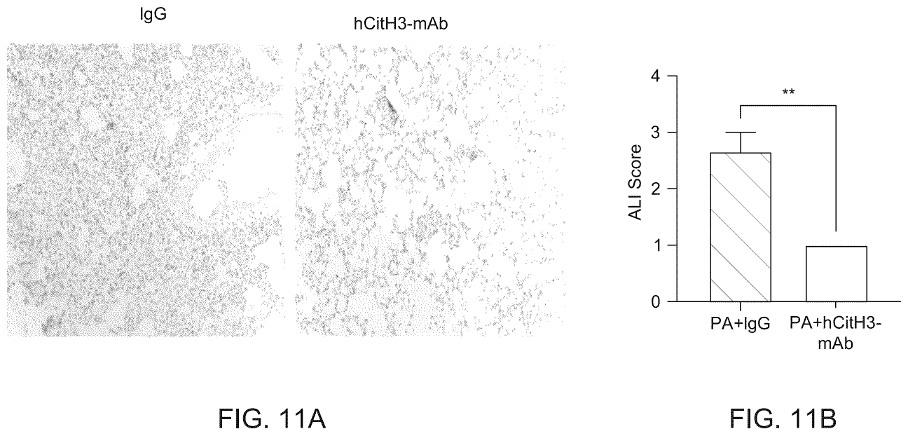

Studies were conducted to evaluate the effects of hCitH3-mAb in C57BL6/J mice (10-12 weeks, male) subjected to PA-induced septic shock (2.5×10⁶ CFU, intranasal). hCitH3-mAb (20 mg/kg) were delivered to mice via tail vein injection at 30 min post PA-administration. As control, mice received similar treatment of human IgG (Sigma). As shown in FIG. 10B, mice receiving the human IgG all died within 3 days (n=6), whereas about 80% of those receiving hCitH3-mAb survived (n=5). For histological evaluation of PA-induced ALI, separate experiments were conducted with mice subjected to PA-septic shock and sacrificed at 24 hours post hCitH3-mAb administration (n=5 for hCitH3-mAb, n=5 for IgG). Score of acute lung injury (ALI) was evaluated using mouse lung sections by a certified pathologist who was blinded to the animal experiment (FIG. 11A). As shown in FIG. 11A and FIG. 11B, mice receiving hCitH3-mAb showed significant reduction of pulmonary injury.

### Example 14: Humanized CitH3 Monoclonal Antibody (hCitH3-mAb) Improves Survival of Young Mice Subjected to CLP Sepsis

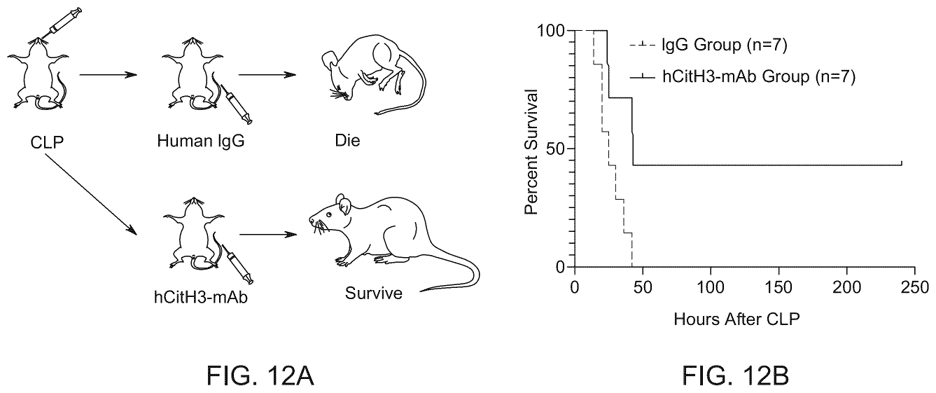

FIG. 12B shows survival curves for C57BL/6 mice (12 weeks of age) treated with or without hCitH3-mAb in a mouse model of the cecal ligation and puncture (CLP) induced lethal sepsis. C57BL/6 mice were administered a single dose of hCitH3-mAb (20 mg/kg of body weight) or human IgG (20 mg/kg of body weight) 0.5 hour before CLP. Survival rates were monitored for 10 days (n=7 per group). The results showed that hCitH3-mAb significantly improved mouse survival compared to the IgG group.

### Example 15: Evaluating hCitH3-mAb's Role in Bacterial Clearance During Septic Shock Induced by Pseudomonas Aeruginosa

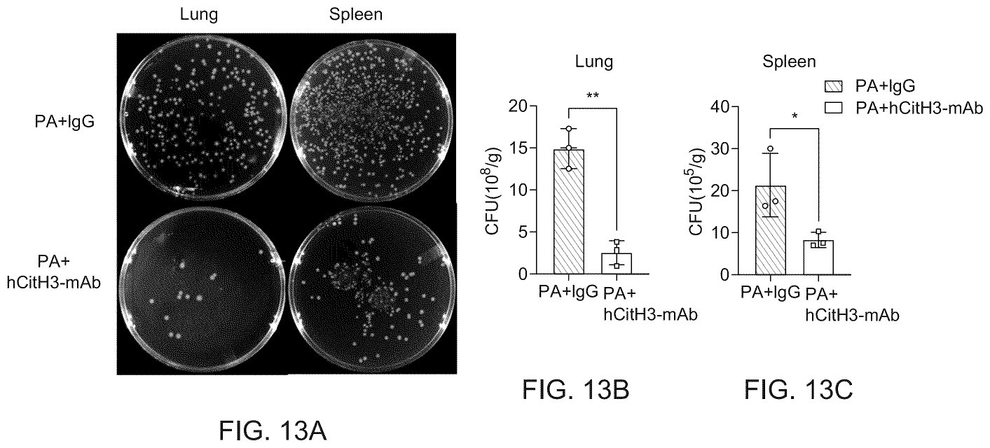

FIG. 13 shows assessment of bacterial load in lung and spleen of mice intranasally inoculated with 2.5×10⁶ Pseudomonas aeruginosa cells and then administered a tail vein injection of hCitH3-mAb (20 mg/kg) or human IgG (20 mg/kg). Post-infection, lung and spleen tissues were harvested and homogenized for bacterial load assessment by culturing on LB agar plates.

### Example 16: hCitH3-mAb can be Used to Treat Sepsis Multi-Organ Injury in Aging

### Methods

Aging C57BL/6J mice (male, 24-26 moths) were obtained from the National Institute of Aging. Mice were established under anesthesia with isoflurane according to protocols approved by IACUC at University of Virginia. Mouse sepsis was performed with Cecal Ligation and Puncture (CLP) model. Mice ceca were isolated out of the peritoneal cavity with sterile tweezers, ligated with 4-0 silk sutures, and punctured using a 20 G needle at the midpoint between the tail end and the ligation set.

Mice were divided into four groups: CLP, CLP treated with rhMG53, CLP treated with hCitH3-mAb, and CLP treated with rhMG53+hCitH3-mAb. hCitH3-mAb were administrated via tail vein injection with a dose of 20 mg/kg and recombinant human MG53 (rhMG53) protein was administered via intraperitoneal injection with a dose of 5 mg/kg after CLP.

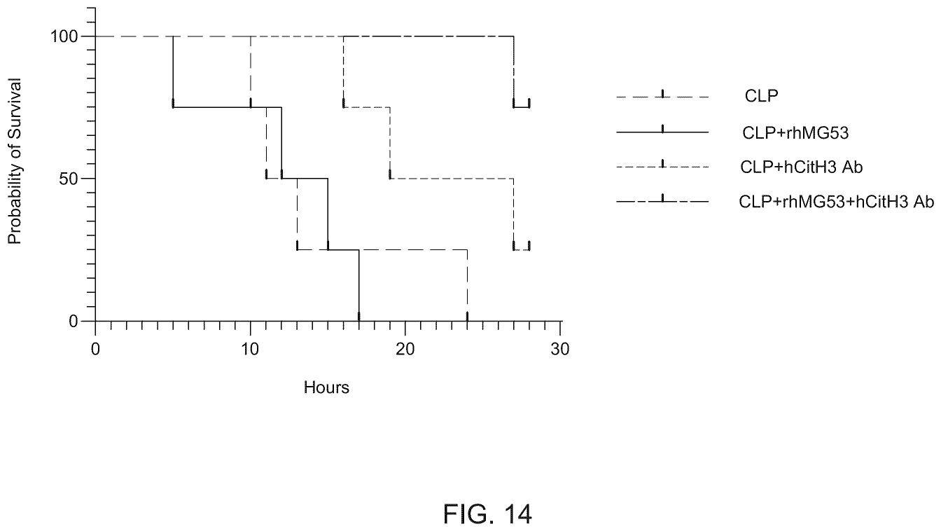

Results demonstrated that hCitH3-mAb treatment improved survival of aged mice subjected to CLP-sepsis (FIG. 14). Moreover, co-treatment with hCitH3-mAb and rhMG53 synergistically improved survival of aged mice following CLP.

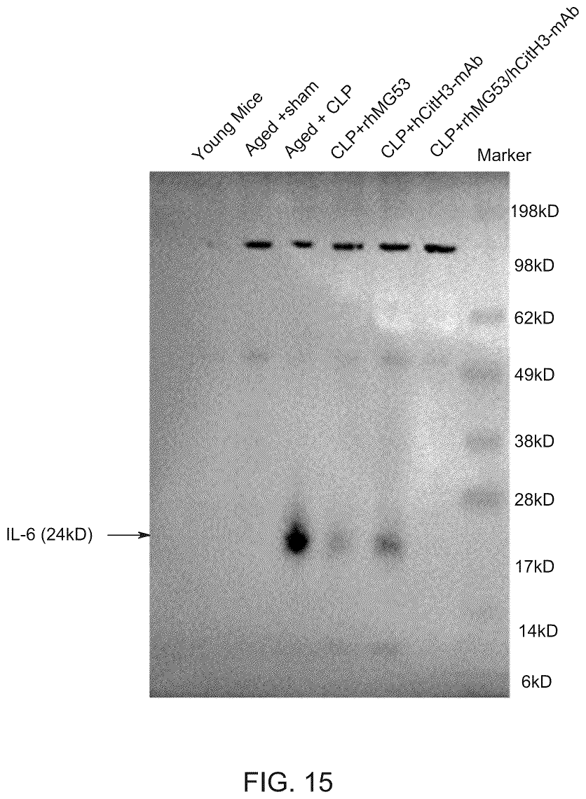

Lung tissues were collected from aged C57BL6 mice (25 months age) at 24-hour post CLP. WB were conducted using anti-IL-6 antibody (Cat #12912T, Cell Signaling Technology®, Danvers, MA). Referring to FIG. 15, compared with sham control, CLP induced elevation of IL-6 in aged mice. Administration of rhMG53 (5 mg/kg, intraperitoneal injection) or hCitH3-mAb (20 mg/kg, tail vein injection) mitigated the elevation of IL-6. Co-administration of rhMG53 and hCitH3-mAb nearly completely abolished the elevation of IL-6.

### Example 17: hCitH3-mAb can be Used to Treat Virus-Induced Multi-Organ Injuries

### Methods

Virus stocks—Influenza virus strains A/Puerto Rico/8/34 (H1N1) (PR8, provided by Dr. Jacob Yount at Ohio State University) were propagated in 10-day embryonated chicken eggs (Charles River Laboratories, Wilmington MA) and tittered as previously described.

Mouse Studies—Aging male C57BL/6J mice (22-24 months old) were obtained from National Institute of Aging. Mice were infected intranasally under anesthesia with isoflurane according to protocols approved by the Institutional Animal Care and Use Committee at University of Virginia. Mouse infections were performed with a dose of 150 TCID50 of PR8. Two doses of humanized CitH3 antibody (hCitH3-mAb) were administrated via tail vein injection with a dose of 20 mg/kg at 24 and 72 hours after flu virus infection. rhMG53 protein was administered via tail vein injection daily for 7 days with a dose of 2 mg/kg. The first dose of rhMG53 was given at 24 hours after flu infection. Mouse body weight was monitored and recorded on a daily basis. Greater than 20% weight loss was the primary criteria used for humane endpoint euthanasia in mouse experiments. For determining lung cytokine levels, tissues were collected and homogenized in 1 ml of PBS, flash-frozen, and stored at −80° C. prior to analysis via ELISA. Mouse IFNβ, IL-6 ELISAs were performed on supernatant from lung homogenates supernatants using the respective R&D Systems® Duoset® ELISA kits (Minneapolis, MN, catalogue #DY8234-05, and DY406, respectively) according to manufacturer's instructions. Lung tissue samples were fixed in 10% neutral-buffered formalin at 4° C. for 24 hours and then transferred to 70% ethanol. Lungs were embedded in paraffin, sectioned, stained with H&E, and imaged via the Leica DMi8 Widefield microscopy. Plasma samples were collected at the time of animal euthanasia and subjected to the multiplex assay for mouse cytokines (GM-CSF, IFNγ, IL13, LIX) at the Core facility for flow cytometry at University of Virginia Medical Center.

Western blots—Snap-frozen lung tissues were processed in radio immunoprecipitation assay (RIPA) lysis buffer (10 mM Tris-HCl, pH 7.2, 150 mM NaCl, 1% NP-40, 0.5% SDS, and 0.5% deoxycolate), supplemented with a cocktail of protease inhibitors (Sigma-Aldrich®) and phosphatase inhibitors (Thermo Fisher Scientific) for Western blot analyses. Mouse lung lysates were resolved by 10% SDS-PAGE and transferred onto polyvinylidene fluoride (PVDF) membranes. The blots were washed with Tris-buffered saline with Tween 20 (TBST), blocked with 5% milk in TBST for 1 hour, and incubated with different antibodies as indicated in the figures. Immunoblots were visualized with an Pierce™ ECL Plus kit (Pierce).

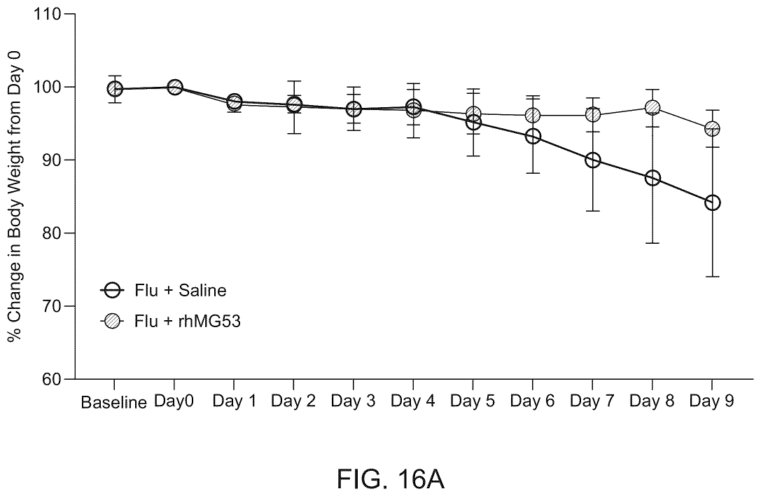

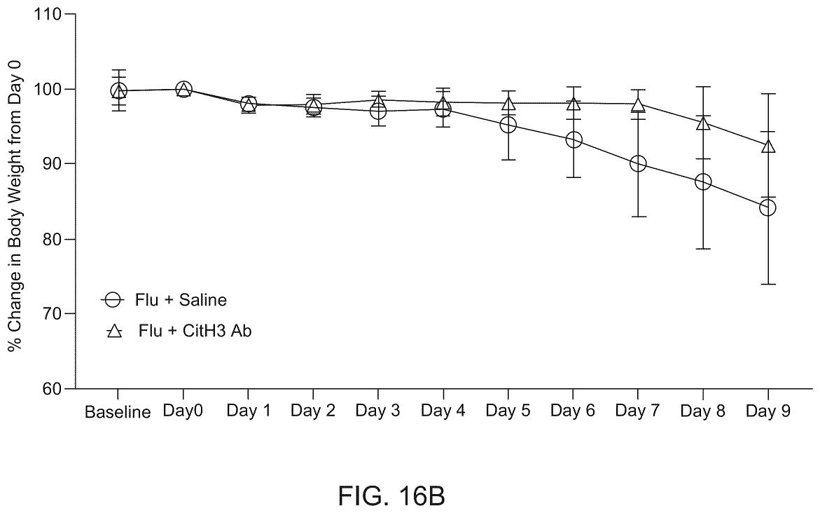

Changes of body weight (% of initial body weight) were plotted for animals infected with flu virus and treated with rhMG53 (FIG. 16A) or hCitH3-mAb (FIG. 16B) versus animals treated with saline as control group.

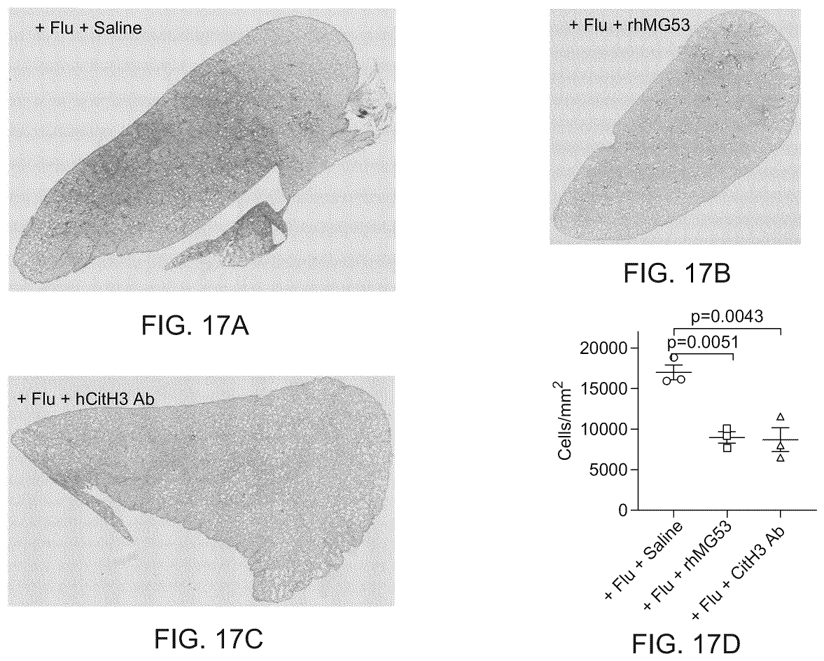

Representative microscope images for lung tissue section with H&E staining for mouse infected with flu virus and treated with saline, rhMG53, or hCitH3-mAb are shown in FIG. 17A-17D. The quantitative analysis of cellulalization in the tissue section were conducted using the ImageJ software. Decreased cell infiltration was observed in the lung tissue collected from animals at 9 days after flu virus infection and treated with rhMG53 or hCitH3-mAb compared to animals treated with the saline group. (n=3 per group, p values calculated using one-way ANOVA with Tukey's multiple-comparison test).

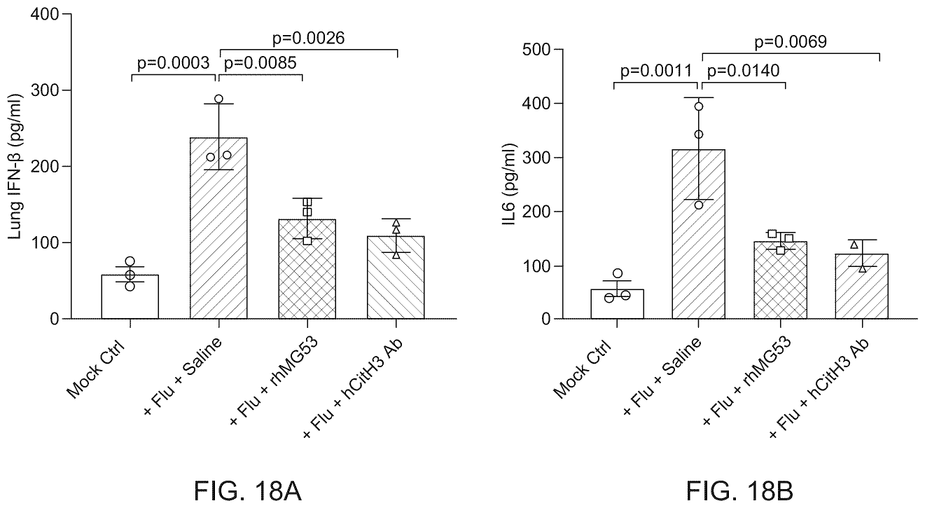

FIGS. 18A and 18B show results of ELISA quantification of IFNβ and IL-6 in the lung tissue collected from mice without flu virus infection, or mice with 9-day flu virus infection and treated with saline, rhMG53 or hhCitH3-mAb (n=3 per group, p values calculated using one-way ANOVA with Tukey's multiple-comparison test). The results demonstrated that, similar to rhMG53, the administration of hCitH3-mAb significantly reduced cytokine storms in the lung tissue following virus infection.

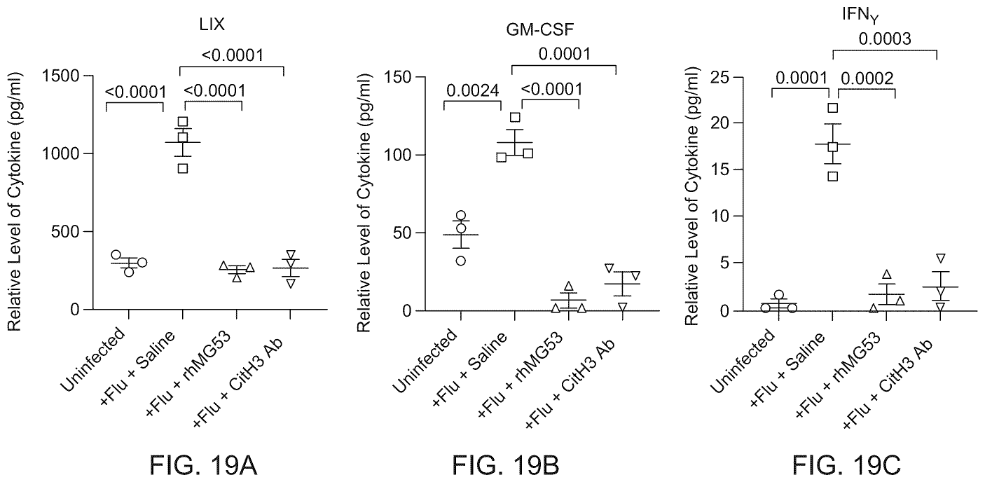

Multiplex assay for mouse cytokines showed increased plasma level of inflammatory markers, including GM-CSF, IFNγ, and LIX, in mice with flu virus infection compared to uninfected control animals, which were significantly decreased in animals treated with rhMG53 or hCitH3-mAb (n=3 per group, p values calculated using one-way ANOVA with Tukey's multiple-comparison test) (FIG. 19A-19C).

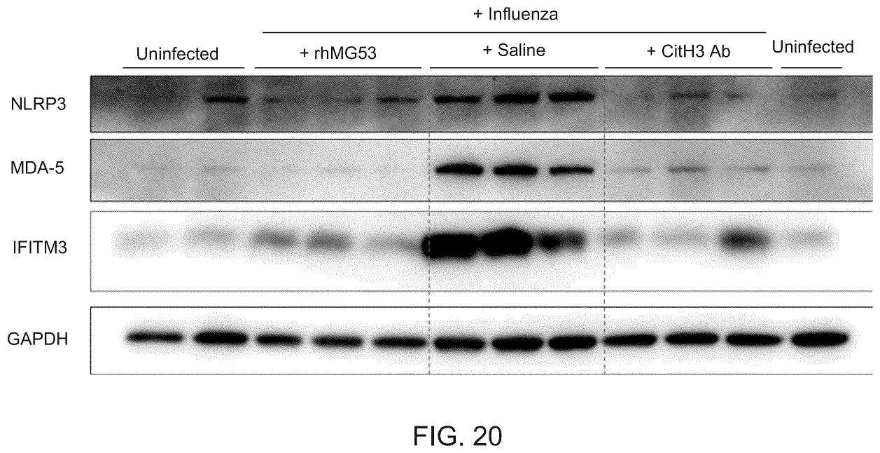

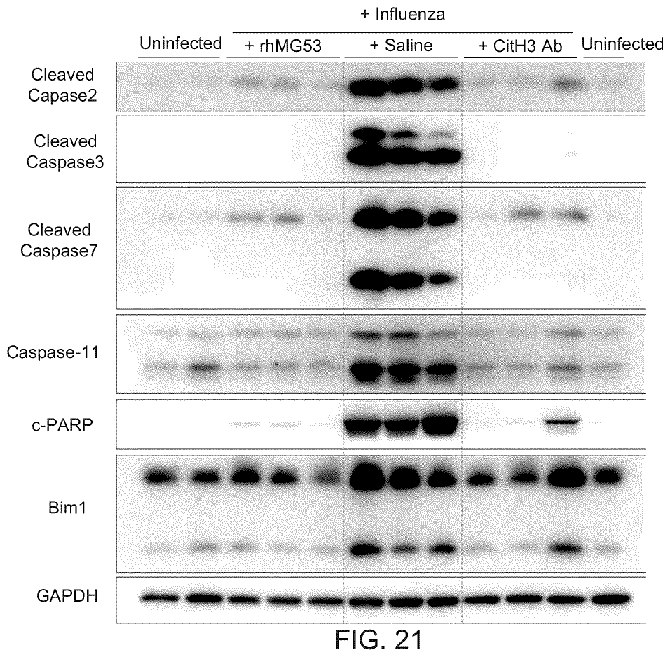

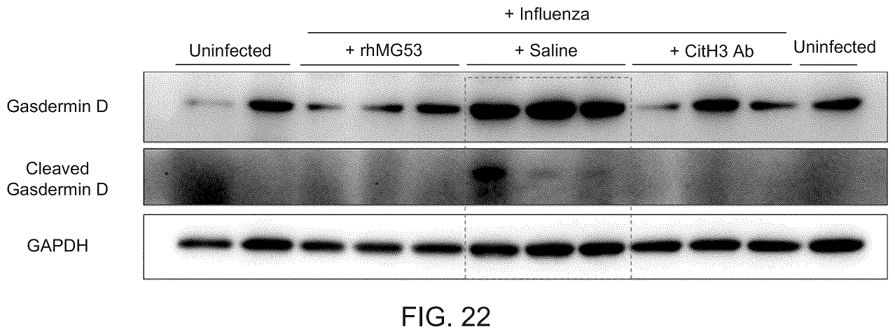

Western blot analysis showed that treatment with rhMG53 or hCitH3-mAb suppressed the inflammation as indicated by the decreased level of NLRP3, MDA-5, IFITM3 following 9-day influenza virus (FIG. 20). Furthermore, treatment with rhMG53 or hCitH3-mAb suppressed the apoptosis as indicated by the decreased level of cleaved caspase 2/3/11 and PARP (Poly (ADP-ribose) polymerase) following 9-day influenza virus infection (FIG. 21). Suppression of expression of full-length GasderminD and decreased level of cleaved Gasdermin D was also noted (FIG. 22). Since cleavage of Gasdermin D is a known trigger for pyropoptic tissue injury, mitigation of Gasdermin D cleavage by hCitH3-mAb supports the beneficial effects of hCitH3-mAb in protecting against virus-induced lung injury.

### Example 18: hCitH3-mAb can be Used to Treat Sterile-Inflammation Induced Multi-Organ Injuries

### Methods

Animals—Male wild-type mice (C57BL/6, 10-12 weeks age) were purchased form Jackson Laboratory. Animal handling and surgical procedures were performed according to protocols approved by the Institutional Animal Care and Use Communities (IACUC) of the University of Virginia in compliance with the National Institutes of Health Guidelines for Care and Use of Experimental Animals.

Liver Ischemia—12 weeks of male mice were used for the partial hepatic warm ischemia surgery. Before surgery, all surgical tools were sterilized by autoclaving. Mice were anesthetized in the induction chamber containing 5% isoflurane in 100% oxygen at a 1 liter/min flow rate. Then, mice were placed on a nose cone connected with a continuous oxygen with 5% isoflurane in the supine position on the warm pad. Body temperature was monitored by rectal temperature probe and was maintained at 37° C. by a warming pad. A partial hepatic (70%) warm ischemia was used. The ischemia was initiated by abdominal hair removal and cleaned with betadine three times. Under the dissection microscope, the liver hilum was dissected free of surrounding tissue. All structure in the portal triad (hepatic artery, portal vein, bile duct) to the left and median liver lobes were occluded with a microvascular clamp for 90 min, then reperfusion was initiated by removal of the clamp. During the ischemia, it was confirmed by visualizing the pale blanching of the ischemic lobes. Also, the evidence for reperfusion was based on immediate color change of ischemic lobes. Then, the abdominal incision was closed using 5-0 polypropylene suture. Animals were sacrificed at time points of 0.5, 6, 24, 72 hrs. after reperfusion for serum, liver, heart, lung, kidney, and spleen collection.

Antibody treatment—One group of mice received hCitH3-mAb, and another group of mice received human IgG antibody (Sigma-Aldrich®, I4506) at 20 mg of antibody per kg of mouse by IV injection immediately after reperfusion and at day 3 after surgery.

Echocardiography—For echocardiography, mice underwent baseline echocardiography followed by liver ischemia reperfusion surgery. Then, all surviving mice were subjected to follow-up echocardiography at day 7, day 14 after surgery.

Western blot analysis—Total protein was extracted from snap-frozen liver tissues using a protein lysis buffer containing protease inhibitors. The denatured proteins were resolved onto 4 to 12% gradient gel (Invitrogen™, NP0336BOX) and the separated proteins were transferred to PVDF membranes. Membranes are stained with Ponceau S solution to show equal protein loading for each sample. Membranes were further probed with primary antibody and incubate at 4° C. overnight. Then, they were washed with TBST and probed with secondary antibody. Protein expression is detected by Invitrogen Imaging system using chemiluminescent substrate (Thermo Scientific™, PI34580). Antibodies used were as follows: MPO (R&D Systems®, AF3667), GAPDH (Cell Signaling Technology®, 2118S). For the serum samples, 1 μl of serum was loaded into the SDS-PAGE for western blot analysis.

In addition to the in vivo studies with hCitH3-mAb using the mouse model of sepsis and virus-induced multi-organ failures, a surprising and exciting finding is that hCitH3-mAb is effective in treating sterile-inflammation induced tissue injuries.

Experiments using ischemia-reperfusion induced liver injury were conducted, and results show that hCitH3-mAb has significant benefits for treating the sterile-inflammation induced liver injuries. More surprisingly, ischemia-reperfusion induced liver injury can propagate to myocardial infarction as reflected by the reduced ejection fraction (EF) of the mouse heart. Interestingly, the administration of hCitH3-mAb can mitigate the decline of EF in mice subjected to ischemia-reperfusion induced liver injury.

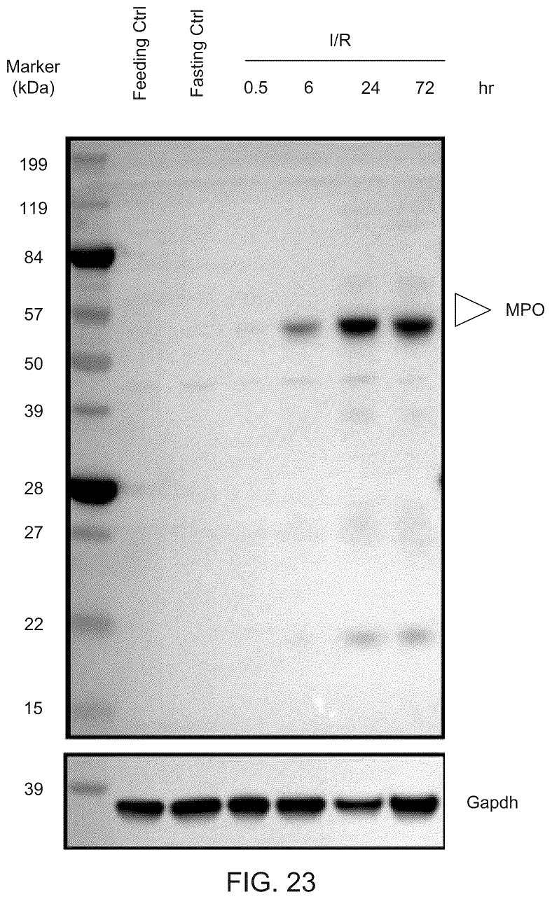

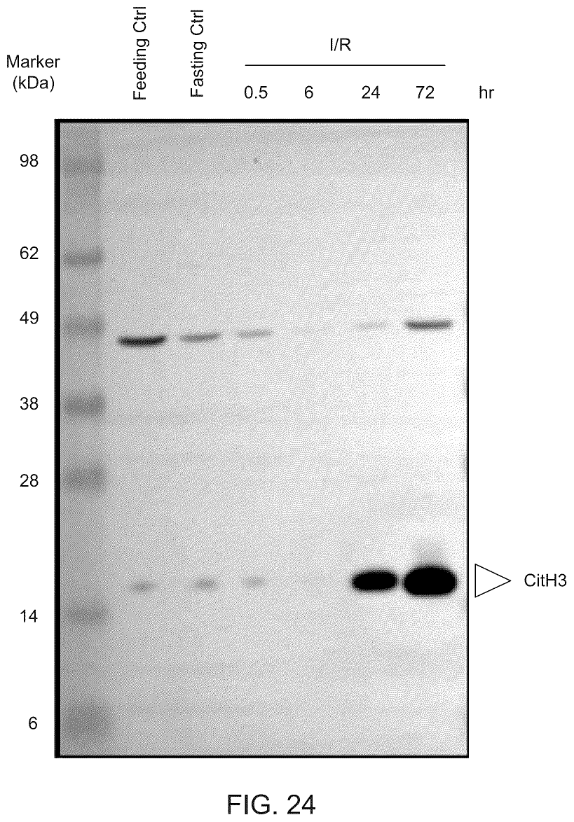

Western blot analysis showed that the expression of MPO, a neutrophil marker, was observed in ischemia-reperfusion induced liver injury with upregulation detected from 6 hours up to 72 hours after reperfusion (FIG. 23), and that the expression of CitH3 was increased in ischemia-reperfusion induced liver injury at 24 hours after reperfusion and continued to increase up to 72 hrs (FIG. 24).

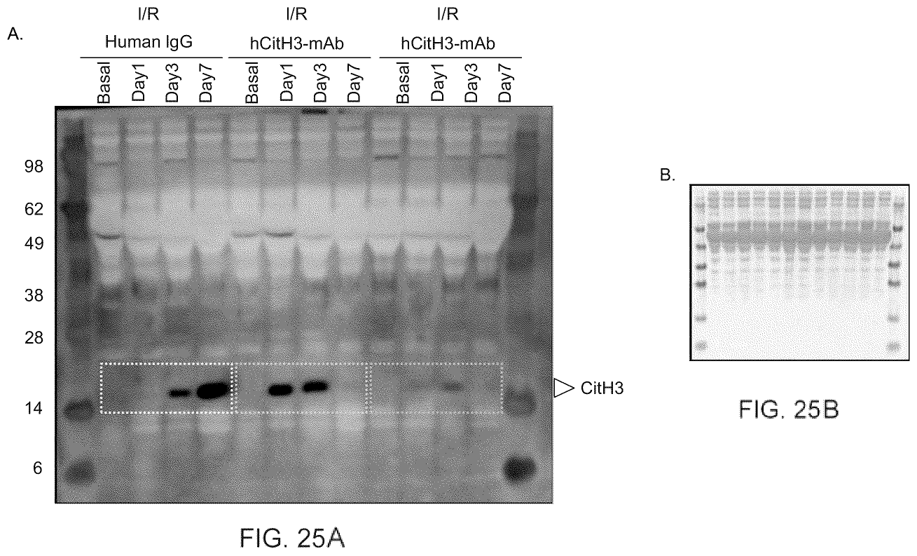

Results showed that hCitH3-mAb treatment eliminated CitH3 level in serum (FIG. 25A). Mice that underwent liver ischemia and reperfusion were injected with human IgG antibody or hCitH3-mAb via i.v. immediately after reperfusion. CitH3 levels were determined in serum samples collected before surgery and on days 1, 3, and 7 after surgery. (FIG. 25B) shows the Ponceau S staining for the loading control.

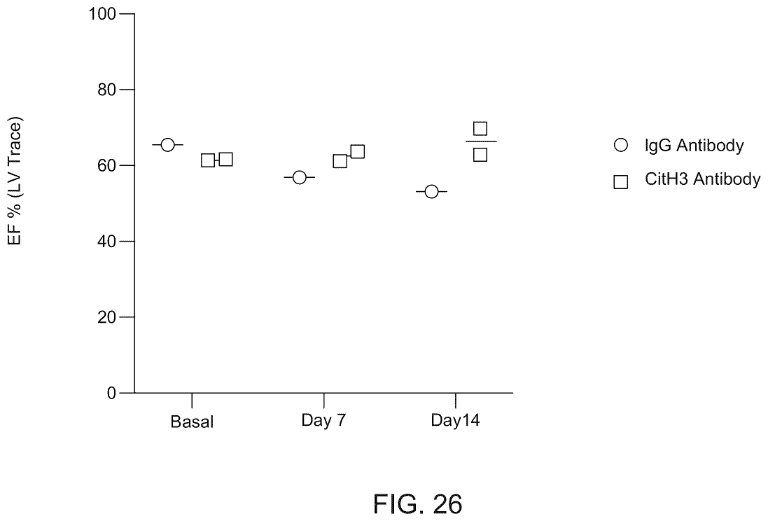

FIG. 26 shows neutralization of CitH3 with hCitH3-mAb restores cardiac function following liver I/R, whereas the group treated with human IgG antibody exhibit impairment of cardiac function.

These findings support a broader value of hCitH3-mAb to treat multi-organ injuries under conditions of sterile-inflammation.

### Example 19: hCitH3-mAb Protects Against Ischemic-Stroke in Mice

### Methods

C57BL/6J mice (Male, 16-20 weeks old, body weight 25-30 g) were obtained from Jackson Lab. Mice were established under anesthesia with isoflurane according to protocols approved by the IACUC at University of Virginia. A commercial silicon coated monofilament (Doccol Corporation, Sharon, MA) was introduced into the internal carotid artery (ICA) via a cut in the external carotid artery (ECA) until the monofilament occludes the base of the MCA. Brain ischemia reperfusion was established using transient middle cerebral artery occlusion (tMCAO) for 60 min according to our previous studies.

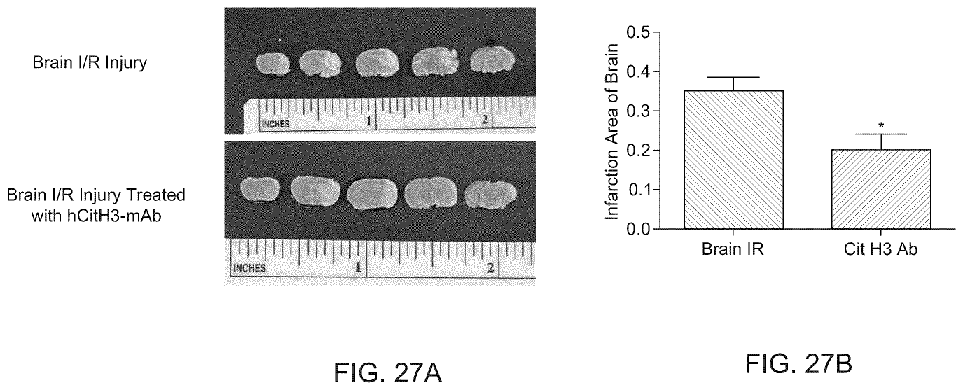

Mice were divided into two groups: (i) tMCAO group treated with IgG as control, (ii) tMCAO group treated with hCitH3-mAb. IgG and hCitH3-mAb were administrated via tail vein injection with a dose of 20 mg/kg immediately after reperfusion. At 24 h after reperfusion, the whole-brain tissue was harvested and frozen at −20° C. for 10 min at the time of animal euthanasia, and then sliced into five serial 2 mm coronal sections and incubated in a 1% 2,3,5-triphenyltetrazolium chloride (TTC; Sigma Aldrich®) for 20 minutes at 37° C. and turned over the slice gently every 5 minutes, fixed in 4% paraformaldehyde in phosphate buffer at 4° C. The white tissue indicated the infarct part, whereas the red meant normal. The extent of ischemic infarction was traced, and the integrated volume was calculated using Image J software. Results are shown in FIG. 27A-27B.

### Example 20: hCitH3-mAb can be Used as Therapeutic to Treat Diabetes and Diabetic Wounds

### Methods

14 week-old B6.BKS(D)-Lepr^{db}/J (db/db) mice were purchased from the Jackson Laboratory (Bar Harbor, ME). After anesthesia using 1.5% isoflurane, the dorsal hair of mice was removed using a depilatory cream (Nair), and a 5 mm round full-thickness skin excision wound was made at midline of the back 2-cm caudal ward from the skull of each mouse. hCitH3-mAb (20 mg/kg) and human IgG (Sigma-Aldrich, I4506; 20 mg/kg) was subcutaneously injected from post-injury day 0 and day 3. The wounds were covered with a hydrocolloid dressing (Tegaderm™; 3M Health Care, St. Paul, MN) to maintain a moist environment, and changed daily. Wound area was measured at post-injury day 2, 4, 7, 10, and 12. The data revealed benefits of hCitH3-mAb to improve healing of dermal wounds in the db/db mice (n=3 each).

Wound healing is impaired in diabetes, with diabetic foot ulcer (DFU) presenting significant risks of morbidity and mortality. The combination of neuropathy and vasculopathy contributes to DFUs, but the underlying cellular and molecular mechanisms impairing tissue healing in diabetes remain poorly understood. This lack of understanding limits therapeutic strategies beyond glucose control, revascularization, and traditional wound care.

NETosis is activated by proinflammatory cytokines and reactive oxygen species, particularly in diabetes, known for chronic inflammation and oxidative stress. Citrullination of histone H3 (CitH3), catalyzed by peptidylarginine deiminases (PAD2 and PAD4), plays a critical role in initiating NETs-induced immune cell death and tissue injury. Elevated CitH3 has been identified as a risk factor for wound healing impairment and amputation in patients with DFU.

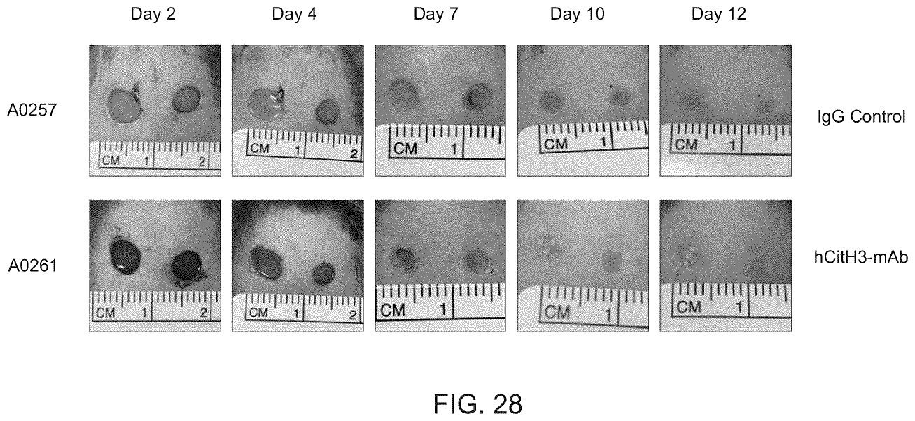

FIG. 28 shows that hCitH3-mAb enhances wound healing in diabetic mice, and the combination of hCitH3-mAb and rhMG53 adds synergy to treat diabetic ulcer.

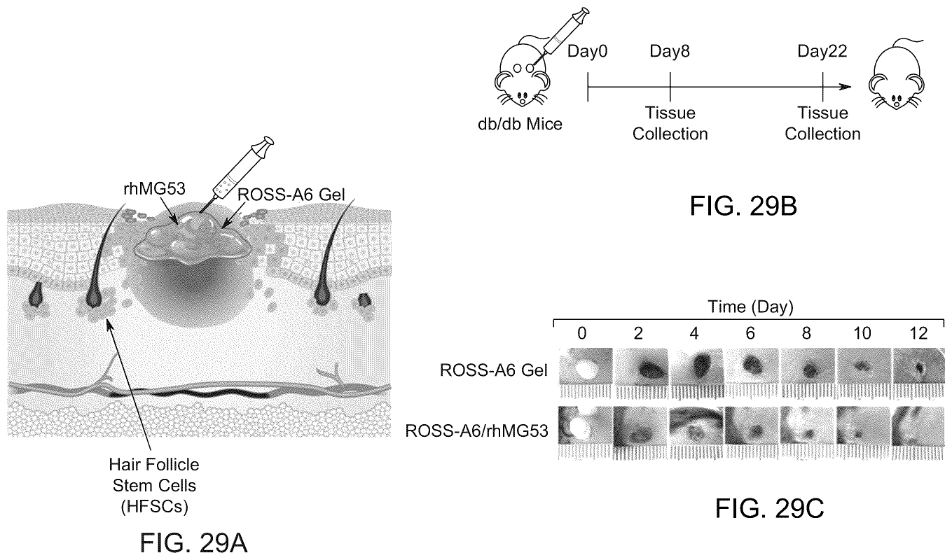

Previously we demonstrated that hydrogel formulation of recombinant human MG53 (rhMG53) protein is effective in treating dermal wounds in db/db mice. We show that co-treatment of hCitH3-mAb and rhMG53 can have synergy to control inflammation (via hCitH3-mAb) and improve healing (via rhMG53) and therefore to effectively treat diabetic ulcer (FIG. 29A-29C). Composition of the ROSS-A6 hydrogel for encapsulation of rhMG53 protein for wound healing application was previously described. It was previously shown that sustained delivery of rhMG53 promotes diabetic wound healing and hair follicle development. Surprisingly, our results indicate that the combination of hCitH3-mAb and rhMG53 can provide synergy to enhance treatment of diabetic ulcer, possibly by complementing functions: rhMG53 in promoting wound healing and hCitH3-mAb in mitigating NETosis dysfunction associated with diabetes.

### Other Embodiments

It is to be understood that while the invention has been described in conjunction with the detailed description thereof, the foregoing description is intended to illustrate and not limit the scope of the invention, which is defined by the scope of the appended claims. Other aspects, advantages, and modifications are within the scope of the following claims.

What is claimed is:

1. A humanized antibody or antigen-binding fragment thereof that binds to CitH3 (citrullinated histone H3) comprising:
a heavy chain variable region (VH) variant comprising any of SEQ ID NO: 7, SEQ ID NO: 8, SEQ ID NO:9, or SEQ ID NO: 10, the VH region comprising complementarity determining regions (CDRs) 1, 2, and 3, wherein the VH CDR1 region comprises an amino acid sequence that is at least 80% identical to a selected VH CDR1 amino acid sequence, the VH CDR2 region comprises an amino acid sequence that is at least 80% identical to a selected VH CDR2 amino acid sequence, and the VH CDR3 region comprises an amino acid sequence that is at least 80% identical to a selected VH CDR3 amino acid sequence; and
a light chain variable region (VL) variant comprising any of SEQ ID NO: 11, SEQ ID NO:12, or SEQ ID NO:13, the VL region comprising CDRs 1, 2, and 3, wherein the VL CDR1 region comprises an amino acid sequence that is at least 80% identical to a selected VL CDR1 amino acid sequence, the VL CDR2 region comprises an amino acid sequence that is at least 80% identical to a selected VL CDR2 amino acid sequence, and the VL CDR3 region comprises an amino acid sequence that is at least 80% identical to a selected VL CDR3 amino acid sequence,
wherein the selected VH CDRs 1, 2, and 3 amino acid sequences and the selected VL CDRs, 1, 2, and 3 amino acid sequences are one of the following:
(1) the selected VH CDRs 1, 2, 3 amino acid sequences are set forth in SEQ ID NOs: 1, 2, 3, respectively, and the selected VL CDRs 1, 2, 3 amino acid sequences are set forth in SEQ ID NOs: 4, 5, 6, respectively; and
(2) the selected VH CDRs 1, 2, 3 amino acid sequences are set forth in SEQ ID NOs: 16, 17, 18, respectively, and the selected VL CDRs 1, 2, 3 amino acid sequences are set forth in SEQ ID NOs: 19, 20, 21, respectively.

2. A humanized antibody or antigen-binding fragment thereof that binds to CitH3 comprising
a heavy chain variable region (VH) comprising an amino acid sequence that is at least 90%, 95%, or 100% identical to SEQ ID NO: 7, 8, 9, 10, or 14, and a light chain variable region (VL) comprising an amino acid sequence that is at least 90%, 95%, or 100% identical to SEQ ID NO: 11, 12, 13, or 15.

3. The antibody or antigen-binding fragment thereof of claim 1, wherein the VH region comprises SEQ ID NO: 9 and the VL region comprises SEQ ID NO: 13.

4. The antibody or antigen-binding fragment thereof of claim 3, wherein the antibody or antigen-binding fragment is a single-chain variable fragment (scFv) or a multi-specific antibody.

5. The antibody or antigen-binding fragment thereof of claim 2, wherein the antibody or antigen-binding fragment is a single-chain variable fragment (scFv) or a multi-specific antibody.

6. A nucleic acid comprising a polynucleotide encoding a humanized antibody or antigen-binding fragment thereof according to claim 1, comprising:
(1) an immunoglobulin heavy chain or a fragment thereof comprising a heavy chain variable region (VH) comprising complementarity determining regions (CDRs) 1, 2, and 3 comprising the amino acid sequences set forth in SEQ ID NOs: 1, 2, and 3, respectively, and wherein the VH, when paired with a light chain variable region (VL) comprising the amino acid sequence set forth in SEQ ID NO: 11, 12, 13, or 15, binds to CitH3; or
(2) an immunoglobulin light chain or a fragment thereof comprising a VL comprising CDRs 1, 2, and 3 comprising the amino acid sequences set forth in SEQ ID NOs: 4, 5, and 6, respectively, and wherein the VL, when paired with a VH comprising the amino acid sequence set forth in SEQ ID NO: 7, 8, 9, 10, or 14, binds to CitH3.

7. A vector comprising one or more of the nucleic acids of claim 5.

8. A cell comprising one or more vector of claim 7.

9. A method of producing an antibody or an antigen-binding fragment thereof, the method comprising
(a) culturing the cell of claim 8 under conditions sufficient for the cell to produce the antibody or the antigen-binding fragment; and
(b) collecting the antibody or the antigen-binding fragment produced by the cell.

10. A method of treating a subject having a disease, the method comprising administering a therapeutically effective amount of a composition comprising the antibody or antigen-binding fragment thereof of claim 1 to the subject, wherein the disease is cancer, an immune disorder, an infectious disease, sepsis, a chronic wound, or ischemia.

11. The method of claim 10, wherein the disease is associated with NETosis.

12. The method of claim 10, wherein the disease is cancer and the subject has a solid tumor or hematological malignancy.

13. The method of claim 12, wherein the cancer is associated with CitH3-induced NETosis.

14. The method of claim 10, wherein the infectious disease is caused by bacteria or a virus.

15. The method of claim 10, wherein the chronic wound is a dermal wound.

16. The method of claim 15, wherein the dermal wound is a diabetic foot ulcer.

17. The method of claim 10, wherein the ischemia is liver ischemia, brain ischemia, or cardiac ischemia.

18. The method of claim 10, further comprising co-administration of another therapeutic compound, wherein the compound is rhMG53.

19. A pharmaceutical composition comprising the humanized antibody or antigen-binding fragment thereof of claim 1, and a pharmaceutically acceptable carrier.

20. A pharmaceutical composition according to claim 19, further comprising another therapeutic compound, wherein the compound is rhMG53.


SEQUENCE LISTING (22 sequences)

SEQ ID NO: 1 - protein, 5 aa, synthetic construct
DYYMH

SEQ ID NO: 2 - protein, 17 aa, synthetic construct
WIDPENGNTIYDPKFQG

SEQ ID NO: 3 - protein, 9 aa, synthetic construct
FGNYVWFAY

SEQ ID NO: 4 - protein, 16 aa, synthetic construct
RSSQSIVHSNGITYLE

SEQ ID NO: 5 - protein, 7 aa, synthetic construct
KVSNRFS

SEQ ID NO: 6 - protein, 9 aa, synthetic construct
FQGSHVPFT

SEQ ID NO: 7 - protein, 118 aa, synthetic construct
EVQLVQSGAEVKKPGATVKISCKASGFNIKDYYMHWMQQRPGKGLEWIGWIDPENGNTIYDPKFQGRVTITSDTSTDTAYLELSSLRSEDTAVYYCAPFGNYVWFAYWGQGTLVTVSS

SEQ ID NO: 8 - protein, 118 aa, synthetic construct
EVQVVQSGAEVKKPGATVKISCKASGFNIKDYYMHWMKQRPEQGLEWIGWIDPENGNTIYDPKFQGRVTITSDTSTDTAYLELSSLRSEDTAVYYCAPFGNYVWFAYWGQGTLVTVSS

SEQ ID NO: 9 - protein, 118 aa, synthetic construct
EVQVVQSGAEVKKPGATVKISCKASGFNIKDYYMHWMKQRPEQGLEWIGWIDPENGNTIYDPKFQGRATITSDTSTDTAYLELSSLRSEDTAVYYCAPFGNYVWFAYWGQGTLVTVSS

SEQ ID NO: 10 - protein, 118 aa, synthetic construct
EVQVQQSGAEVKKPGATVKLSCKASGFNIKDYYMHWMKQRPEQGLEWIGWIDPENGNTIYDPKFQGRATITSDTSTDTAYLELSSLRSEDTAVYYCAPFGNYVWFAYWGQGTLVTVSS

SEQ ID NO: 11 - protein, 113 aa, synthetic construct
DVVMTQSPLSLPVTLGQPASISCRSSQSIVHSNGITYLEWFQQRPGQSPRLLIYKVSNRFSGVPDRFSGSGSGTDFTLKISRVEAEDVGVYYCFQGSHVPFTFGQGTKLEIKR

SEQ ID NO: 12 - protein, 113 aa, synthetic construct
DVVMTQSPLSLPVTLGQPASISCRSSQSIVHSNGITYLEWYLQRPGQSPKLLIYKVSNRFSGVPDRFSGSGSGTDFTLKISRVEAEDVGLYYCFQGSHVPFTFGQGTKLEIKR

SEQ ID NO: 13 - protein, 113 aa, synthetic construct
DVLMTQSPLSLPVTLGQPASISCRSSQSIVHSNGITYLEWYLQRPGQSPKLLIYKVSNRFSGVPDRFSGSGSGTDFTLKISRVEAEDVGLYYCFQGSHVPFTFGQGTKLEIKR

SEQ ID NO: 14 - protein, 118 aa, synthetic construct
EVQVQQSGAELVRPGALVKLSCKASGFNIKDYYMHWMKQRPEQGLEWIGWIDPENGNTIYDPKFQGKATITSDTSSNTAYLQLNSLTSEDTAVYYCAPFGNYVWFAYWGQGTLVTVSA

SEQ ID NO: 15 - protein, 113 aa, synthetic construct
DVLMTQTPLSLPVSLGDQASISCRSSQSIVHSNGITYLEWYLQKPGQSPKLLIYKVSNRFSGVPDRFSGSGSGTDFILKISRVEAEDLGLYYCFQGSHVPFTFGSGTKLEIKR

SEQ ID NO: 16 - protein, 7 aa, synthetic construct
GFNIKDY

SEQ ID NO: 17 - protein, 6 aa, synthetic construct
DPENGN

SEQ ID NO: 18 - protein, 9 aa, synthetic construct
FGNYVWFAY

SEQ ID NO: 19 - protein, 16 aa, synthetic construct
RSSQSIVHSNGITYLE

SEQ ID NO: 20 - protein, 7 aa, synthetic construct
KVSNRFS

SEQ ID NO: 21 - protein, 9 aa, synthetic construct
FQGSHVPFT

SEQ ID NO: 22 - protein, 30 aa, synthetic construct
ACTKQTACKSTGGKAPCKQLATKAACKSAP